SEQUENCE LISTING (2 sequences)

SEQ ID NO: 1 - protein, 721 aa, Ralstonia pickettii
MTTEAKCPFSGHAPAASHAFGGGTANKDWWPNQLRVDLLNQHSEKSDPLGSNFNYRKSFNAIDYDALKADLRRLMTDSQDWWPADFGHYGPQFIRMAWHAAGTYRTGDGRGGAGRGQQRFAPLNSWPDNVNIDKSRRLLWPIKQKYGQAISWADLLILTGNVALETMGFRTFGFAAGREDTWEPDNDVYWGNETKWLEATRYSGERNLANPLAAVQMGLIYVNPEGPEHAHGDPLAAAKDIRETFARMAMDDEETVALIAGGHTFGKTHGAGPASHVGADVEAAPLEAQGLGWASTFGTGKGADAITSGLEVTWTQTPAQWSNFFFENLFKYEWVQEKSPAGALQWVAKDAEAIIPGPTPDSPKRRPTMLTTDLSLRFDPAYEKISRRFLDNPQAFAEAFARAWFKLTHRDLGPKSRYLGPEVPREDLIWQDPLPTATHKPTEADIADLKAKIAASGLSASELVAVAWASASTFRGSDKRGGANGARIRLAPQKDWAVNQPVAGTLARLEEIQRASGKASLADVIVLAGSVGIELAAKAAGTTITVPFTPGRVDATAEQTDATSFSVLEPVADGFRNYQKTQFAVPGEVLLLDRAQLLTLTAPELTVLIGGLRAININADGSQHGVFTSTPGALTNDFFVNLLDMNTEWKPAGDIYEGYDRKTGERKWTGTRVDLVFGSNSILRALAEVFGSADGKERFISEFVAAWVKVMNLDRFDLAAA

SEQ ID NO: 2 - protein, 431 aa, Ralstonia pickettii
MNSLIAALLPIGVFGSCPFMDIGTPPSLKAEVISDSDFKTALEGLDIEALEQDLRILMTDSQTCWPADDGHYGGFMIRLAWHCAGTFRTSDQKGGCGGAGIRFPPESDWEDNGNLDKARALLVPIKQKYGDALSWGDLISFAGTVAIRDMGGPTNPHCFGRVDDADGNKSDIFGVTDSWQDTDCVVQGNCQEPMGAVKVGLIYVNPEGPLNDPNDLNSGQNPDPEKSAVEIREVFGRMGMNDSETASLIAGGHAFGKCHGAGVMTSGFEGPWTTTPSQWTNQFLTGMLDEEWEQVATPSGSAVQWQTKDRTSILAGTMRLTADLALVNDDAYLALAKHWVCDQQKLDIAFAASWKKLVESGGGWLPVEDRRCEPESKATGQQTDPQKNFHSVCATTTTDDSQEASTATKFCLSFLAVVLTPFSLGVHSWLA

(19) United States
(12) Patent Application Publication
(10) Pub. No.: US 2023/0167469 A1
(43) Pub. Date: Jun. 1, 2023

(54) ENZYMATIC DEGRADATION OF PLASTIC POLYALKENE POLYMERS BY KATG ENZYME

(71) Applicant: MELLIZYME BIOTECHNOLOGY LIMITED, London (GB)

(72) Inventors: Jacob Leon Nathan, London (GB); Douglas Bruce Kell, London (GB)

(21) Appl. No.: 17/917,179

(22) PCT Filed: Apr. 6, 2021
(86) PCT No.: PCT/GB2021/050844

§ 371 Date: Oct. 5, 2022

(30) Foreign Application Priority Data
Apr. 6, 2020 (GB) 2005073.8

(51) Int. Cl. C12P 7/44; C12N 9/08

(52) U.S. Cl. CPC C12P 7/44; C12N 9/0065; C12Y 111/01021

(57) ABSTRACT

The present invention relates to KatG enzymes and enzyme compositions and their uses in enzymatic degradation of plastics.

### FIELD OF THE INVENTION

This invention relates to methods for the enzymatic degradation of plastics, including in particular synthetic plastic polyalkene (PA) polymers, such as polyethylene (PE), polypropylene (PP), polyvinyl chloride (PVC), polystyrene (PS) and chlorinated polyethylene (CPE) polymers; to novel enzyme and enzyme compositions and their uses in enzymatic degradation of plastics.

### BACKGROUND TO THE INVENTION

The global production of synthetic plastic polymers from fossil fuel feedstocks (coal, natural gas and oil) has continuously increased over the last 50 years, and reached 322 million tons in 2015. Over 30 million tons of post-consumer plastic waste is generated annually in Europe alone, and only a small fraction of this waste is recovered by recycling and energy generation processes.

In consequence, plastic pollution is now recognized as a global ecological disaster with huge social and economic impact. The need for improvements in the management of plastic waste is therefore acute.

However, plastic waste is recalcitrant, and persists in the environment for very long periods of time (hundreds to thousands of years). There is therefore considerable interest in enzymatic processes for its degradation, which could be developed into processes for environmental remediation, as well as industrial reactors for the recycling or degradation of waste plastics.

Degradation of Plastics with Heteroatomic Backbones (PET and PUR)

Various enzymes have been identified and developed for the degradation of synthetic plastics containing a heteroatomic backbone and ester bonds (such as polyurethanes (PUR) and polyethylene terephthalate (PET)), the general backbone structures of which are shown below:

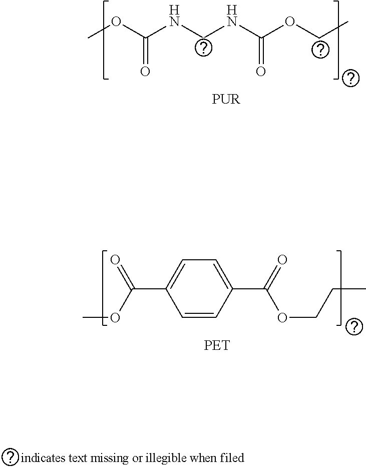

This area is reviewed in Wei and Zimmermann (2017) Microbial Biotechnology 10: 1308-1322 and, more recently, by Danso et al. (2019) Applied and Environmental Microbiology 85: e01095-19.

Polyester waste plastics are now widely recognized as particularly tractable, since their ester bonds can be targeted by a variety of different enzymes. These polyester waste plastics therefore represent the “low hanging fruit” for industrial enzymatic waste plastic degradation.

Enzymes useful in this context include various proteases, lipases, carboxylesterases and esterases (including in particular cutinases). Engineered variant forms having increased activity and/or thermostability have also been described. For example, various natural and engineered esterases are described in WO2020021118, WO2020021117, WO2020021116, WO2018011284, WO2018011281, WO2016146540 and WO2016062695, the contents of which are hereby incorporated by reference.

Various natural and engineered proteases are described in WO2019122308 and WO2018109183, the contents of which are also hereby incorporated by reference. The high-resolution X-ray crystal structure of the I. sakaiensis PETase and engineered variants are described in Austin et al. (2018) PNAS 115(19): E4350-E4357.

Polyester plastics which can be degraded in this way include PET, but also other polyesters such as polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polybutylene succinate terephthalate (PBST), polyethylene succinate (PES), poly(butylene succinate/terephthalate/isophthalate)-co-(lactate) (PBSTIL) and polyethylene naphthalate (PEN)).

Industrial processes for the enzymatic biodegradation of such polyester polymers are described in WO2014079844, WO2015067619, WO2015097104 and WO2015173265.

### Polyalkene (PA) Enzymatic Degradation

The developments discussed above in relation to polyester polymers have the potential to improve the disposal and recycling of plastic waste. However, synthetic plastic polyalkene (PA) polymers having a C—C backbone make up most of the plastic discarded as waste, and this class of polymers are uniquely resistant to biodegradation. These PA polymers include various types of polyethylene (PE), and also structurally-related polyalkene polymers with a C—C backbone (which may be considered as substituted polyethylenes), including polypropylene (PP), polystyrenes (PS), polyvinyl chloride (PVC) and chlorinated polyethylene (CPE). Of these plastics, PE and PP represented over 90% of total plastic production in 2015 (The Economist, “Single-use refuse”, Oct. 2 2015 edition).

The biodegradation of these PA polymers is hampered by the lack of hydrolysable functional groups in the C—C backbone, as is apparent from the structures shown below:

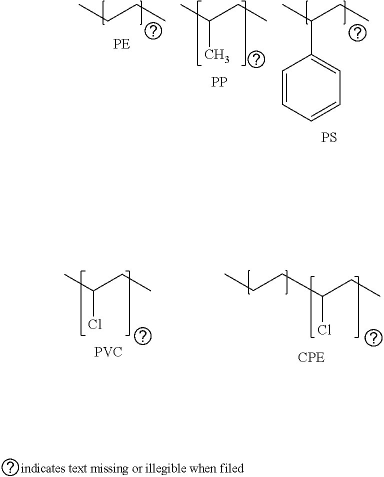

The biodegradation of these highly recalcitrant PA polymers has been recently reviewed by Danso et al. (2019) Applied and Environmental Microbiology 85: e01095-19. As explained by these authors, while many reports describing microbial communities degrading chemical additives have been published, no enzymes acting on the high-molecular-weight polymers polystyrene, polyvinylchloride, polypropylene and polyethylene are known (see Danso et al. (2019), Abstract).

Possible PE degradation has been linked with a large number of bacterial genera, including the genera Pseudomonas, Enterobacter, Stenotrophomonas, Rhodococcus, Staphylococcus, Streptomyces and Bacillus). Fungal genera that have been associated with PE degradation include Aspergillus, Cladosporium and Penicillium.

These studies used commercial polymers that possibly contained chemical additives (such as plasticisers), and degradation was determined by measuring weight loss and by Fourier transform infrared spectroscopy (FTIR). Since weight loss and surface structure changes are most likely attributed to the degradation of chemical additives, which often make up a significant fraction of the polymer, the results are unreliable.

Moreover, none of these studies revealed biochemical mechanisms and enzymes involved in PE breakdown (Danso et al., op. cit.). In this regard, various microbial enzymes capable of degrading lignin (a heterogeneous cross-linked phenolic polymer with oxidizable C—C bonds found in plant cell walls) have been linked with at least some level of degradative activity against PE. These include laccase (EC 1.10.3.2.), manganese peroxidase (MnP, EC 1.11.1.13) and lignin peroxidase (LiP, EC 1.11.1.14).

A laccase from Rhodococcus ruber C208 degraded UV-irradiated PE films both in culture supernatants and in cell-free extracts in the presence of copper (Santo et al. (2013) Int Biodeterior Biodegradation 84: 204-210), while another laccase (from Trametes versicolor) degraded a PE membrane in the presence of an oxidation mediator (1-hydroxybenzotriazole) (Fujisawa et al. (2001) J Polym Environ 9: 103-108).

The extracellular production of both laccase and MnP by Bacillus cereus was associated with the degradation of UV-irradiated PE (Sowmya et al. (2014) Adv Polym Sci Technol-Int J 4: 28-32). However, the incubation of similarly pre-treated PE with a partially purified laccase and a MnP from Penicillium simplicissimum resulted in a negligible weight loss (less than 1%—Sowmya et al. (2015) Environ Develop Sustain 17: 731-745). Ehara et al. (2000) J Wood Sci 46: 180-183 and liyoshi et al. (1998) J Wood Sci 44: 222-229 report PE degradation by manganese peroxidase partially purified from the fungus Phanerocheate chrysosporium.

Khatoon et al. (2019 Environmental Technology 40 (11): 1366-1375) report PVC degrading activity in a fungal lignin peroxidase partially purified from P. chrysosporium.

A purified hydroquinone peroxidase (EC 1.11.1.7) of the lignin-decolorizing Azotobacter beijerinckii degraded PS in a two-phase system consisting of dichloromethane and water (Nakamiya et al. (1997) J Ferm Bioeng 84: 480-482). However, this two-phase process has not been further developed for a recycling process.

However, the redox potential required for the oxidative breakdown of lignin is considerably lower than that for the homogenous C—C backbone of PE, and so efficient degradation of PE by the enzymes discussed above cannot be expected (Krueger et al. (2015) Appl. Microbiol. Biotechnol. 99: 8857-8874). Moreover, the above-mentioned studies used only culture supernatants or partially purified enzyme preparations and required long incubation times.

PE degradation by caterpillars of the wax moth Galleria mellonella has been reported (Bombelli (2017) Current Biology 27(8) R292-R293), and the degradation of both PE and PS by novel bacterial strains isolated from the guts of waxworm and mealworm larvae has also been observed (Yang et al. (2014) Environ Sci Technol 48: 13776-13784); Yang et al. (2015) Environm Sci Technol 49: 12087-12093). More recent reports in this area include Cassone et al. (2020) Proc R Soc B 287: 20200112; Lou et al. (2020) Environ Sci Technol 54: 2821-2831; and Zhang et al. (2020) Science of the Total Environment 704:135931). However, the enzymes involved have not been identified.

Linear paraffin molecules below a molecular weight of 500 Da (i.e. having a backbone length of less than 40 carbon atoms) are utilized by several microorganisms (Albertsson et al. (1987) Polymer Degradation and Stability 18: 73-87). The enzymatic basis for this activity may involve alkane hydroxylases (AH) of the AlkB family (EC 1.14.15.3), which can catalyse the degradation of hydrocarbon oligomers by terminal or subterminal oxidation (Rojo (2010) In Handbook of Hydrocarbon and Lipid Microbiology, Timmis, K. N. (ed). Berlin, Heidelberg: Springer-Verlag, 781-797).

It will therefore be appreciated that there remains a pressing need for alternative and/or improved enzymes capable of degrading PA polymers for use in bioremediation and in reactors for the degradation of plastic waste.

The present invention is based, at least in part, on the discovery of an unexpected polyalkenase activity within the KatG family of enzymes, and a finding that enzymes of the catalase-peroxidase (EC 1.11.1.21) class exhibit polyalkenase activity.

### SUMMARY OF THE INVENTION

According to a first aspect of the present invention, there is provided a composition for use in the degradation of a polyalkene (PA) polymer, comprising a KatG/EC 1.11.1.21 polyalkenase enzyme and a suitable excipient. Certain enzymes, particularly mutant forms of B2UBU5 are new and accordingly form an aspect of the invention. Thus the present invention provides enzyme variants of SEQ ID NO: 1 selected from the group: B2UBU5 KatG^{N}, enzymes having one or more of the following mutations M2481, M248V, Y221F, Y221A, Y221L, W98A, W98F and/or R410N. Enzymes having at least 50% identity to B2UBU5 or B2UBU5 KatG^{N}. Preferred is a B2UBU5 variant comprising aY221F and/or R410N mutation, more preferred is a B2UBU5 variant comprising both Y221F & R410N mutations.

According to a second aspect of the present invention, there is provided an enzyme composition for use in the degradation of a polyalkene (PA) polymer, the composition comprising a KatG/EC 1.11.1.21 PAase and one or more helper enzymes for generating hydrogen peroxide.

According to a third aspect of the present invention, there is provided a plasticase cocktail composition, the cocktail comprising a KatG/EC 1.11.1.21 PAase and one or more adjunctive plasticase enzymes.

According to a fourth aspect of the present invention, there is provided a waste remediation enzyme cocktail composition for degradation of a polyalkene (PA) polymer the cocktail comprising a KatG/EC 1.11.1.21 PAase and one or more ancillary non-plasticase bioremediation enzymes.

According to a fifth aspect of the present invention, there is provided a recombinant cellular host expressing KatG/EC1.11.1.21 PAase or variant thereof.

According to a sixth aspect of the present invention, there is provided a composition wherein the composition comprises a KatG/EC 1.11.1.21 PAase in combination with a PA polymer substrate.

According to a seventh aspect of the present invention, the composition of the invention is provided for use in the bioremediation of material contaminated with a PA polymer substrate.

According to an eighth aspect of the present invention, there is provided a KatG prodegradant comprising a KatG/EC 1.11.1.21 polyalkenase enzyme (and/or a microorganism expressing and/or secreting a KatG/EC 1.11.1.21 polyalkenase enzyme), for use as a biotic prodegradant plastic additive.

According to a ninth aspect of the present invention, there is provided a composite KatG plastic comprising a plastic polymer in combination with the KatG prodegradant of the invention.

According to a tenth aspect of the present invention, there is provided an autodegradative PA polymer comprising a PA polymer in combination with the KatG prodegradant of the invention.

According to an eleventh aspect of the present invention, there is provided a plastic article comprising the KatG prodegradant, composite KatG plastic or autodegradative PA polymer of the invention.

According to a twelfth aspect of the present invention, there is provided a method for the enzymatic degradation of a PA polymer, the method comprising the step of contacting said PA polymer with a composition comprising a PAase of the invention whereby C—C bonds in the PA polymer are cleaved, thereby enzymatically degrading the PA polymer.

According to a thirteenth aspect of the present invention, there is provided a process for producing fragments, oligomers, repeating unit fragments repeating unit derivatives of a PA polymer, the process comprising the step of degrading said polymer by contacting it with a composition comprising a PAase of the invention under conditions whereby C—C bonds in the PA polymer are cleaved, thereby enzymatically degrading the polymer. Derivatives include oxidised products of the form H—O—O—X (peroxides), HC(═O)—X (aldehydes), HO(O═)C—X (carboxylic acids), and the corresponding homo-di-substituted variants (di-peroxides, dialdehydes, and in particular di-carboxylic acids), and mixed hetero derivatives (mixed acids, aldehydes, alcohols and peroxides, mutatis mutandis) in which the degree of oxidation of the oxidised polyalkenes varies. Typical derivatives include oxidised C2-C20 polyalkanes such as oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc, dodecanedioic acids, glycolic acid, glyoxylic acid, citric acid, furoic and phenylpyruvic acids.

According to a fourteenth aspect of the present invention, there is provided the use of a composition comprising a KatG/EC 1.11.1.21 PAase for the degradation of a PA polymer.

According to an fifteenth aspect of the present invention, there is provided the use of a composition comprising a PAase for producing fragments, oligomers, repeating unit fragments repeating unit derivatives of a PA polymer, typically as described above.

According to a sixteenth aspect of the present invention, there is provided the use of a composition comprising a PAase for the bioremediation of material contaminated with a PA polymer.

According to a seventeenth aspect of the present invention, there is provided a process for producing microbial biomass comprising the steps of: (a) providing a seed culture comprising at least one microorganism expressing a KatG/EC 1.11.1.21 PAase; (b) providing a feedstock comprising a PA polymer; (c) mixing said seed culture with said feedstock to form a seeded feedstock; and (d) incubating the seeded feedstock whereby C—C bonds in the PA polymer are cleaved to produce degradation products of said polymer which are metabolized by said microorganism to yield said microbial biomass.

According to an eighteenth aspect of the present invention, there is provided a process for producing a bioproduct comprising the steps of: (a) providing a seed culture comprising at least one microorganism expressing a KatG/EC 1.11.1.21 PAase; (b) providing a feedstock comprising a PA polymer; (c) mixing said seed culture with said feedstock to form a seeded feedstock; and (d) incubating the seeded feedstock whereby C—C bonds in the PA polymer are cleaved by said enzyme to produce degradation products of said polymer which are metabolized by said microorganism to yield said bioproduct.

According to a nineteenth aspect of the present invention, there is provided a process for producing a hyperactive KatG enzyme comprising the steps of:
(a) providing a wild-type KatG enzyme having a first PAase activity;
(b) providing a plurality of mutant forms of said wild-type KatG enzyme which has one or more amino acid substitutions, deletions or insertions relative to the amino acid sequence of the wild-type KatG;
(c) determining the PAase activity of the mutant forms of step (b); and
(d) identifying a hyperactive KatG enzyme having a PAase activity which is higher than said first PAase activity.

According to a twentieth aspect of the present invention, there is provided assay kit for the high-throughput screening of KatG mutant forms comprising a microbead releasably coupled to a chromophore or fluorophore, which chromophore or fluorophore is released by PAase activity. Preferably microorganisms expressing said Kat G mutants are grown under selective pressure which favours the growth of cells expressing the KatG mutants or inhibits the growth of cells that do not express such mutants.

According to a twenty-first aspect there is provided a method for the production of dicarboxylic acids, the method comprising incubating a KatG enzyme a polyalkene (PA) polymer in the presence of hydrogen peroxide.

According to a twenty second aspect there is provided a method for the production of dicarboxylic acids, the method comprising incubating a KatG enzyme a polyalkene (PA) polymer in the presence of oxygen.

The invention further provides a reactor containing a KatG enzyme as herein described or KatG composition as herein described for the degradation of polyalkene polymers. The invention further provides a reactor for carrying out the process of polyalkene polymer degradation as described herein.

Other aspects, preferred features and preferred operative combinations of features of the invention are defined and described in the claims appended hereto.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### 1. Definitions and General Preferences

The term KatG enzyme is a term of art which defines a structural sub-class of phylogenetically-related enzymes which fall within Class I of the haem peroxidase-catalase (Px-Ct) superfamily (Passardi et al. Genomics 89 (2007) 567-579). As such, KatGs are distinct from two other sub-classes within Class I (the ascorbate peroxidases (APx, EC 1.11.1.11) and cytochrome c peroxidases (CcP, EC 1.11.1.5)). The KatG enzymes of the invention cleave C—C bonds in polyalkene (PA) polymer substrates, thereby enzymatically degrading the polymer into: (a) fragments; and/or (b) oligomers; and/or (c) isolated monomers or repeating units; and/or (d) monomer or repeating unit fragments; and/or (e) monomer or repeating unit derivatives. Preferred PA polymer substrates comprise polymer segments having a C—C backbone. In more detail, in the presence of oxygen or hydrogen peroxide the PA polymer substrate is typically degraded into C1-C12 dicarboxylic acids Preferred dicarboxylic acids produced include: oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids. Such compounds maybe purified and find utility in a range of applications known to the man skilled in the art.

Classes of use for such compounds as high value specialty chemicals are known to those skilled in the art, and include for a number of manufacturing processes such as in the electronics, flavor and fragrance, specialty solvents, polyesters, polymer cross-linking, food additives and pharmaceutical industries. By way of further example, malonic acid (and malonate; MA) is used as a building block chemical to produce diverse valuable compounds. Malonic acid and chemical derivatives of malonic acid (such as, for example, monoalkyl malonate, dialkyl malonate, and 2,2-dimethyl-1,3-dioxane-4,6-dione (“Meldrum's acid”)) are used for the production of many industrial and consumer products, including polyesters, protective coatings, solvents, electronic products, flavors, fragrances, pharmaceuticals, surgical adhesives, and food additives.

The term catalase-peroxidase EC 1.11.1.21 is a term of art which defines a functional sub-class of enzymes which exhibit dual peroxidase and catalase activity. The Enzyme Commission number (EC number) is a numerical classification scheme for enzymes, based on the chemical reactions they catalyze. Thus, EC numbers do not specify enzymes per se (i.e. do not necessarily define a homogeneous structural class), but rather enzyme-catalyzed reactions (i.e. the EC number defines a functional class). Thus, different enzymes which catalyze the same reaction receive the same EC number. Moreover, through convergent evolution, completely different protein folds may catalyze an identical reaction and so would be assigned an identical EC number (and dubbed non-homologous isofunctional enzymes, or NISE). Without wishing to be bound by any theory, it is believed that naturally-occurring catalase-peroxidase EC 1.11.1.21 enzymes are KatG enzymes (as defined above).

The present inventors have discovered that the catalase-peroxidase EC 1.11.1.21 enzymes of the invention cleave C—C bonds in polyalkene (PA) polymer substrates thereby enzymatically degrading the polymer. into: (a) fragments; and/or (b) oligomers; and/or (c) isolated monomers or repeating units; and/or (d) monomer or repeating unit fragments; and/or (e) monomer or repeating unit derivatives.

Such derivatives include dicarboxylic acid with C2-C12 backbone. The backbone may be saturated, partially saturated and maybe in straight or branched forms. For example oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids. Other degradation products include glycolic acid, glyoxylic acid, citric acid, furoic and phenylpyruvic acids.

Preferred PA polymer substrates comprise polymer segments having a C—C backbone.

As used herein, the term KatG/EC 1.11.1.21 polyalkenase enzyme defines a KatG enzyme (as herein defined) or a catalase-peroxidase EC 1.11.1.21 (as herein defined) which cleave C—C bonds in polyalkene (PA) polymer substrates, thereby enzymatically degrading the polymer into: (a) fragments; and/or (b) oligomers; and/or (e) monomer or repeating unit derivatives. For ease of reading, such KatG/EC 1.11.1.21 polyalkenase enzymes may be referenced herein as PAases of the invention (or when context permits, simply PAases), and they are to be understood as including the various natural and artificial KatG/EC 1.11.1.21 polyalkenase enzymes/enzyme variants described in Section 3 (below).

As used herein, the term polymer defines a chemical compound which comprises repeating units linked by covalent chemical bonds. The repeat units may be formed by the polymerization of monomers. Unless indicated otherwise by context, the term polymer encompasses both natural and synthetic polymers.

The linked polymerized monomers or repeating units may be of a single species (such polymers being referenced herein as homopolymers) or a mixture of two or more different monomers or repeating units (such polymers being referenced herein as hetero- or copolymers).

Copolymers comprising two different repeating units (typically obtained by copolymerization of two different monomer species) may be referenced as bipolymers, while those comprising three (typically obtained by copolymerization of three different monomer species) or four different repeating units (typically obtained by copolymerization of four different monomer species) may be referenced as terpolymers and quaterpolymers, respectively.

Copolymers may therefore contain two or more distinct backbone segments, depending on the organization of the repeating units.

Copolymers are further classified as random (statistical), alternating, block or graft copolymers (according to the arrangement of the repeating units).

Linear polymers consist of a single main chain (or backbone). Branched polymers (such as branched polyethylene and graft copolymers as a class), consist of a single main chain or backbone with one or more polymeric side chains. Branched polymers may have any configuration or architecture, including star, regular or irregular comb, brush, reticulated, networked and/or dendrimeric.

Polymers consisting of 2 to 50, and preferably 2-20, repeating units are herein referenced as oligomers. Polymers having a molecular weight of less than 2000 Da are herein referenced as fragments in circumstances where they are derived from a longer polymer by a degradative process (for example, enzymatic degradation).

As used herein, the term polyester in relation to certain synthetic plastic polymers defines those containing an ester functional group in their main chain. Esters are generally derived from a carboxylic acid and an alcohol. Polyester polymers can be aromatic, aliphatic or semi-aromatic.

As used herein, the term polyamide in relation to certain synthetic plastic polymers defines those comprising repeating units linked by amide bonds. Polyamide polymers can be aromatic, aliphatic or semi-aromatic.

In the context of polymer degradation, the terms fragmentation, oligomerization and depolymerisation are to be understood as the generation of: (a) fragments; and/or (b) oligomers; and/or (c) isolated monomers or repeating units, monomer or repeating unit fragments or monomer or repeating unit derivatives, respectively.

As used herein, the term polyalkenase (which may be abbreviated as PAase) an enzyme which cleaves C—C bonds in the backbone (or backbone segments) of a polyalkene (PA) polymer, whereby said PA polymer is enzymatically degraded by fragmentation, oligomerization and/or depolymerization. PA polymers which are degradable in this way by the PAases of the invention include one or more of the various PA polymers as defined and described in Section 4.2.1 (below). PA polymer substrates include various synthetic PA plastic substrates and PA plastic products as defined and described in Sections 4.3 and 4.4 (below). Thus, in preferred embodiments, the PAase of the invention cleaves C—C bonds in the backbone (or backbone segments) of a PA polymer, synthetic PA plastic substrate or PA plastic product, whereby said PA polymer, plastic or product is enzymatically degraded by fragmentation, oligomerization and/or depolymerization.

As used herein, the term polyethylenase (which may be abbreviated as PEase) defines a PAase (as defined above) which cleaves C—C bonds in the backbone (or backbone segments) of a polyethylene (PE) polymer, whereby said PE polymer is enzymatically degraded by fragmentation, oligomerization and/or depolymerization. PE polymers which are degradable in this way by the PEases of the invention include one or more of the various PE polymers as defined and described in Section 4.2.1 (below). PE polymer substrates include various synthetic PE plastic substrates and PE plastic products as defined and described in Sections 4.3 and 4.4 (below). Thus, in preferred embodiments, the PEase of the invention cleaves C—C bonds in the backbone (or backbone segments) of a PE polymer, synthetic PE plastic substrate or PE plastic product, whereby said PE polymer, plastic or product is enzymatically degraded by fragmentation, oligomerization and/or depolymerization.

As used herein, the term polypropylenase enzyme (which may be abbreviated as PPase) defines a PAase (as defined above) which cleaves C—C bonds in the backbone (or backbone segments) of a polypropylene (PP) polymer, whereby said PP polymer is enzymatically degraded by fragmentation, oligomerization and/or depolymerization. PP polymers which are degradable in this way by the PPases of the invention include one or more of the various PP polymers as defined and described in Section 4.2.2 (below). PP polymer substrates include various synthetic PP plastic substrates and PP plastic products as defined and described in Sections 4.3 and 4.4 (below). Thus, in preferred embodiments, the PPase of the invention cleaves C—C bonds in the backbone (or backbone segments) of a PP polymer, synthetic PP plastic substrate or PP plastic product, whereby said PP polymer, plastic or product is enzymatically degraded by fragmentation, oligomerization and/or depolymerization.

As used herein, the term polyvinyl chloridase enzyme (which may be abbreviated as PVCase) defines a PAase (as defined above) which cleaves C—C bonds in the backbone (or backbone segments) of a polyvinyl chloride (PVC) polymer, whereby said PVC polymer is enzymatically degraded by fragmentation, oligomerization and/or depolymerization. PVC polymers which are degradable in this way by the PVCases of the invention include one or more of the various PVC polymers as defined and described in Section 4.2.3 (below). PVC polymer substrates include various synthetic PVC plastic substrates and PVC plastic products as defined and described in Sections 4.3 and 4.4 (below). Thus, in preferred embodiments, the PVCase of the invention cleaves C—C bonds in the backbone (or backbone segments) of a PVC polymer, synthetic PVC plastic substrate or PVC plastic product, whereby said PVC polymer, plastic or product is enzymatically degraded by fragmentation, oligomerization and/or depolymerization.

As used herein, the term polystyrenase enzyme (which may be abbreviated as PSase) defines a PAase (as defined above) which cleaves C—C bonds in the backbone (or backbone segments) of a polystyrene (PS) polymer, whereby said PS polymer is enzymatically degraded by fragmentation, oligomerization and/or depolymerization. PS polymers which are degradable in this way by the PSases of the invention include one or more of the various PS polymers as defined and described in Section 4.2.4 (below). PS polymer substrates include various synthetic PS plastic substrates and PS plastic products as defined and described in Sections 4.3 and 4.4 (below). Thus, in preferred embodiments, the PSase of the invention cleaves C—C bonds in the backbone (or backbone segments) of a PS polymer, synthetic PS plastic substrate or PS plastic product, whereby said PS polymer, plastic or product is enzymatically degraded by fragmentation, oligomerization and/or depolymerization.

As used herein, the term chlorinated polyethylenase enzyme (which may be abbreviated as CPEase) defines a PAase (as defined above) which cleaves C—C bonds in the backbone (or backbone segments) of a chlorinated polyethylene (CPE) polymer, whereby said CPE polymer is enzymatically degraded by fragmentation, oligomerization and/or depolymerization. CPE polymers which are degradable in this way by the CPEases of the invention include one or more of the various CPE polymers as defined and described in Section 4.2.1 (below). CPE polymer substrates include various synthetic CPE plastic substrates and CPE plastic products as defined and described in Sections 4.3 and 4.4 (below). Thus, in preferred embodiments, the CPEase of the invention cleaves C—C bonds in the backbone (or backbone segments) of a CPE polymer, synthetic CPE plastic substrate or CPE plastic product, whereby said CPE polymer, plastic or product is enzymatically degraded by fragmentation, oligomerization and/or depolymerization.

As used herein, the term polyalkene (PA) polymer defines a polymer which comprises a C—C backbone (or backbone segment, in the case of PA copolymers) of repeating units (typically corresponding to polymerized alkene monomers).

Preferred PA polymers comprise polymer segments having a C—C backbone and include the various types of polyethylene (PE) and structurally related polyalkene polymers (which may be considered as substituted polyethylenes), as described in Section 4.2.1, below).

These preferred PA polymers therefore include various types of polyethylene (PE), polypropylene (PP), polyvinyl chloride (PVC), polystyrene (PS), chlorinated polyethylene (CPE), acrylonitrile-butadiene-styrene (ABS), polybutadiene (BR) and polyisoprene (IR), which comprise polymer tracts or segments having a C—C backbone, described in more detail in Section 4.2 (below), and which may be represented schematically as shown below:

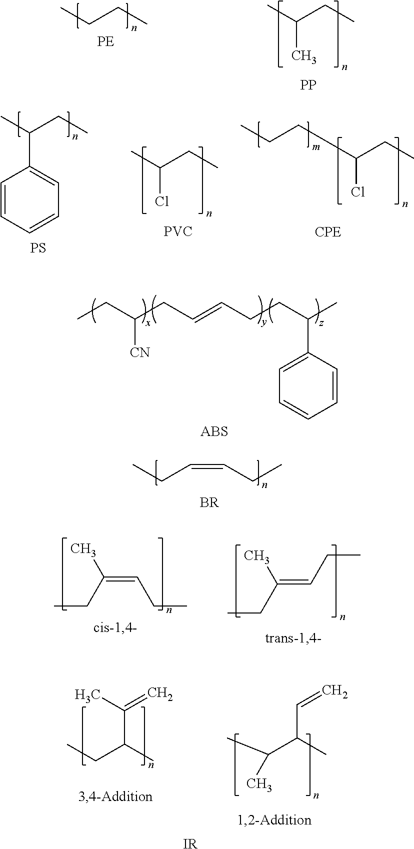

As used herein, the term synthetic polyalkene (PA) plastic defines a non-natural PA polymer. Such polymers may be referenced as polyolefins in the art, being synthesised from fossil fuels (including oil and natural gas). Synthetic PA plastics as herein defined have a molecular weight of at least 5000 Da, and more typically have a molecular weight of at least 20,000 Da. Synthetic PA plastics as herein defined also comprise crystalline, semi-crystalline and/or amorphous domains, in most cases comprising both crystalline (and/or or semi-crystalline) and amorphous domains. Synthetic PA plastics are typically solid thermoplastic materials at room temperature (though some chemically modified and/or copolymer synthetic PA plastics (e.g. PEX) may be thermoset plastics). The polyalkene backbone (or backbone segment, in the case of PA copolymers) may be linear or branched. Synthetic polyalkene plastics include those produced by bioprocessing, such as polyisoprene polymers produced using metabolic pathway engineering (see Section 4.2.7, below).

It will therefore be appreciated that natural polymers (such as certain waxes), which may comprise polyalkene tracts, domains or substructures, typically have much lower molecular weights, as do synthetic paraffin waxes and oils. Natural rubbers have higher molecular weights (around 10⁶ Da), but are chemically distinct from their synthetic isoprene rubber counterparts. The foregoing natural polymers are not synthetic plastic polyalkene (PA) plastics as herein defined.

As used herein, the term PA plastic product defines a plastic article comprising one or more PA polymers and/or synthetic PA plastics which has been formed into an article. The plastic article may be a moulded plastic article. It may be characterized by physical conformations selected from sheets, films, tubes, pipes, straws, rods, ropes, strings, lines, nets, reticulated sheets, three dimensional regular shapes, blocks, sheaths, fibres, membranes, woven and non-woven textiles, bags, containers, bottles, capsules, packaging, discs, microspheres, (electro)mechanical components, clothing, single-use coffee cups, single-use coffee capsules, building components, Lego® bricks, coatings, panels and moulded and extruded profiles.

Preferred synthetic PA plastics and PA plastic products comprise the various types of PA polymers described above, including polyethylene (PE), polypropylene (PP), polyvinyl chloride (PVC), polystyrene (PS), chlorinated polyethylene (CPE), acrylonitrile-butadiene-styrene (ABS), polybutadiene (BR) and polyisoprene (IR) polymers described above.

As used herein, the term plasticase is used as a generic term for any enzyme capable of enzymatically degrading any synthetic plastic polymer by fragmentation and/or depolymerisation thereof.

As used herein, the term KatG^{N} defines the catalytic N-terminal domain of the KatG enzyme.

As used herein, the term KatG^{C} defines the non-catalytic C-terminal domain of the KatG enzyme.

As used herein, the term KatG^{N} fusion defines a KatG enzyme variant which comprises a KatG^{N} domain fused to a heterologous KatG^{C} domain (or a synthetic analogue thereof).

As used herein, the term B2UBU5 defines the KatG enzyme of Ralstonia pickettii (strain 12J) (UniProt Accession Number: B2UBU5, entry name KATG_RALPJ, release 2020_01, entry version 76) having the sequence as set forth in SEQ ID NO: 1

As used herein, the term B2UBU5 KatG^{N} defines the catalytic N-terminal domain (aa's 77-391) of B2UBU5.

As used herein, the term B2UBU5 KatG^{C} defines the non-catalytic C-terminal domain (aa's 398-691) of B2UBU5.

As used herein, the term orthologue in relation to a protein defines a species variant of that protein which has diverged in different organisms following a speciation event. Orthologues may perform the same, or a different, role in each organism in which they are found.

As used herein, the term B2UBU5 orthologue defines a KatG enzyme which is homologous to B2UBU5 (i.e. which appear to share a common ancestor on the basis of phylogenetic analyses involving sequence comparisons), but which is derived from a different organism (i.e. it is a species or strain variant which may have structurally and/or functionally diverged following a speciation event).

Preferred B2UBU5 orthologues are KatG enzymes from a Ralstonia spp/strain other than Ralstonia pickettii (strain 12J), more preferably from a Ralstonia species/strain selected from: Ralstonia sp. NFACC01; Burkholderiaceae bacterium 26; Ralstonia sp. MD27; Ralstonia sp. AU12-08; Ralstonia insidiosa; Ralstonia sp. TCR112; Ralstonia pickettii (strain 12D); Ralstonia pickettii (Burkholderia pickettii); Ralstonia sp. 5_2_56 FAA; Ralstonia pickettii OR214; Ralstonia mannitolilytica; Ralstonia sp. SET104; Ralstonia sp. GX3-BWBA; Ralstonia sp. NT80; Ralstonia sp. GV074; and Ralstonia sp. UNC404CL21Col.

As used herein, the term hyperactive KatG enzyme is used in relation to B2UBU5, B2UBU5 KatG^{N} and B2UBU5 orthologues to define variants which comprise, consist of, or consist essentially of, an amino acid sequence which is at least 50%, 55%, 60%, 65%, 70%, 75%, 80%, 85%, 90%, 95% or 99% identical to the amino acid sequence of B2UBU5, B2UBU5 KatG^{N} or a B2UBU5 orthologue (including in particular the preferred B2UBU5 orthologues listed above) and which have one or more amino acid substitutions, deletions or insertions which increase polyalkenase activity relative to B2UBU5.

As used herein, the term M1FBG9 defines the KatG enzyme of the dinoflagellate Prorocentrum minimum (UniProt Accession Number: M1FBG9, entry name M1FBG9_PROMN, release 2019_11, entry version 16).

As used herein, the term identity in relation to an amino acid sequence defines the number (or fraction when expressed as a percentage, %) of identical amino acid residues between two polypeptide sequences. The sequence identity is determined by comparing the sequences when aligned so as to maximize overlap and identity while minimizing sequence gaps, and may be determined using any of a number of alignment algorithms known to those skilled in the art. In particular, sequence identity may be determined using the public domain BLAST (2.10.0) program.

As used herein, the term KatG prodegradant is used to define a composition comprising a KatG/EC 1.11.1.21 polyalkenase enzyme of the invention (and/or a microorganism expressing and/or secreting the KatG/EC 1.11.1.21 polyalkenase enzyme), in a form suitable for use as a prodegradant plastic additive or coating.

As used herein, the term recombinant is used to define material which has been produced by that body of techniques collectively known as “recombinant DNA technology” (for example, using the nucleic acid, vectors and or host cells described infra).

As used herein, the term room temperature (r.t.) defines a temperature of 20° C.

As used herein, the term aa refers to amino acids.

### 2. Reactors for Use According to the Invention

### 2.1 General Considerations

The reactors for use according to the invention comprise one or more reactor vessels for containing a reaction mix comprising the PAase of the invention (or microorganism(s) expressing it) and a PAase polymer substrate.

Further details of the PAase and PAase polymer substrate are set out below (in Sections 3 and 4, respectively).

The reactors for use according to the invention may also comprise a source of peroxide (for example a source of hydrogen peroxide) or means for generating peroxide (for example means for generating hydrogen peroxide). Any suitable (hydrogen) peroxide source or generating means may be employed, including electrochemical generators. Such generators may for example be situated within or adjacent to the reactor, for example for producing hydrogen peroxide within (or for supplying hydrogen peroxide to) the reactor vessel(s).

In preferred embodiments, the source of peroxide comprises one or more helper enzymes which produce peroxide within the reactor vessel(s) in conjunction with suitable substrates, water and a source of oxygen (such as compressed air).

The present invention may be practised without the provision of peroxide, provided oxygen is present.

The reactors for use according to the invention preferably further comprise a heating and/or a cooling coil, and may also further comprise means for monitoring and controlling the pH of the reaction mix.

The reactors for use according to the invention preferably further comprise an aerator and/or a stirrer.

The reactors for use according to the invention preferably further comprise means for sampling the reaction mix during operation, and preferably further comprise means for monitoring the degradation of the polymer. Suitable monitoring means include one or more instruments for analysing the chemical, electrochemical, morphological, rheological, gravimetric, spectroscopic and/or chromatographic characteristics of the reaction mix.

The reactors for use according to the invention may be adapted for use in the bioremediation of material contaminated with a PA polymer substrate. The contaminated material may be selected from soil, subsoil, marine sediment, freshwater sediment, groundwater, freshwater, seawater, drinking water and leachate.

Particularly preferred are airlift reactors (as described in more detail in Section 2.4.1, below).

### 2.2 Bioreactors

The reactors for use according to the invention may be bioreactors. As used herein, the term bioreactor defines a reactor in which the reaction mix comprises cells expressing and secreting a KatG/EC 1.11.1.21 polyalkenase enzyme (PAase) of the invention. The cells may comprise a cellular KatG/EC 1.11.1.21 polyalkenase (PAase) expression system (as herein defined).

The cells may also express one or more of helper enzymes (as herein defined), and/or one or more adjunctive plasticase enzymes (as herein defined) and/or one or more ancillary non-plasticase bioremediation enzymes (as herein defined). In cases where the cells express one or more of helper enzymes, adjunctive plasticases and/or ancillary non-plasticase bioremediation enzymes, the enzymes may be co-expressed in a single recombinant host cell.

Any suitable expression system may be used in the bioreactors of the invention, including those involving prokaryotic cells and eukaryotic cells. Suitable methodologies have been recently reviewed by Gomes et al. (2016) Advances in Animal and Veterinary Sciences 4(4): 346-356.

Suitable prokaryotic cells may be selected from: (a) Escherichia coli; (b) Bacillus subtilis; (c) Bacillus megaterium; (d) Bacillus licheniformis; (e) Lactococcus lactis; (f) Ralstonia eutropha; (g) Pseudomonas spp.; (h) Ralstonia pickettii; and (i) Corynebacterium spp.

Particularly preferred as a heterologous host are E. coli strains engineered to export the PAase to the cell surface (or extracellularly) using the AIDA-I autotransporter (by control of the the status of the protease ompT (+/−)), as described in Fleetwood et al. (2014) Microb Cell Fact 13:179; Jose and Meyer (2007) Microbiol Mol Biol Rev 71: 600-619.

Suitable eukaryotic cells may be selected from: (a) yeasts; (b) fungi; (c) insect cells; (d) mammalian cells; and (e) plant cells. Suitable yeast cells may be selected from: (a) Saccharomyces cerevisiae; (b) Hansenula polymorpha; (c) Komagatella phaffii; (d) Candida biodini; and (e) Schizosaccharomyces pombe. Suitable fungal cells may be selected from: (a) a filamentous fungus, for example Aspergillus spp. and Trichoderma spp., and (b) Myceliophthora thermophila (e.g. the C1 expression system).

The bioreactor of the invention may also contain one or more microorganisms which individually, or as part of a microbial consortium, degrade one or more plastic polymers or pollutants. Exemplary microorganisms are described in more detail in Section 5.3.4 (below).

2.3 Enzyme Reactors

The reactors for use according to the invention are preferably enzyme reactors. As used herein, the term enzyme reactor defines a reactor in which the KatG/EC 1.11.1.21 polyalkenase enzyme is not produced in the reactor vessel by a biological cell or cellular expression system, being present in a free form (or immobilized in or on one or more reactor components).

The enzyme reactors of the invention preferably further comprise one or more of helper enzymes (as herein defined), and/or one or more adjunctive plasticase enzymes (as herein defined) and/or one or more ancillary non-plasticase bioremediation enzymes (as herein defined). These additional enzymes may be present in a free form (or immobilized in or on one or more reactor components). Alternatively (or in addition), one or more of these additional enzymes may be produced in the enzyme reactor vessel by a biological cell or cellular expression system.

It will therefore be understood that the enzyme reactors of the invention may include biological cells expressing enzymes other than the KatG/EC 1.11.1.21 polyalkenase enzyme of the invention, including those described above in Section 2.2, provided that the KatG/EC 1.11.1.21 polyalkenase enzyme is not itself produced in the reactor vessel by a biological cell or cellular expression system. Suitable microorganisms are described in more detail in Section 5.3.3 (below).

### 2.4 Reactor Configurations

The reactors for use according to the invention may be batch or continuous reactors, and those skilled in the art will be able to select an appropriate reactor type and configuration taking into account processing volumes, throughput requirements, costs and nature of the plastic feedstock.

Suitable reactor configurations therefore include the following: stirred tank batch reactors (STR) (where the tank contains all of the enzyme and substrates and where the tank is stirred until the conversion is complete); batch membrane reactors (MR), where the enzyme is held within membrane tubes which allow the substrate to diffuse in and the product to diffuse out (which may be operated in a semi-continuous manner, using the same enzyme solution for several batches); packed bed reactors (PBR), also called plug-flow reactor (PFR), containing a settled bed of immobilised enzyme particles; continuous flow stirred tank reactors (CSTR) which is a continuously operated version of an STR; continuous flow membrane reactors (CMR) which is a continuously operated version of an MR and fluidised bed reactors (FBR), where the flow of gas and/or substrate keeps the immobilised enzyme particles in a fluidised state.

### 2.4.1 Airlift Reactors

Preferred reactors for use according to the invention are airlift reactors.

Airlift reactors are reactors (which may be enzyme reactors or bioreactors as defined and described above) in which the reaction medium is kept mixed and gassed by introduction of air or another gas (or mixture of gases) at the base of a column-like reactor element equipped either with a draught tube or another device (e.g. external tube) by which the reactor volume is separated into gassed and un-gassed (or relatively less gassed) regions, thus generating a vertically circulating flow. The airlift reactors for use according to the invention therefore include both internal and external loop airlift reactors.

Airlift reactors may be constructed with stainless steel and the draft tube in particular is advantageously made of stainless steel, typically cylindrically shaped and supported in a vertical position by arms connected to the inside of the bioreactor vessel. The air inlet is generally positioned just below the draft tube, so that the gas-liquid mixture rises inside the draft tube, although the opposite circulation mode is also possible when the air inlet is positioned outside the draught tube. The simplicity of the reactor and its geometrical relationship with the draft tube makes it possible to scale-up without difficulties to sizes of 1.000,000 litres or more.

### 3. KatG/EC 1.11.1.21 Polyalkenase Enzymes for Use According to the Invention

Any KatG/EC 1.11.1.21 polyalkenase enzyme (PAase) having polyalkenase activity may be used according to the invention, including the enzymes and enzyme classes exemplified and described in more detail below.

The PAase is preferably present in the compositions in isolated or purified form. For instance, the PAase may be expressed, derived, secreted, isolated, or purified from a microorganism (for example from Ralstonia pickettii). For example, the type strain ATCC 27511 may be used as a source of PAase for use according to the invention. The PAase may be purified by biochemical techniques known in the art.

The PAase may also be conveniently produced by recombinant techniques using a cellular expression system (see e.g. Johnsson et al. (1997) J Biol Chem 272: 2834-2840; Zámocký et al. (2012) Biochimie 94: 673-683; Doyle and Smith (1996) Biochem J 315 (Pt 1): 15-19).

Any suitable expression system may be used, including those involving prokaryotic cells and eukaryotic cells. Suitable methodologies have been recently reviewed by Gomes et al. (2016) Advances in Animal and Veterinary Sciences 4(4): 346-356. This approach is explained in more detail in Section 6.1 (below).

The PAase may be a naturally-occurring enzyme, or may be a variant of a naturally-occurring enzyme (as explained below), and so the PAases of the invention are to be understood as including the various natural and artificial KatG/EC 1.11.1.21 polyalkenase enzymes/enzyme variants described below.

### 3.1 KatG Enzymes

KatG enzymes are widely (but not universally) distributed among archaea, eubacteria, and lower eukaryotes, and across these species there are cytoplasmic and periplasmic/extracellular isoforms (some organisms express both a cytoplasmic and an extracellular KatG). Despite this wide distribution, there is little sequence diversity among KatGs from different sources and multiple sequence alignments show that these are very closely related enzymes despite their diverse origins (Passardi et al. Gene, 397 (2007) 101-113).

KatGs in their wild-type forms are typically multimeric (homodimers or homotetramers), and in most cases the KatG subunit is a two domain structure (with distinct N-terminal and C-terminal domains where each domain has a typical peroxidase scaffold). The two domain structure of KatG is unique in the Px-Ct superfamily (even among Class I enzymes), and appears to be the result of a gene-duplication and fusion event.

All known KatG enzymes in their wild-type forms are members of the catalase-peroxidase EC 1.11.1.21 class of enzymes. However, as explained below, the invention contemplates various KatG enzymes which are non-catalatic (as defined herein). Accordingly these variants form an aspect of the invention.

The present inventors have discovered that KatG enzymes may act as polyalkenases, an unexpected finding in the light of the known substrates of this class of enzymes (Gumiero et al. (2010) Arch Biochem Biophys 500: 13-20).

### 3.2 B2UBU5

B2UBU5 is a 721 aa protein comprising two domains, each homologous to haem peroxidases: an N-terminal domain (77-391) and a C-terminal domain (398-691). The former is herein referenced as B2UBU5 KatG^{N} and the latter as B2UBU5 KatG^{C}.

B2UBU5
(SEQ ID NO: 1)
MTTEAKCPFSGHAPAASHAFGGGTANKDWWPNQLRVDLLNQHSEKSDPL
GSNFNYRKSFNAIDYDALKADLRRLMTDSQDWWPADFGHYGPQFIRMAW
HAAGTYRTGDGRGGAGRGQQRFAPLNSWPDNVNIDKSRRLLWPIKQKYG
QAISWADLLILTGNVALETMGFRTFGFAAGREDTWEPDNDVYWGNETKW
LEATRYSGERNLANPLAAVQMGLIYVNPEGPEHAHGDPLAAAKDIRETF
ARMAMDDEETVALIAGGHTFGKTHGAGPASHVGADVEAAPLEAQGLGWA
STFGTGKGADAITSGLEVTWTQTPAQWSNFFFENLFKYEWVQEKSPAGA
LQWVAKDAEAIIPGPTPDSPKRRPTMLTTDLSLRFDPAYEKISRRFLDN
PQAFAEAFARAWFKLTHRDLGPKSRYLGPEVPREDLIWQDPLPTATHKP
TEADIADLKAKIAASGLSASELVAVAWASASTFRGSDKRGGANGARIRL
APQKDWAVNQPVAGTLARLEEIQRASGKASLADVIVLAGSVGIELAAKA
AGTTITVPFTPGRVDATAEQTDATSFSVLEPVADGFRNYQKTQFAVPGE
VLLLDRAQLLTLTAPELTVLIGGLRAININADGSQHGVFTSTPGALTND
FFVNLLDMNTEWKPAGDIYEGYDRKTGERKWTGTRVDLVFGSNSILRAL
AEVFGSADGKERFISEFVAAWVKVMNLDRFDLAAA

### 3.2.1 B2UBU5 and Variants

Preferred B2UBU5 enzymes for use according to the invention comprise, consist of, or consist essentially of, an amino acid sequence which is at least 50%, 55%, 60%, 65%, 70%, 75%, 80%, 85%, 90%, 95%, 99% or 100% identical to B2UBU5 KatG^{N}. B2UBU5 variants which consist of, or consist essentially of, an amino acid sequence which is at least 50%, 55%, 60%, 65%, 70%, 75%, 80%, 85%, 90%, 95%, 99% or 100% identical to B2UBU5 KatG^{N} may be KatG^{N} truncates, as described below.

Particularly preferred B2UBU5 enzymes for use according to the invention comprise, consist of, or consist essentially of, an amino acid sequence which is at least 50%, 55%, 60%, 65%, 70%, 75%, 80%, 85%, 90%, 95%, 99% or 100% identical to B2UBU5.

More particularly preferred are B2UBU5 variants which comprise, consist of, or consist essentially of, an amino acid sequence which is at least 50%, 55%, 60%, 65%, 70%, 75%, 80%, 85%, 90%, 95% or 99% identical to either B2UBU5 or to B2UBU5 KatG^{N} and which have one or more amino acid substitutions, deletions or insertions which:
(a) increase the polyalkenase activity; and/or
(b) increase thermostability; and/or
(c) increase psychrophilicity; and/or
(d) increase stability in relation to organic solvents, contaminants, detergents, pH and/or oxidants (including hydrogen peroxide).

Such variants can be produced by methods that are well-established in the art, including directed evolution and site-directed mutagenesis (see Zeymer and Hilvert (2018) Annual Review of Biochemistry 87:131-157; Watkins et al. Nat Commun 2017; 8:358; Watanabe and Nakajima, Methods Enzymol 2016; 580:455-470; Lichtenstein et al. Biochem Soc Trans 2012; 40:561-566; Anderson et al. Chem Sci 2014; 5:507-514; and Grayson and Anderson, Curr Opin Struct Biol 2018; 51:149-155.

### 3.2.2 B2UBU5 Orthologues (https://www.uniprot.org/uniprot/B2UBU5)

B2UBU5 orthologues having at least 90% amino acid sequence identity to B2UBU5 and suitable for use according to the invention include the following KatG enzymes (UniProt Accession Number followed by the entry name, if allocated, in parentheses):
A0A115SK77 (A0A1I5SK77_9 RALS)
A0A0F0E7J1 (A0A0F0E7J1_9 BURK)
A0A0J9DTW2 (A0A0J9DTW2_9 RALS)
S9RSJ5 (S9RSJ5_9 RALS)
A0A191ZZS8 (A0A191ZZS8_9 RALS)
A0A5C6Y6C3 (A0A5C6Y6C3-9RALS)
C6BD71 (C6BD71_RALP1)
A0A2P4RN88 (A0A2P4RN88_RALPI)
U3GIS7 (U3GIS7_9 RALS)
A0A1COXEH7 (A0A1COXEH7_RALPI)
R0CJV6 (R0CJV6_RALPI)
A0A2N4TPC1 (A0A2N4TPC1_RALPI)
A0A2N5LD46 (A0A2N5LD46_9 RALS)
A0A291E192 (A0A291E192_RALPI)
A0A401K9C7 (A0A401K9C7_9RALS)
UPI00046A45B6
UPI000DD31867
UPI000CEF361E
UPI00073F3865
UPI00046AAE20
UPI0012AE63A9
UPI000D5F3903
UPI0004803C34
UPI000CEDCA44

Preferred B2UBU5 orthologues for use according to the invention comprise, consist of, or consist essentially of, an amino acid sequence which is at least 50%, 55%, 60%, 65%, 70%, 75%, 80%, 85%, 90%, 95%, 99% or 100% identical to any one of the above KatG enzymes.

Also contemplated for use according to the invention are B2UBU5 orthologue variants which comprise, consist of, or consist essentially of, an amino acid sequence which is at least 50%, 55%, 60%, 65%, 70%, 75%, 80%, 85%, 90%, 95% or 99% identical to any one of the above KatG enzymes and which have one or more amino acid substitutions, deletions or insertions which:
(a) increase the polyalkenase activity; and/or
(b) increase thermostability; and/or
(c) increase psychrophilicity; and/or
(d) increase stability in relation to organic solvents, contaminants, detergents, pH and/or oxidants (including hydrogen peroxide).

As explained above, such variants can be produced by methods that are well-established in the art.

### 3.2.3 Non-Catalatic KatG Enzymes

Native KatG enzymes are post-translationally modified by an autocatalytic crosslinking event which yields a crosslinked Met-Tyr-Trp (MYW) tripeptide (also known as the tripeptide adduct or cofactor). The tripeptide adduct is located in, or near, the active site of the protein opposite the prosthetic haem iron atom. In the absence of the intact cross-linked tripeptide, KatG catalase activity is greatly attenuated, while the peroxidase activity may be increased (see Table 1, below, adapted from Paul R. Ortiz de Montellano, Chapter 1: Self-processing of Peroxidases, in Heme Peroxidases, 2015, pp. 1-30).

TABLE 1
Catalase and peroxidase activities of KatG mutants that result in incomplete
formation of the cross-linked tripeptide

| | | Catalase | Peroxidase |
|---|---|---|---|
| Organism | Mutation | (% of WT) | (% of WT) |
| B. pseudomallei | Tyr238Ala | 0.05 | 139 |
| B. pseudomallei | Tyr238Phe | 0.15 | 64 |
| Synechocystis | Tyr249Phe | 0.17 | 121 |
| M. tuberculosis | Tyr229Phe | 0.002 | 1360 |
| | | 0.07 | 311 |
| Sinorhizobium meliloti | Tyr217Leu | Not detected | 263 |
| Sinorhizobium meliloti | Tyr217Phe | Not detected | 358 |
| Sinorhizobium meliloti | Trp95Ala | Not detected | 116 |
| Sinorhizobium meliloti | Trp95Phe | Not detected | 321 |
| M. tuberculosis | Trpl07Phe | 0.07 | 333 |
| B. pseudomallei | Met264Ala | 0.15 | 160 |
| B. pseudomallei | Met264Leu | 0.02 | 140 |
| Synechocystis | Met275Ile | 0.2 | 120 |
| Sinorhizobium meliloti | Met243Val | 0.6 | 640 |
| | | Not detected | 248 |

Thus, the catalase and peroxidase activities of KatG/EC 1.11.1.21 polyalkenase enzymes can be separated, and non-catalatic variants can be derived (see e.g. Ghiladi et al. (2005) J Biol Chem 280: 22651-22663; Ghiladi et al. (2005) Biochemistry 44: 15093-15105; Vlasits et al. (2010) J Inorg Biochem 104: 648-656; Vlasits et al. (2010) Biochim Biophys Acta 2010b; 1804:799-805; Ortiz de Montellano (2010) Biocatalysis based on heme peroxidases: 79-107; Jakopitsch et al. (2003) J Biol Chem 278: 20185-20191; Jakopitsch et al. (2004) J Biol Chem 279: 46082-46095; Jakopitsch et al. (2003) FEBS Lett 552: 135-140; Santoni et al. (2004) Biopolymers 74: 46-50; Yu et al. (2003) J Biol Chem 278: 44121-44127; and Bernroitner et al. (2009) J Exp Bot 60: 423-440).

As used herein, the term non-catalatic, as applied in relation to both KatG enzymes and catalase-peroxidase EC 1.11.1.21 enzymes, defines an enzyme in which the catalase activity is reduced or eliminated but which retains polyalkenase activity. Preferred non-catalatic PAases contain one or more substitutions of the MYW tripeptide residues.

Other preferred non-catalatic PAases contain amino acid substitutions in amino acids other than the MYW residues (including those which impair the function of the MYW tripeptide). For example, substitution of the arginine residue corresponding to R439 of the Synechocystis KatG (R410) may also be employed to generate non-catalatic variants (see for example Jakopitsch et al. (2004) J Biol Chem 279: 46082-46095; Vlasits et al. (2010) 104: 648-656; Carpena et al. (2005) EM BO Rep 2005, 6:1156-1162).

Such non-catalatic KatG/catalase-peroxidase EC 1.11.1.21 enzymes may exhibit improved polyalkenase activity arising from the retained peroxidase activity. Thus, in preferred embodiments, non-catalatic KatG/catalase-peroxidase EC 1.11.1.21 enzymes exhibit improved polyalkenase activity arising from the retained peroxidase activity.

Thus, as used herein, the term catalase-peroxidase EC 1.11.1.21 may be used sensu lato to define a functional class of enzymes derived from enzymes in EC 1.11.1.21, but which exhibit reduced (or eliminated) catalase activity (i.e. are non-catalatic, as defined above) but which exhibit polyalkenase activity arising from the retained peroxidase activity.

Thus, preferred non-catalatic PAases contain substitutions of one or more of the MYW residues (and/or other mutations which disrupt, or impair the function of, the MYW tripeptide). The PAase for use according to the invention may therefore be a KatG variant in which the function of the MYW tripeptide (or the tripeptide itself) is disrupted such that catalase activity is reduced or eliminated. The MYW tripeptide may be disrupted by any means, including via the substitution of one or more of the constituent M, Y and/or W amino acids by site-directed mutagenesis.

In the case of B2UBU5, the MYW tripeptide residues are M248, Y221 and W98. Suitable substitutions of B2UBU5 therefore include variants comprising one or more of the following amino acid substitutions: M248I, M248V, Y221F, Y221A, Y221L, W98A, W98F and/or R410N. Preferred is a B2UBU5 variant comprising a Y221F and/or R410N substitution, particularly B2UBU5 variants comprising Y221F and R410N substitutions.

In preferred embodiments, the non-catalatic PAase is a variant of B2UBU5 or of a B2UBU5 orthologue, as defined above.

The above principles apply mutatis mutandis to catalase-peroxidase EC 1.11.1.21 enzymes (which, in their native form, also exhibit the MYW tripeptide), and such non-catalatic catalase-peroxidase EC 1.11.1.21 enzyme variants may exhibit improved polyalkenase activity.

### 3.2.4 KatG^{N} Truncates

KatG enzymes comprise two peroxidase-like domains. The N-terminal domain (KatG^{N}) contains the haem group and is catalytically active. The C-terminal domain (KatG^{C}) lacks the haem cofactor, has no catalytic activity, and is separated from the active site by >30 Å. KatG^{C} appears to contain a vestigial “active site” in which an Arg residue replaces what should be the His ligand, and haem binding is further precluded by a large hydrophobic side chain in the adjacent amino acid (Leu or Met) which protrudes into the haem binding cavity.

However, KatG^{C} is typically not redundant (the only reported KatG lacking the C-terminal domain is that from the dinoflagellate Prorocentrum minimum—see Guo and Ki, J Phycol. (2013) 49(5):1011-6). KatG truncates containing only the KatG^{N} domain were constructed and shown to exhibit neither catalase nor peroxidase activity. However, the isolated KatG^{N} domain retained gross secondary structure, and both activities were restored by adding back separately expressed and isolated KatG^{C} (Baker et al. Biochem Biophys Res Commun. 2004, 320(3): 833-9; Baker et al. Biochemistry 2006, 45(23): 7113-7121).

Thus, KatG^{C} functions as a platform for the folding of the N-terminal domain and as a scaffold for stabilization of the molecular dimer which can be supplied in trans as a separate moiety.

The invention therefore contemplates the use of KatG enzyme variants which consist, or consist essentially, of a KatG^{N} domain. Such variants are referenced herein as KatG^{N} truncates. In such embodiments, the KatG^{N} truncate may be used alone or together with separate KatG^{C} (or synthetic analogues thereof).

### 3.2.5 KatG^{N} Fusions

Also contemplated is the use of KatG enzyme variants which comprise a KatG^{N} domain fused to a heterologous KatG^{C} (or a synthetic analogue thereof). Such variants are referenced herein as KatG^{N} fusions.

Suitable synthetic analogues of KatG^{C} for use in combination with KatG^{N} truncates (or as part of a KatG^{N} fusion protein) may be produced by synbio methods that are well-established in the art (see e.g. Watkins et al. Nat Commun 2017; 8:358; Watanabe and Nakajima, Methods Enzymol 2016; 580:455-470; Lichtenstein et al. Biochem Soc Trans 2012; 40:561-566; Anderson et al. Chem Sci 2014; 5:507-514; and Grayson and Anderson, Curr Opin Struct Biol 2018; 51:149-155).

### 3.2.6 Hyperactive and Hyperstable KatG Enzymes

The invention contemplates hyperactive KatG enzymes which comprise, consist of, or consist essentially of, an amino acid sequence which is at least 50%, 55%, 60%, 65%, 70%, 75%, 80%, 85%, 90%, 95% or 99% identical to either B2UBU5 or to B2UBU5 KatG^{N} and which have one or more amino acid substitutions, deletions or insertions which increase polyalkenase activity relative to B2UBU5. Preferred hyperactive KatG enzymes are B2UBU5 variants which comprise, consist of, or consist essentially of, an amino acid sequence which is at least 50%, 55%, 60%, 65%, 70%, 75%, 80%, 85%, 90%, 95% or 99% identical to B2UBU5 and which have one or more amino acid substitutions, deletions or insertions which increase polyalkenase activity relative to B2UBU5.

The invention also contemplates hyperstable KatG enzymes which comprise, consist of, or consist essentially of, an amino acid sequence which is at least 50%, 55%, 60%, 65%, 70%, 75%, 80%, 85%, 90%, 95% or 99% identical to either B2UBU5 or to B2UBU5 KatG^{N} and which have one or more amino acid substitutions, deletions or insertions which increase stability relative to B2UBU5. Preferred hyperstable KatG enzymes are B2UBU5 variants which comprise, consist of, or consist essentially of, an amino acid sequence which is at least 50%, 55%, 60%, 65%, 70%, 75%, 80%, 85%, 90%, 95% or 99% identical to B2UBU5 and which have one or more amino acid substitutions, deletions or insertions which increase stability relative to B2UBU5.

In preferred embodiments, the hyperactive B2UBU5 variants of the invention preferably also have one or more amino acid substitutions, deletions or insertions which: (a) increase the polyalkenase activity; and/or (b) increase thermostability; and/or (c) increase psychrophilicity; and/or (d) increase stability in relation to organic solvents, contaminants, detergents, pH and/or oxidants (including hydrogen peroxide).

The term “increased thermostability” indicates an increased ability of a PAase enzyme of the invention to resist to changes in its chemical and/or physical structure at high temperatures, and particularly at temperature between 50° C. and 90° C., relative to B2UBU5.

The term “increased psychrophilicity” indicates an increased ability of a PAase enzyme of the invention to exhibit PAase activity at low temperatures, and particularly at temperatures below 20° C., below 15° C., below 10° C. or below 5° C., relative to B2UBU5.

Thermostability can be determined by measurement of the melting temperature (Tm) of the PAase. In this context, the “melting temperature” refers to the temperature at which half of the enzyme population assayed is unfolded or misfolded. The melting temperature (Tm) of a PAase may be measured according to methods known in the art. For example, differential scanning fluorimetry (DSF) may be used to quantify the change in thermal denaturation temperature of the PAase.

Alternatively, the Tm can be assessed by analysis of the protein folding using circular dichroism. Alternatively, the thermostability may be evaluated by measuring the PAase activity after incubation at different temperatures and comparing with the activity of B2UBU5 under the conditions selected.

It will be appreciated that such techniques may also be used to determine relative psychrophilicity and stability in relation to organic solvents, contaminants, detergents, pH and/or oxidants (including in particular hydrogen peroxide).

The above KatG enzymes can be produced by methods that are well-established in the art, including directed evolution and site-directed mutagenesis (see Zeymer and Hilvert (2018) Annual Review of Biochemistry 87:131-157; Watkins et al. Nat Commun 2017; 8:358; Watanabe and Nakajima, Methods Enzymol 2016; 580:455-470; Lichtenstein et al. Biochem Soc Trans 2012; 40:561-566; Anderson et al. Chem Sci 2014; 5:507-514; Grayson and Anderson, Curr Opin Struct Biol 2018; 51:149-155; Currin et al. (2019) ACS Synth Biol 8: 1371-1378; Currin et al. (2014) Protein Eng Design Sel 27: 273-280; Currin et al. (2017) Meth Mol Biol 1472: 63-78; Currin et al. (2015) Chem Soc Rev 44: 1172-1239).

Hyperactivity (i.e. increased polyalkenase activity relative to B2UBU5) can be identified in any suitable assay, including the high-throughput screening assays of the invention.

### 3.2.7 KatG-PBP Fusions

Also contemplated is the use of KatG enzyme variants which comprise a KatG enzyme (as described in any one of Sections 3.1-3.2.6, above), or a KatG^{N} domain, fused to a plastic binding protein. Such variants are referenced herein as KatG-PBP fusions.

The term plastic binding protein is used herein as a term of art which defines a non-enzyme protein that promotes or mediates the adsorption of the PAase of the invention onto a synthetic PA plastic or PA plastic product. Exemplary plastic binding proteins are described in Section 6.3 (below).

### 3.2.8 Single Domain KatG Enzymes

Also contemplated is the use of a KatG single domain enzyme.

Preferred single domain KatG enzymes for use according to the invention comprise, consist of, or consist essentially of, an amino acid sequence which is at least 50%, 55%, 60%, 65%, 70%, 75%, 80%, 85%, 90%, 95%, 99% or 100% identical to M1FBG9 (M1FBG9_PROMN).

M1FBG9
(SEQ ID NO: 2)
MNSLIAALLPIGVFGSCPFMDIGTPPSLKAEVISDSDFKTALEGLDIEA
LEQDLRILMTDSQTCWPADDGHYGGFMIRLAWHCAGTFRTSDQKGGCGG
AGIRFPPESDWEDNGNLDKARALLVPIKQKYGDALSWGDLISFAGTVAI
RDMGGPTNPHCFGRVDDADGNKSDIFGVTDSWQDTDCVVQGNCQEPMGA
VKVGLIYVNPEGPLNDPNDLNSGQNPDPEKSAVEIREVFGRMGMNDSET
ASLIAGGHAFGKCHGAGVMTSGFEGPWTTTPSQWTNQFLTGMLDEEWEQ
VATPSGSAVQWQTKDRTSILAGTMRLTADLALVNDDAYLALAKHWVCDQ
QKLDIAFAASWKKLVESGGGWLPVEDRRCEPESKATGQQTDPQKNFHSV
CATTTTDDSQEASTATKFCLSFLAVVLTPFSLGVHSWLA

Preferred single domain KatG enzymes are mutant forms of M1FBG9 which comprise, consist of, or consist essentially of, an amino acid sequence which is at least 50%, 55%, 60%, 65%, 70%, 75%, 80%, 85%, 90%, 95% or 99% identical to M1FBG9.

More preferred single domain KatG enzymes are mutant forms of M1FBG9 which comprise, consist of, or consist essentially of, an amino acid sequence which is at least 85%, 90%, 95% or 99% identical to M1FBG9.

Particularly preferred single domain KatG enzymes are mutant forms of M1FBG9 which comprise, consist of, or consist essentially of, an amino acid sequence which is at least 95% or 99% identical to M1FBG9.

Also contemplated are single domain KatG enzymes which are M1FBG9 orthologues.

More particularly preferred are M1FBG9 variants which comprise, consist of, or consist essentially of, an amino acid sequence which is at least 50%, 55%, 60%, 65%, 70%, 75%, 80%, 85%, 90%, 95% or 99% identical to M1FBG9 and which have one or more amino acid substitutions, deletions or insertions which:
(a) increase the polyalkenase activity; and/or
(b) increase thermostability; and/or
(c) increase psychrophilicity; and/or
(d) increase stability in relation to organic solvents, contaminants, detergents, pH and/or oxidants (including hydrogen peroxide).

Such variants can be produced by methods that are well-established in the art, including directed evolution and site-directed mutagenesis (see Zeymer and Hilvert (2018) Annual Review of Biochemistry 87:131-157; Watkins et al. Nat Commun 2017; 8:358; Watanabe and Nakajima, Methods Enzymol 2016; 580:455-470; Lichtenstein et al. Biochem Soc Trans 2012; 40:561-566; Anderson et al. Chem Sci 2014; 5:507-514; and Grayson and Anderson, Curr Opin Struct Biol 2018; 51:149-155.

### 4. KatG/EC 1.11.1.21 Polyalkenase (PAase) Substrates

### 4.1 General Considerations

The KatG/EC 1.11.1.21 polyalkenase enzymes of the invention (PAases) cleave C—C bonds in polyalkene (PA) polymer substrates, thereby enzymatically degrading the polymer into: (a) fragments; and/or (b) oligomers; and/or (c) isolated monomers or repeating units; and/or (d) monomer or repeating unit fragments; and/or (e) monomer or repeating unit derivatives.

It will be appreciated that the enzymatic degradation effected by the cleavage of C—C bonds by the PAase of the invention need not completely depolymerize the PA polymer substrate, and that the nature and extent of the degradation may be determined, inter alia, by the PAase enzyme selected, the reaction conditions and the chemical nature and/or physical form of the PA polymer substrate. Thus, the enzymatic degradation by C—C bond cleavage mediated by the PAase of the invention may merely fragment the PA polymer substrate. For example, in the case of PA copolymers which comprise long tracts (or segments) of non-PA polymers, the enzymatic degradation by C—C bond cleavage mediated by the PAase of the invention may yield fragments comprising non-PA polymer segments derived from the copolymer chain.

Alternatively, or in addition, the enzymatic degradation by C—C bond cleavage may oligomerize the PA polymer substrate. In many cases, the enzymatic degradation effected by the PAase of the invention by C—C bond cleavage yields fragments and oligomers of a PA polymer substrate. In yet other cases, the enzymatic degradation effected by the PAase of the invention by C—C bond cleavage yields: (a) fragments; and/or (b) oligomers; and/or (c) isolated monomers or repeating units; and/or (d) monomer or repeating unit fragments; and/or (e) monomer or repeating unit derivatives, of a PA polymer substrate.

In preferred embodiments, the enzymatic degradation by C—C bond cleavage effected by the PAase of the invention reduces the molecular weight of the PA polymer substrate, for example by at least 50%. More preferably, the enzymatic degradation by C—C bond cleavage effected by the PAase of the invention achieves a log reduction of at least 1, 2 or 3 of the PA polymer substrate. In cases where the polymer substrate has a relatively high molecular weight, the enzymatic degradation by C—C bond cleavage effected by the PAase of the invention may achieve correspondingly higher log reductions, for example at least 4, 5 or 6 of the PA polymer substrate.

The PA polymer substrate may be a homopolymer or a copolymer (as herein defined).

In cases where the PA polymer substrate is a linear homopolymer, the C—C bonds cleaved by the PAase of the invention are located in the backbone. In cases where the PA polymer substrate is a branched homopolymer, the C—C bonds cleaved by the PAase of the invention may be located in the backbone, in the branches, or in both the backbone and the branches of the polymer.

In cases where the PA polymer substrate is a copolymer comprising two or more different polymerized alkene monomers, the C—C bonds cleaved by the PAase of the invention may be located between any of the different polymerized alkene monomers, or only between polymerized monomers of the same species. These C—C bonds may be located in the backbone and/or in side branches (if present).

In cases where the PA polymer substrate is a copolymer comprising polymerized alkene and non-alkene monomers, the C—C bonds cleaved by the PAase of the invention may be located only between polymerized alkene monomers. In the latter case, the C—C bonds may be located in the backbone (for example in the case of certain block copolymers), or in side branches (for example in the case of certain graft copolymers).

In cases where the PA polymer substrate is an alternate copolymer comprising polymerized alkene and non-alkene monomers, the C—C bonds cleaved by the PAase of the invention are located within repeating polymerized alkene units. In such embodiments, the enzymatic degradation effected by the PAase of the invention by C—C bond cleavage may yield polymer fragments or oligomers comprising both polymerized alkene and non-alkene monomers.

In cases where the PA polymer substrate is a random copolymer comprising alkene and non-alkene monomers, the C—C bonds cleaved by the PAase of the invention are located within repeating polymerized alkene monomers. In such embodiments, the enzymatic degradation effected by the PAase of the invention by C—C bond cleavage may yield polymer fragments or oligomers comprising both polymerized alkene and non-alkene monomers as well as fragments containing only polymerized non-alkene monomers (for example, corresponding to non-PA polymer segments derived from the copolymer chain).

In cases where the PA polymer substrate is a block copolymer comprising alkene and non-alkene blocks, the C—C bonds cleaved by the PAase of the invention are located within an alkene block (and may be located only within said alkene block). In such embodiments, the enzymatic degradation effected by the PAase of the invention by C—C bond cleavage may yield polymer fragments or oligomers corresponding to the non-alkene blocks (where the blocks may, for example, correspond to non-PA polymer segments derived from the copolymer chain).

In cases where the PA polymer substrate is a graft copolymer comprising an alkene backbone and non-alkene side chains, the C—C bonds cleaved by the PAase of the invention are located within the alkene backbone (and may be located only within said alkene backbone). In such embodiments, the enzymatic degradation effected by the PAase of the invention by C—C bond cleavage may yield polymer fragments or oligomers corresponding to the non-alkene side chains.

In cases where the PA polymer substrate is a graft copolymer comprising a non-alkene backbone and alkene side chains, the C—C bonds cleaved by the PAase of the invention are located within the alkene side chains (and may be located only within said alkene side chains). In such embodiments, the enzymatic degradation effected by the PAase of the invention by C—C bond cleavage may yield polymer fragments corresponding to the non-alkene backbone.

### 4.2 Preferred PA Polymer Substrates

Preferred PA polymer substrates comprise polymer segments having a C—C backbone and include the various types of polyethylene (PE) and structurally related polyalkene polymers (which may be considered as substituted polyethylenes).

These preferred PA polymer substrates therefore include various types of polyethylene (PE), polypropylene (PP), polystyrene (PS), polyvinyl chloride (PVC) and chlorinated polyethylene (CPE), all of which comprise polymer tracts or segments having a C—C backbone, and which may be represented schematically as shown below:

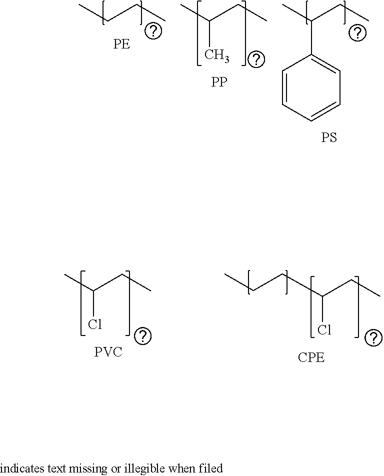

### 4.2.1 Polyethylene (PE)

In preferred embodiments, the PA polymer substrate is a polyethylene (PE) polymer. Thus, preferred PAases of the invention have PE degrading activity (and so may be referred to herein as PEases of the invention). PE is the most widely produced synthetic plastic polymer—116 million tons were produced in 2016 (Danso et al., op. cit.).

Examples of the preferred PE substrates include high-density PE (HDPE), medium-density PE (MDPE), low-density PE (LDPE), linear low-density PE (LLDPE), ultra-low-molecular-weight polyethylene (ULMWPE), high-molecular-weight polyethylene (HMWPE), ultra-high molecular weight polyethylene (UHMWPE), high-density cross-linked polyethylene (HDXLPE), cross-linked polyethylene (PEX or XLPE), linear polyethylene (LPE) and very-low-density polyethylene (VLDPE).

HDPE typically has a molecular weight of in the range 20,000-60,000 Da (700 to 1,800 monomer units) and a density of at least 0.941 g/cm³. It comprises a C—C backbone which has a low degree of branching, which may be represented thus:

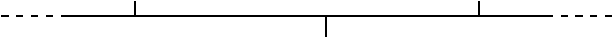

The mostly linear molecules pack together well, so intermolecular forces are stronger than in highly branched polymers. HDPE is produced by chromium/silica catalysts, Ziegler-Natta catalysts or metallocene catalysts and the small amount of branching can be controlled since these catalysts promote the formation of free radicals at the ends of the growing polyethylene backbone chains. It has high tensile strength and is used in products and packaging such as milk jugs, detergent bottles, butter tubs, garbage containers, and water pipes. One-third of all toys are manufactured from HDPE.

MDPE has a density range of 0.926-0.940 g/cm³. It is typically used in gas pipes and fittings, sacks, shrink film, packaging film, carrier bags, and screw closures.

LDPE has a density range of 0.910-0.940 g/cm³. It has a high degree of short- and long-chain branching, which means that the chains do not pack well into the crystal structure. Its branched C—C backbone may be represented thus:

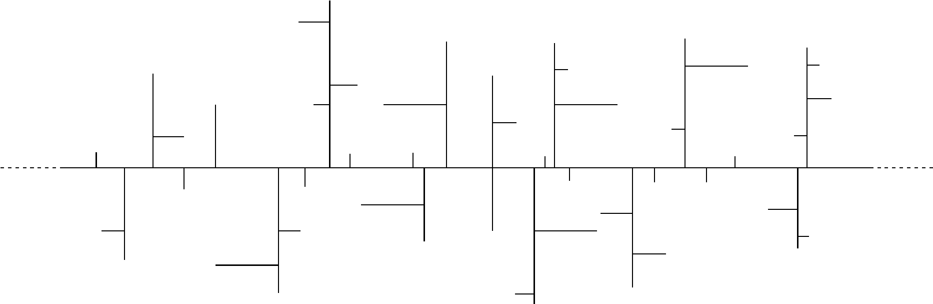

LDPE therefore exhibits a relatively low tensile strength and relatively high ductility. LDPE is created by free-radical polymerization. The high degree of branching with long chains gives molten LDPE unique and desirable flow properties. It is used for both rigid containers and plastic film applications such as plastic bags and film wrap.

LLDPE is defined by a density range of 0.915-0.925 g/cm³. It is a substantially linear copolymer with significant numbers of short branches, commonly made by copolymerization of ethylene with short-chain alpha-olefins (for example, 1-butene, 1-hexene, and 1-octene). Its branched C—C backbone may be represented thus:

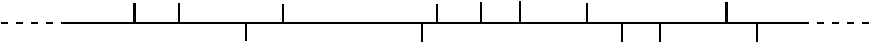

LLDPE has higher tensile strength than LDPE, and it exhibits higher impact and puncture resistance than LDPE. Lower-thickness (gauge) films can be blown, compared with LDPE, with better environmental stress cracking resistance, but they are not as easy to process. It is used in packaging, particularly film for bags and sheets. Lower thickness may be used compared to LDPE. It is used for cable coverings, toys, lids, buckets, containers, and pipe. While other applications are available, LLDPE is used predominantly in film applications due to its toughness, flexibility, and relative transparency.

Linear PE is normally produced with molecular weights in the range of 200,000 Da to 500,000 Da. Polyethylene with molecular weights of 3-8×10⁶ Da is referred to as ultra-high molecular weight polyethylene, or UHMWPE, and typically has 100,000 to 250,000 monomer units per molecule.

Cross-linked PE (PEX or XLPE) is a medium- to high-density polyethylene containing cross-links (which yield a thermoset rather than thermoplastic). Temperature resistance is higher than un-crosslinked Pes, and chemical resistance is enhanced.

Chlorinated polyethylene CPE or PE-C) is an inexpensive variant of PE having a chlorine content from 34 to 44%. It is used in blends with polyvinyl chloride (PVC) because the soft, rubbery chlorinated polyethylene is embedded in the PVC matrix, thereby increasing the impact resistance. Chlorination also increases weather resistance. Like PE, CPE can be crosslinked to form an elastomer which is used in cable and rubber industry. CPE is often added to other polyalkene polymers to reduce flammability. Preferred PAases of the invention have CPE degrading activity (and so may be referred to herein as CPEases of the invention).

PE is also produced in a number of copolymer forms. Copolymers with unsaturated carboxylic acids (such as acrylic acid to form ethylene acrylic acid, EAA) find application as adhesives. Copolymers with unsaturated esters may have the alcohol moiety located on the polymer backbone. An example is ethylene vinyl alcohol (EVOH) as shown below:

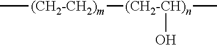

As shown above, EVOH contains a C—C backbone polymer segment of m repeating units. EVOH is highly transparent, weather resistant, oil and solvent resistant, flexible, moldable and is commonly used as an oxygen barrier in food packaging.

Other copolymers with unsaturated esters have the acid moiety located on the polymer backbone. For example, ethylene-ethyl acrylate (EEA) copolymer has the following structure:

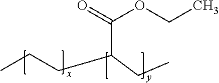

The related ethylene-butyl acrylate (EBA) has the structure:

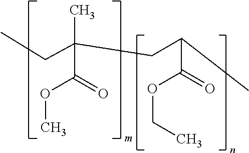

### 4.2.2 Polypropylene (PP)

In other preferred embodiments, the PA polymer substrate is a polypropylene (PP) polymer. Thus, preferred PAases of the invention have PP degrading activity (and so may be referred to herein as PPases of the invention). PP is the second-most widely produced synthetic plastic polymer (after polyethylene)—68 million tons were produced in 2016 (Danso et al., op. cit.). PP is produced via chain-growth polymerization from the monomer propylene. It is a thermoplastic polymer used in a wide variety of applications. Its physical properties are similar to those of PE, but it is slightly harder and more heat resistant. It is often used in packaging and labelling.

Polypropylene is categorized as atactic (PP-at), syndiotactic (PP-st) and isotactic (PP-it), according to the alignment of the methyl groups. These may be aligned randomly (PP-at), alternately (PP-st) or evenly (PP-it). This has an impact on crystallinity (amorphous or semi-crystalline) and thermal properties (including the glass transition and melting points).

PP homopolymer is the most common PP grade. It contains only propylene monomer in a semi-crystalline solid form. Applications include packaging, textiles, healthcare, pipes, automotive and electrical.

PP is also produced as a copolymer. PP copolymer includes random copolymer and block copolymer, both produced by polymerizing propene and ethene monomers. Random PP copolymer is produced by polymerizing ethene and propene in which ethane monomers typically constitute up to 6% by mass and are incorporated randomly in the polypropylene chains. These polymers are flexible and optically clear. Block PP copolymer contains a higher ethene content (typically 5-15%) and is a tougher and less brittle thermoplastic than the random co-polymer.

### 4.2.3 Polyvinylchloride (PVC)

In other preferred embodiments, the PA polymer substrate is a polyvinyl chloride (PVC) polymer. Thus, preferred PAases of the invention have PVC degrading activity (and so may be referred to herein as PVCases of the invention). Polyvinyl chloride (PVC) is the third-most widely produced synthetic plastic polymer (after polyethylene and polypropylene)—38 million tons were produced in 2016 (Danso et al., op. cit.).

PVC is produced in two forms: rigid (sometimes abbreviated as RPVC) and flexible. The rigid form of PVC is used in construction for pipe and in profile applications such as doors and windows. It is also used in making bottles, non-food packaging, food-covering sheets and cards (such as bank or membership cards). It can be made softer and more flexible by the addition of plasticizers, the most widely used being phthalates. In this form, it is also used in plumbing, electrical cable insulation, imitation leather, flooring, signage, phonograph records, inflatable products, and in many other applications where it replaces rubber.

Polyvinylidene chloride (PVDC) may be considered as a variant of PVC, having the structure:

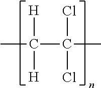

PVC is also produced as a copolymer, for example with vinyl chloride and vinyl acetate monomers. Similarly, PVDC copolymers are produced. For example, copolymers of about 87% vinylidene chloride and 13% vinyl chloride have been marketed as Saran™ and used in extruded form as a food wrap.

### 4.2.4 Polystyrene (PS)

In other preferred embodiments, the PA polymer substrate is a polystyrene (PS) polymer. Thus, preferred PAases of the invention have PS degrading activity (and so may be referred to herein as PSases of the invention). PS is the sixth-most widely produced synthetic plastic polymer (after PE, PP, PVC, PET and PUR)—14 million tons were produced in 2016 (Danso et al., op. cit.). PS is produced by the polymerization of styrene monomers. Each PS molecule typically consists of a few thousand monomers, yielding a molecular weight in the range 100,000-400,000 Da.

PS is manufactured in solid and foamed forms. General-purpose polystyrene is clear, hard and brittle. It is an inexpensive resin per unit weight. It is a rather poor barrier to oxygen and water vapour and has a relatively low melting point. Uses include protective packaging (such as CD and DVD cases), containers, lids, bottles, trays, tumblers and disposable cutlery.

As a thermoplastic polymer, PS is a glassy solid at room temperature and flows above about 100° C. (its glass transition temperature). This is exploited for extrusion (as in Styrofoam), and also for moulding and vacuum forming, since it can be cast into moulds with fine detail.

Like PP, PS is categorized as atactic (PS-at), syndiotactic (PS-st) and isotactic (PS-it), according to the alignment of the phenyl groups (i.e. randomly (PS-at), alternately (PS-st) or evenly (PS-it). Standard polystyrene is atactic. Isotactic polystyrene is not currently produced commercially.

Like PP, PS is also produced in copolymer forms. While homopolymeric PS has excellent transparency, surface quality and stiffness, its versatility is extended by copolymerization and other modifications. Several copolymers are used based on styrene. Styrene-butadiene copolymers (in both random and block copolymer forms) overcome problems associated with the brittleness of homopolymeric polystyrene, while copolymers of styrene and acrylonitrile (SAN) are more resistant to thermal stress, heat and chemicals (while retaining transparency). PS copolymers include styrene maleic anhydride (SMA—a copolymer with maleic anhydride), styrene-acrylonitrile resin (SAN) and acrylonitrile-butadiene-styrene (ABS), which is described in more detail below.

### 4.2.5 Acrylonitrile-butadiene-styrene (ABS)

In other preferred embodiments, the PA polymer substrate is an acrylonitrile-butadiene-styrene (ABS) polymer. Thus, preferred PAases of the invention have ABS degrading activity (and so may be referred to herein as ABSases of the invention).

The terpolymer ABS is made by polymerizing styrene and acrylonitrile in the presence of polybutadiene. The proportions can vary from 15 to 35% acrylonitrile, 5 to 30% butadiene and 40 to 60% styrene. The result is a long chain of polybutadiene criss-crossed with shorter chains of poly(styrene-co-acrylonitrile) as shown schematically below:

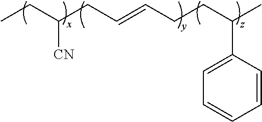

### 4.2.5 Polybutadiene (BR)

Polybutadiene (also known as butadiene rubber, BR) which may be represented schematically as shown below:

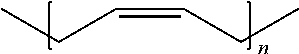

BR is a synthetic rubber formed from the polymerization of the monomer 1,3-butadiene. Polybutadiene has a high resistance to wear and is used in the manufacture of vehicle tires (which account for about 70% of production). It is also widely used as an additive to improve the impact resistance of other plastics (including the PS and ABS polymers described above). It is also used to manufacture golf balls and to encapsulate electronic assemblies.

### 4.2.7 Polyisoprene (IR)

In other preferred embodiments, the PA polymer substrate is a polyisoprene (IR) polymer. Thus, preferred PAases of the invention have IR degrading activity (and so may be referred to herein as IRases of the invention).

Hevea rubber and Gutta-percha are non-synthetic rubbers (natural rubber, NR) commercially exploited since the beginning of the 20^{th} century. Hevea rubber is commercially extracted from the sap of the Hevea brasiliensis tree and Gutta-percha from trees of the genus Palaquium. Both natural rubbers consist of cis-1,4-polyisoprene with an average molecular mass of about 10⁶ Da:

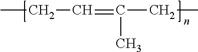

In natural form, natural rubbers are therefore fully biodegradable due to the presence of double bonds in the polymer backbone that are prone to thermal and oxidative degradation (which leads to chain scission).

The synthetic equivalent is polyisoprene (IR), a collective name for polymers that are produced by polymerization of isoprene by polymerization of 2-methyl-1,3-butadiene with Ziegler-Natta catalyst. The various isomeric forms of the repeating units may be represented schematically as shown below:

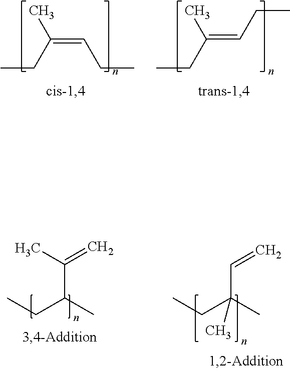

Synthetic isoprene is a blend of cis-1,4, trans-1,4 and 3,4 polymer, with cis-1,4 typically present in the range of 90% to 98%. An increase in cis-1,4 usually lowers the glass transition temperature, increases the crystallinity, and improves the mechanical strength.

Both NR and IR are usually converted into rubber products by the process of vulcanization that leads to cross-links between the polymer chains either by heating in the presence of elemental sulfur (e.g., during the manufacture of tires that typically also contain BR) or by irradiation and peroxidation, respectively (as in the case of NR latex gloves). IR can also be produced using biological process, for example from isoprene monomers produced from glucose using metabolic pathway engineering (Whited et al. (2010) Industrial Biotechnology 6: 152-163).

### 4.3 Synthetic PA Plastic Substrates

As explained above, the PAases of the invention cleave C—C bonds in polyalkene (PA) polymer substrates, thereby enzymatically degrading the polymer. The various PA polymer substrates described in Sections 4.1 and 4.2 may serve as PAase substrates when in the form of a synthetic PA plastic (as herein defined). Thus, preferred PAases of the invention have synthetic PA plastic degrading activity, and so find application in the degradation of synthetic PA plastics.

Synthetic PA plastics as herein defined have a molecular weight of at least 5000 Da, and more typically have a molecular weight of at least 20,000 Da. In this regard, it is noted that synthetic PA polymers comprising a C—C backbone/backbone segment include synthetic PE waxes which may have molecular weights in the range of 5000-20,000 Da. It will therefore be appreciated that natural polymers (such as certain waxes), which may comprise polyalkene tracts, segments, domains or substructures, have much lower molecular weights, as do synthetic paraffin waxes and oils. The foregoing polymers are therefore not synthetic PA plastics as herein defined.

Synthetic PA plastics typically comprise crystalline, semi-crystalline and/or amorphous domains, in most cases comprising both crystalline (and/or or semi-crystalline) and amorphous domains. The degree of crystallinity is a determinant of certain functionally important structural properties. Synthetic plastic PA polymer substrates are typically solid thermoplastic materials at room temperature. However, some chemically modified and/or copolymer synthetic PA plastics (e.g. PEX) may be thermoset plastics.

### 4.4 Plastic Products as Synthetic PA Plastic Substrates

As explained above, the PAases of the invention cleave C—C bonds in polyalkene (PA) polymer substrates, thereby enzymatically degrading the polymer. Moreover, the various PA polymer substrates described in Sections 4.1 and 4.2 may serve as PAase substrates when in the form of synthetic PA plastics (as explained in Section 4.3).

As described below, the synthetic PA plastics described in Section 4.3 may serve as PAase substrates when in the form of any of a wide variety of PA plastic products. Thus, preferred PAases of the invention have PA plastic product degrading activity, and so find application in the degradation of PA plastic products.

As used herein, the term PA plastic product defines a plastic article comprising one or more synthetic PA plastics which has been formed into an article. The plastic article may be a moulded plastic article. It may be characterized by physical conformations selected from sheets, films, tubes, pipes, straws, rods, ropes, strings, lines, nets, reticulated sheets, three dimensional regular shapes, blocks, sheaths, fibres, membranes, woven and non-woven textiles, bags, containers, bottles, capsules, packaging, discs, microspheres, (electro)mechanical components, clothing, single-use coffee cups, single-use coffee capsules, building components, Lego® bricks, coatings, panels and moulded and extruded profiles.

The PA plastic product may comprise a single class of synthetic PA plastic (for example, comprising a synthetic PE plastic alone), or may comprise a combination of two or more different synthetic PA plastics (for example, a combination of synthetic PE and PP plastics). Alternatively, the PA plastic product may comprise one or more synthetic PA plastics (for example PE, PP and/or PVC) in combination with one or more synthetic non-PA plastics (for example PET and/or PUR and/or polyamides).

When present in combination with other plastics, the synthetic PA plastic may form part of a mixture with other synthetic non-PA plastics. Alternatively, or in addition, the synthetic PA plastic may be present as part of a composite plastic product (for example, as a coating, layer or inclusion, for example following co-melting) or as a mechanically, physically and/or chemically attached component part thereof. For example the PA plastic product may comprise successive layers of different plastic materials.

The PA plastic product substrates may further comprise various additives, such as acid scavengers, prodegradants, antioxidants, antistatic agents, flame-retardants, light stabilizers, thermal stabilizers, lubricants, non-slip compounds, plasticizers, pigments, dyes and mineral or organic fillers (including metals, such as cadmium, chromium, lead, mercury, cobalt and zinc). Common additives in PS polymers include: 2-(2′-hydroxy-5′-methylphenyl)benzotriazole (Tinuvin P), Acrawax, Alicyclic bromine, bis (2, 2, 6, 6-tetramethyl-4-piperidyl)sebacate (Tinuvin 770), decabromodiphenyl ethane (S-8010), decabromodiphenyl oxide, dibromoethyldibromocyclohexane (Saytex BCL-462), ethylene bistetrabromonorbornane dicarboximide (Saytex BT-93), hexabromocyclododecane, mineral oil, octadecyl 3,5-di-tert-butyl-4-hydroxycinnamate (Irganox 1076), pentabromochlorocyclohexane, stearic acid, tetrabromobisphenol A,Tris Nonyl Phenyl Phosphite (Wytox) and zinc stearate.

It will be appreciated that the PA plastic product degrading activity of the PAases of the invention need not completely degrade the PA plastic product, and that the nature and extent of the degradation may be determined, inter alia, by the PAase enzyme selected, the reaction conditions and the chemical nature and/or physical form of the PA plastic product substrate.

The PA plastic product substrates may comprise, or be derived from, domestic, commercial or industrial mixed or sorted waste streams or collected plastic wastes. For example, the PA plastic products for use as PAase substrates may be selected from plastic bottles, plastic bags, plastic packaging and textile waste.

Thus, in certain embodiments, the PA plastic product may comprise a synthetic PA plastic and one or more additional plastics. The additional plastics may, for example, be selected from ethylene vinyl alcohol (EVOH), polyethylene furanoate (PEF), polyurethane (PUR), acrylonitrile butadiene styrene (ABS), poly(oxide phenylene) (PPO), polycarbonate (PC), copolymer of phosphono and carboxylic acid (PCA), polyacrylate, polymethacrylate methyle (PMMA), polyoxymethylene (POM), styrene acrylonitrile (SAN), polyester polymer alloy (PEPA), polyethylene naphthalate (PEN), styrene-butadiene (SB), and blends/mixtures of the foregoing plastics.

In preferred embodiments, the additional plastics are selected from polyesters and/or polyamides. Preferred polyesters are polyethylene terephthalate (PET), polytrimethylene terephthalate (PTT), polybutylene terephthalate (PBT), polyethylene isosorbide terephthalate (PEIT), polylactic acid (PLA), poly(L-lactic acid) (PLLA), poly(D-lactic acid) (PDLA), poly(D,L lactic acid) (PDLLA), PLA stereocomplex (scPLA), polyhydroxy alkanoate (PHA), poly(3-hydroxybutyrate) (P(3HB)/PHB), poly(3-hydroxyvalerate) (P(3HV)/PHV), poly(3-hydroxyhexanoate) (P(3HHx)), poly(3-hydroxyoctanoate) (P(3HO)), poly(3-hydroxydecanoate) (P(3HD)), poly(3-hydroxybutyrate-co-3-hydroxyvalerate) (P(3HB-co-3HV)/PHBV), poly(3-hydroxybutyrate-co-3-hydroxyhexanoate) (P(3HB-co-3HHx)/(PHBHHx)), poly(3-hydroxybutyrate-co-5-hydroxyvalerate) (PHB5HV), poly(3-hydroxybutyrate-co-3-hydroxypropionate) (PH B3H P), pol yhydroxybutyrate-co-hydroxyoctonoate (PH BO), polyhydroxybutyrate-co-hydroxyoctadecanoate (PHBOd), poly(3-hydroxybutyrate-co-3-hydroxyvalerate-co-4-hydroxybutyrate) (P(3HB-co-3HV-co-4HB)), polybutylene succinate (PBS), polybutylene succinate adipate (PBSA), polybutylene adipate terephthalate (PBAT), polyethylene furanoate (PEP), polycaprolactone (PCL), polyethylene adipate) (PEA) and blends/mixtures of the foregoing plastics.

Preferred polyamides (nylons) are polyamide-6 or poly(β-caprolactam) or polycaproamide (PA6), polyamide-6,6 or poly(hexamethylene adipamide) (PA6,6), poly(11-aminoundecanoamide) (PA11), polydodecanolactam (PA12), poly(tetramethylene adipamide) (PA4,6), poly(pentamethylene sebacamide) (PA5,10), poly(hexamethylene azelaamide) (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T), polyhexamethylene adipamide/polyhexamethyleneisophtalamide copolymer (PA66/6I) and blends/mixtures of the foregoing plastics.

### 5. Processed Plastic Products for Reaction Mix Compositions of the Invention

### 5.1 General Considerations

As explained in Sections 4.1-4.3 (above), the PAases of the invention may have PA plastic product degrading activity, and so find application in the degradation of PA plastic products. As further explained in Section 4.4 (above), any of a wide range of PA plastic products may serve as substrates for the PAases of the invention.

In such applications, the PAase and PA plastic product may form part of a reaction mix composition for use in the reactors of the invention. Such reaction mixes comprise the PAase of the invention in combination with the PA plastic product to be degraded.

In preferred embodiments of the reaction mix, the PA plastic product is processed before, during or after incorporation into the reaction mix (and/or contact with the PAase). For example, the PA plastic product may be processed before (i.e. by some form of pre-treatment), in the course of PAase-mediated enzymatic degradation, or in a later, downstream process.

In preferred embodiments, the processing comprises pre-treatment of the PA plastic product (i.e. prior to contact with the PAase of the invention).

Thus, the invention contemplates the use of the PAase enzyme of the invention together with one or more abiotic and/or biotic processing steps (for example, enzymatic degradation and/or incubation with one or more microorganisms and/or insects/insect larvae) simultaneously or sequentially to degrade PA plastic products, particularly in circumstances where the PA plastic product:
(a) forms part of domestic, commercial or industrial mixed or sorted waste streams or collected plastic wastes; and/or
(b) is present in combination with other plastics, either as part of a mixture with other synthetic non-PA plastics; and/or
(c) is present as part of a composite plastic product (for example, as a coating, layer, inclusion or dispersed phase, for example following co-melting) or as a mechanically, physically and/or chemically attached component part thereof (for example where the PA plastic product comprises layers of different plastic materials (e.g. laminates) and/or inter-operable components and/or mechanically-coupled components and/or spatially separated arrays).

Such processing may promote or accelerate the PAase-mediated enzymatic degradation of the PA plastic product (and/or the enzymatic degradation mediated by any adjunctive or complementary plasticase enzymes that may also be present—see Section 5.3.1).

Thus, such processing may:
(a) increase the rate of enzymatic PAase degradation (and/or of any of the adjunctive or complementary plasticase enzymes described in Section 5.3.1, if present); and/or
(b) increase the extent of enzymatic PAase degradation (and/or of any of the adjunctive or complementary plasticase enzymes described in Section 5.3.1, if present); and/or
(c) permit enzymatic PAase degradation of PA plastic products which would not otherwise serve as PAase substrates (for example, in cases where the PA plastic product is coated with a plastic which is not a PAase substrate). Analogous considerations apply with regard to any of the adjunctive or complementary plasticase enzymes described in Section 5.3.1, if present.

In some embodiments, the processing described above physically changes the structure of the PA plastic product, preferably increasing the surface area available for contact with the PAase enzyme and/or exposing crystalline or semicrystalline domains within the PA plastic product to the PAase enzyme. Thus, grinding is a preferred abiotic processing step.

In other embodiments, the processing described above chemically changes the PA plastic product, preferably increasing its susceptibility to PAase mediated enzymatic degradation.

In yet other embodiments, the processing described above does not change the PA plastic product, but rather changes (physically and/or chemically) other materials that are associated with the PA plastic product. Such materials include other plastics (including those described in Section 4.4, above), non-plastic pollutants (including those described in Section 5.3.2, below) and lignocellulosic biomass (also described in Section 5.3.2, below), the latter being of particular importance in cases where the PAase substrate is present as part of a domestic, commercial or industrial mixed waste stream containing plastics mixed with lignocellulosic materials (such as domestic waste, industrial waste, garden waste, kitchen waste, agricultural waste, sawdust, wood chips, cardboard, cardboard pulp, paper, paper pulp and plastic and non-plastic packaging).

### 5.2 Abiotic Processing

In preferred embodiments, the processing of the PA plastic product is abiotic. Exemplary abiotic treatments include treatments selected from mechanical, physical and chemical treatments, or a combination of two or more thereof.

Abiotic processing is preferably conducted before, or during, contact with the PAase of the invention, i.e. as a pre-treatment or as a co-processing step. Abiotic processing is most preferably conducted as a pre-treatment processing step.

Abiotic processing may, for example, comprise mechanical processing of the PA plastic product by a treatment selected from: washing, centrifugation, cleaning, collision, sorting, grinding, homogenization (for example, high-pressure homogenization), shredding, cutting, impacting, crushing, shearing, shredding (e.g. disc screen shredding), rotary drum tumbling, fractionation, sonication, melting, extrusion, spinning, liquefaction, maceration, micronization, pelletization, screw pressing, piston pressing and granulation, and a combination of two or more of the foregoing.

Grinding is particularly preferred, optionally together with one or more of the other abiotic processing steps listed above.

Washing, cleaning and/or sorting are particular preferred as pre-treatments in cases where the PA plastic product forms part of domestic, commercial or industrial mixed or sorted waste streams or collected plastic wastes.

Alternatively, or in addition, the PA plastic product may be physically processed by a treatment selected from: irradiation, for example drying, UV irradiation, amorphization, agglomeration, heating (e.g. by microwaves), cooling, freezing, dessication, and a combination of two or more of the foregoing.

In preferred embodiments, the physical treatment of the PA plastic product comprises amorphization. Suitable amorphization treatments are described in WO2017198786. They include heating, for example in an extruder. Such an amorphizing step may comprise heating to a temperature which is above: (a) the crystallization temperature (Tc) of the PA polymer in the plastic product; and/or (b) the melting temperature (Tm) of the PA polymer in the plastic product.

Alternatively, or in addition, the PA plastic product may be chemically processed by oxidation.

Alternatively, or in addition, the PA plastic product may be processed by contact with a surfactant or plastic binding agent. Plastic binding agents mediate adsorption of the PAase to the plastic substrate, while surfactants may promote access of the PAase to the PA plastic product, for example by facilitating contact of the PAase with hydrophobic surfaces of the plastic product. Preferred surfactants include non-ionic surfactants. Other preferred surfactants include Tween 80, Tween 20 and CHAPSO (which have been shown to promote the degradation of PE by a partially purified MnP (as described by liyoshi et al. (1998) op. cit. and Ehara et al. (2000) op. cit.).

Alternatively, or in addition, the PA plastic product may be processed by treatment with a plastic binding protein (PBP). PBPs are non-enzyme proteins that promote or mediate the adsorption of the PAase of the invention onto a synthetic PA plastic or PA plastic product, and are described in detail in Section 6.3, below).

### 5.3 Biotic Processing

As an alternative, or preferably in addition, to the abiotic processing described in Section 5.2, the processing of the PA plastic product comprises a biotic process. In preferred embodiments, one or more of the biotic processes described below are used in addition to grinding.

Biotic processing includes contacting the PA plastic product with: (a) one or more enzymes other than the PAase of the invention; and/or (b) one or more microorganisms and/or (c) one or more insects/insect larvae.

### 5.3.1 Adjunctive Plasticase Treatment

It will be appreciated that the PA plastic product may not be completely degraded by the enzymatic activity of the PAases of the invention, and that the nature and extent of the degradation may be determined, inter alia, by the PAase enzyme selected, the reaction conditions and the chemical nature and/or physical form of the PA plastic product substrate.

Thus, processing of the PA plastic product with one or more adjunctive plasticase enzymes other than the PAase of the invention may: (a) increase the rate of enzymatic PAase degradation (for example, by accelerating the rate at which substrate is made available to the PAase; and/or (b) increase the extent of enzymatic PAase degradation (for example, by liberating a fraction of the PA plastic product which would otherwise be sequestered from the PAase of the invention); and/or (c) permit enzymatic PAase degradation of PA plastic products which would not otherwise serve as PAase substrates (for example, in cases where the PA plastic product is coated with a plastic which is not a PAase substrate but which is degraded by processing with the other enzyme(s)).

As used herein, the term adjunctive plasticase defines an enzyme which enzymatically degrades one or more synthetic plastic polymers, but which is not a PAase (as herein defined). Preferred adjunctive plasticase enzymes include augmentary plasticases.

### Augmentary Plasticases

As used herein, the term augmentary plasticase defines an enzyme which exhibits (at least some) degradative activity against a PA polymer, but which is not a PAase (as herein defined).

Preferred augmentary plasticase enzymes include: (a) laccase (EC 1.10.3.2.); (b) manganese peroxidase (MnP, EC 1.11.1.13); (c) lignin peroxidase (LiP, EC 1.11.1.14); and (d) combinations of two or more of the foregoing.

### Complementary Plasticases

Also preferred as adjunctive plasticases are complementary plasticases, particularly in circumstances where the PA plastic product: (a) forms part of domestic, commercial or industrial mixed or sorted waste streams or collected plastic wastes; and/or (b) is present in combination with other plastics, either as part of a mixture with other synthetic non-PA plastics and/or is present as part of a composite plastic product (for example, as a coating, layer or inclusion, for example following co-melting) or as a mechanically, physically and/or chemically attached component part thereof (for example where the PA plastic product may comprise successive layers of different plastic materials).

As used herein, the term complementary plasticase defines an enzyme which exhibits (at least some) degradative activity against a non-PA polymer, and which is not a PAase (as herein defined).

Complementary plasticases may therefore degrade one or more of the additional plastic substrates listed in Section 4.4 (above), and so include enzymes with degrade plastics selected from ethylene vinyl alcohol (EVOH), polyethylene furanoate (PEF), polyurethane (PUR), acrylonitrile butadiene styrene (ABS), poly(oxide phenylene) (PPO), polycarbonate (PC), copolymer of phosphono and carboxylic acid (PCA), polyacrylate, polymethacrylate methyle (PMMA), polyoxymethylene (POM), styrene acrylonitrile (SAN), polyester polymer alloy (PEPA), polyethylene naphthalate (PEN) and styrene-butadiene (SB).

The complementary plasticase may therefore degrade polyesters and/or polyamides.

Polyesters include polyethylene terephthalate (PET), polytrimethylene terephthalate (PTT), polybutylene terephthalate (PBT), polyethylene isosorbide terephthalate (PEIT), polylactic acid (PLA), poly(L-lactic acid) (PLLA), poly(D-lactic acid) (PDLA), poly(D,L lactic acid) (PDLLA), PLA stereocomplex (scPLA), polyhydroxy alkanoate (PHA), poly(3-hydroxybutyrate) (P(3HB)/PHB), poly(3-hydroxyvalerate) (P(3HV)/PHV), poly(3-hydroxyhexanoate) (P(3HHx)), poly(3-hydroxyoctanoate) (P(3HO)), poly(3-hydroxydecanoate) (P(3HD)), poly(3-hydroxybutyrate-co-3-hydroxyvalerate) (P(3HB-co-3HV)/PHBV), poly(3-hydroxybutyrate-co-3-hydroxyhexanoate) (P(3HB-co-3HHx)/(PHBHHx)), poly(3-hydroxybutyrate-co-5-hydroxyvalerate) (PHB5HV), poly(3-hydroxybutyrate-co-3-hydroxypropionate) (PHB3HP), pol yhydroxybutyrate-co-hydroxyoctonoate (PHBO), polyhydroxybutyrate-co-hydroxyoctadecanoate (PHBOd), poly(3-hydroxybutyrate-co-3-hydroxyvalerate-co-4-hydroxybutyrate) (P(3HB-co-3HV-co-4HB)), polybutylene succinate (PBS), polybutylene succinate adipate (PBSA), polybutylene adipate terephthalate (PBAT), polyethylene furanoate (PEF), polycaprolactone (PCL), and polyethylene adipate) (PEA).

Polyamides (nylons) include polyamide-6 or poly(β-caprolactam) or polycaproamide (PA6), polyamide-6,6 or poly(hexamethylene adipamide) (PA6,6), poly(11-aminoundecanoamide) (PA11), polydodecanolactam (PA12), poly(tetramethylene adipamide) (PA4,6), poly(pentamethylene sebacamide) (PA5,10), poly(hexamethylene azelaamide) (PA6,9), poly(hexamethylene sebacamide) (PA6,10), poly(hexamethylene dodecanoamide) (PA6,12), poly(m-xylylene adipamide) (PAMXD6), polyhexamethylene adipamide/polyhexamethyleneterephtalamide copolymer (PA66/6T) and polyhexamethylene adipamide/polyhexamethyleneisophtalamide copolymer (PA66/6I).

Exemplary complementary plasticases include: (a) laccases (EC 1.10.3.2.); (b) manganese peroxidases (MnP, EC 1.11.1.13); (c) lignin peroxidases (LiP, EC 1.11.1.14); (d) versatile peroxidases; (e) proteases; (f) lipases; (g) a carboxylesterases; (h) an esterases; and (i) combinations of two or more of the foregoing. Particularly preferred complementary plasticases include cutinases and/or PETases.

Exemplary complementary plasticases include oxidases, preferably selected from the group consisting of a laccase, lipoxygenase, peroxidase, haloperoxidase, mono-oxygenase, dioxygenase, peroxygenase and hydroxylase.

Suitable lignin and manganese peroxidases include those from Streptomyces sp. and Phanerochaete chrysosporium.

Suitable laccases include those from Rhodococcus ruber DSM 45332 and the commercial laccase from Trametes versicolor.

Particularly preferred complementary plasticases include cutinases and/or PETases, for example selected from cutinase (EC 3.1.1.74), lipase (EC 3.1.1.3), esterase, carboxylesterase (EC 3.1.1.1), p-nitrobenzylesterase, serine protease (EC 3.4.21.64), protease, amidase, aryl-acylamidase (EC 3.5.1.13), oligomer hydrolase (such as 6-aminohexanoate cyclic dimer hydrolase (EC 3.5.2.12)), urethanase, 6-aminohexanoate dimer hydrolase (EC 3.5.1.46), 6-aminohexanoate-oligomer hydrolase (EC 3.5.1.117), peroxidase and laccase (EC 1.10.3.2).

Suitable serine proteases include proteinase K from Tritirachium album and PLA depolymerase from Amycolatopsis sp. A serine protease from Actinomadura sp. finds particular application in the degradation of plastic products comprising polylactic acid.

Suitable lipases include those from Candida antarctica, Cryptococcus sp., Burkholderia cepacia and Aspergillus niger), and find particular application in the degradation of plastic products comprising PET or PTT.

Suitable esterases include those from Thermobifida halotolerans, Pseudomonas sp. and Chaetomium globosum, and find particular application in the degradation of plastic products comprising polyurethane. Esterases or lipases from Arthrobacter sp. or Enterobacter sp. find particular application in the degradation of plastic products comprising polycarbonate.

Suitable cutinases include those from Thielavia terrestris, Thermobifida fusca, Thermobifida cellulosityca, Thermobifida alba, Thermobifida halotolerans, Bacillus subtilis, Humicola insolens and Fusarium solani.

Suitable aryl-acylamidases include that from Nocardia farcinica).

Suitable oligomer hydrolases include the 6-aminohexanoate oligomer hydrolase from Arthrobacter sp.). Suitable amidases include that from Beauveria brongniartii.

### 5.3.2 Ancillary Non-Plasticase Bioremediation Enzymes

It will be appreciated that the PA plastic product may be physically associated with non-plastic contaminants and pollutants, particularly in cases where the PA plastic product forms part of domestic, commercial or industrial mixed waste streams or collected plastic wastes.

It will also be appreciated that contaminants and pollutants may be released during PEase mediated degradation (and/or during treatment with adjunctive (augmentary or complementary) plasticase enzymes). For example, and as explained above, the PA plastic product substrates may further comprise various additives, such as acid scavengers, prodegradants, antioxidants, antistatic agents, flame-retardants, light stabilizers, thermal stabilizers, lubricants, non-slip compounds, plasticizers, pigments, dyes and mineral or organic fillers (including metals, such as cadmium, chromium, lead, mercury, cobalt and zinc), any of which may be released during enzymatic degradation.

Thus, processing of the PA plastic product with one or more ancillary non-plasticase bioremediation enzymes in conjunction with the PAase of the invention (and any adjunctive (augmentary or complementary) plasticase enzymes) may be advantageous, and may reduce or eliminate the production of pollutants during enzymatic degradation using the PAases of the invention.

As used herein, the term ancillary non-plasticase bioremediation enzyme defines an enzyme which enzymatically degrades one or more non-plastic pollutants, and which is not a PAase (as herein defined).

Those skilled in the art will be aware of a large variety of such ancillary non-plasticase bioremediation enzymes, and will be able to select an appropriate bioremediation enzyme (or enzyme cocktail) on the basis of the nature of the PA plastic product (and its physical milieu, for example whether present as part of domestic, commercial or industrial mixed waste stream). The field has been recently reviewed (Karigar and Rao (2011) Enzyme Research Volume 2011, Article ID 805187, 11 pages, doi:10.4061/2011/805187—see in particular Table 1, the contents of which are incorporated herein by reference).

Suitable ancillary non-plasticase bioremediation enzyme therefore include enzymes selected from: (a) oxidoreductases; (b) oxygenases; (c) monooxygenases; (d) dioxygenases; (e) laccases; (f) peroxidases; (g) a hydrolase; (h) a peroxygenase (such as the peroxygenase from Agrocybe aegerita); and (i) combinations of two or more of the foregoing.

The use of oxidoreductases in industrial biotransformation has been recently reviewed by Martinez et al. (2017) 35: 815-831.

Preferred ancillary non-plasticase bioremediation enzymes are hydrolases, for example hydrolases selected from: (a) a lipase; (b) a cellulase; (c) a protease; and (d) combinations of two or more of the foregoing.

### 5.3.3 Ancillary Cellulase, Hemicellulase and Ligninase Enzymes

It will be appreciated that the PA plastic product may be physically associated with lignocellulosic materials, particularly in cases where the PA plastic product forms part of domestic, commercial or industrial mixed waste streams or collected plastic wastes.

Lignocellulose is the most abundant biopolymer on earth. The enzymatic degradation of lignocellulosic biomass (e.g., from virgin biomass, waste biomass or energy crops) to yield fermentable sugars, sugar acids and phenolics is now being developed to provide an alternative to fossil fuels via the industrial production of biofuel (typically bioethanol) and for the production of other valuable bioproducts.

As used herein, the biomass, in this context of the invention, defines any lignocellulosic material. Suitable lignocellulosic materials include grasses (including reed canary grass, common reed, wheat straw, energy grass, elephant grass, switchgrass, cord grass and rye grass), brans and straws (such as rice, rye, wheat, barley, oat, canola and barley brans and straws), oat hulls and spelt, sorghum, rice hulls, sugarcane bagasse, pressed sugarcane stalk, corn fibre, flax, wheat, linseed, fruit pulps, paper pulp, sugar beet pulp, locust bean pulp, cottonseed, groundnut, rapeseed, sunflower, peas, lupins, palm kernel, coconut, konjac, locust bean gum, guar gum, soy beans, distillers grains, spent grain, corncobs, energy cane, cassava peels, cocoa pods, vinasse, wood pulp fibre, sawdust, stover, hard- and softwoods (including for example eucalyptus, birch, willow, aspen, poplar) or any combination thereof.

The term is therefore not to be confused with microbial biomass which is a product of a process of the invention (see Section 7.4, below).

For reviews of this technology, see Sun et al. (2002) Bioresource Technology 83: 1-11; and Pathak and Navneet (2017) Bioresour. Bioprocess. 4:15 DOI 10.1186/s40643-017-0145-9.

The PAase of the invention therefore finds application in the enzymatic transformation of biomass feedstocks which contain PA polymers (for example being present as synthetic PA plastics or as PA plastic products, as described above), when combined with ancillary enzymes which degrade cellulose, hemicellulose and/or lignocellulose.

Such applications are particularly important in cases where the PAase substrate is present as part of a domestic, commercial or industrial mixed waste stream containing lignocellulosic materials (such as garden waste, kitchen waste, agricultural waste, sawdust, wood chips, cardboard, cardboard pulp, paper, paper pulp and non-plastic packaging).

Thus, the processing of biomass containing plastic with the PAase of the invention in combination with ancillary cellulose, hemicellulose and/or ligninase enzymes (and optionally further combined with (a) helper enzymes; and/or (b) adjunctive plasticases (including augmentary and/or complementary plasticases); and/or (c) ancillary non-plasticase bioremediation enzymes, as described in Sections 5.3.1, 5.3.2 (above) and 6.2 (below)) may be advantageous, and may facilitate the production of valuable bioproducts (including biofuels) from mixed waste feedstocks.

Suitable ancillary cellulases, hemicellulases, ligninases include various lignin peroxidases, xylanases, β-xylosidases, arabinofuranosidases, glucanases, glycoside hydrolases and esterases. Exemplary cellulases include endoglucanases of bacterial or fungal origin, including endoglucanases (EC 3.2.1.4). Some xyloglucanases also have endoglucanase activity and so act as cellulases. Other suitable cellulases are disclosed in U.S. Pat. No. 4,435,307 (from Humicola insolens) and EP0495257. Suitable mono-component endoglucanases may be obtained from Exidia glandulosa, Crinipellis scabella, Fomes fomentarius, Spongipellis sp., Rhizophlyctis rosea, Rhizomucor pusillus, Phycomyces nitens, and Chaetostylum fresenii, Diplodia gossypina, Microsphaeropsis sp., Ulospora bilgramii, Aureobasidium sp., Macrophomina phaseolina, Ascobolus stictoides, Saccobolus dilutellus, Peziza, Penicillium verruculosum, Penicillium chrysogenurn, and Thermomyces verrucosus, Trichoderma reesei aka Hypocrea jecorina, Diaporthe syngenesia, Colletotrichum lagenarium, Xylaria hypoxylon, Nigrospora sp., Nodulisporum sp., and Poronia punctata, Cylindrocarpon sp., Nectria pinea, Volutella colletotrichoides, Sordaria fimicola, Sordaria macrospora, Thielavia thermophila, Syspastospora boninensis, Cladorrhinum foecundissimurn, Chaetomium murorum, Chaetomium virescens, Chaetomium brasiliensis, Chaetomium cunicolorum, Myceliophthora thermophila, Gliocladium catenulatum, Scytalidium thermophila, Acremonium sp Fusarium solani, Fusarium anguioides, Fusarium poae, Fusarium oxysporum ssp. lycopersici, Fusarium oxysporum ssp. passiflora, Humicola nigrescens, Humicola grisea, Fusarium oxysporum, Thielavia terrestris and Humicola insolens.

Commercially available xyloglucanases include is Whitezyme® (Novozymes A/S).

Commercially available cellulases include Celluclast® (from Trichoderma reesei), Celluzyme® (from Humicola insolens). Commercially available endoglucanases include Carezyme®, Renozyme®, Endolase® and Celluclean®, KAC-500(B)™, Clazinase™, Puradax™ EG L and Puradax HA.

Suitable glycoside hydrolase family 61 (GH61) enzymes are described in WO2005/074647, WO2008/148131 and WO 2011/035027 (from Thielavia terrestris), WO2005/074656 and WO2010/065830 (from Thermoascus aurantiacus), WO2007/089290 and WO2012/149344 (from Trichoderma reesei), WO2009/085935, WO2009/085859, WO2009/085864 and WO2009/085868 (from Myceliophthora thermophila), WO2010/138754 (from Aspergillus fumigatus), WO2011/005867 (from Penicillium pinophilum), WO2011/039319 (from Thermoascus sp.), WO2011/041397 (from Penicillium sp.), WO2011/041504 (from Thermoascus crustaceus), WO2012/030799 (from Aspergillus aculeatus), WO2012/113340 (from Thermomyces lanuginosus), WO2012/122477 (from Aurantiporus alborubescens, Trichophaea saccata and Penicillium thomii), WO2012/135659 (from Talaromyces stipitatus), WO2012/146171 (from Humicola insolens), WO2012/101206 (from Maibranchea cinnamomea, Talaromyces leycettanus, and Chaetomium thermophilum), WO2013/043910 (from Acrophialophora fusispora and Corynascus sepedonium).

### 5.3.4 Microbial Digestion

As explained above, biotic processing includes contacting the PA plastic product with one or more microorganisms. The microorganism(s) may express and/or secrete one or more of the enzymes (adjunctive (augmentary or complementary) plasticase enzymes and/or ancillary non-plasticase bioremediation enzymes) described above. Alternatively, or in addition, the microorganism(s) may produce one or more mediator compounds or cofactors which facilitate the degradative activity of one or more of said enzymes. In preferred embodiments, a microbial consortium comprising at least 2, 3, 4, 5, 6, 7, 8, 9 10 different microbial organisms are used.

Those skilled in the art will be aware of a large variety of suitable microorganisms, and will be able to select accordingly on the basis of the nature of the PA plastic product (and its physical milieu, for example whether present as part of domestic, commercial or industrial mixed waste stream). The field has been recently reviewed (Pathak and Navneet (2017) Bioresour. Bioprocess. 4:15 DOI 10.1186/s40643-017-0145-9; Varjani and Upasani (2017) International Biodeterioration & Biodegradation 120 71e83; Varjani (2017) Bioresource Technology 223: 277-286 (see in particular Table 1, which is incorporated herein by reference); Ho et al. (2018) Critical Reviews in Biotechnology, 38:2: 308-320 (see in particular Table 4, which is incorporated herein by reference); Jaiswal et al. (2020) Environmental Technology & Innovation 17: 100567 (see in particular Tables 1 and 2, which are incorporated herein by reference).

In embodiments where biotic processing involving microbial digestion is employed, the PA plastic product may be contacted with a culture medium containing the microorganism(s) (inoculum) together with a carbon source (such as glucose) and a nitrogen source (for example, an organic nitrogen source selected from peptone, meat extract, yeast extract and corn steep liquor), or an inorganic nitrogen source (e.g. ammonium sulfate or ammonium chloride). The culture medium may further comprise inorganic salts (e.g. sodium, potassium, calcium, magnesium, sulfate, chlorine, phosphate salts) and may be supplemented with trace components such as vitamins and amino acids.

In preferred embodiments, the microorganism is selected from the following genera: Achromobacter, Acinetobacter, Actinomadura, Alcaligines, Alcanivorax, Amycolatopsis, Aneurinibacillus, Archaeoglobus, Aromatoleum, Arthrobacter, Aspergillus, Azoarcus, Actinomadura, Bionectria, Isaria, Bacillus, Pseudomonas, Fusarium, Beauveria, Brevibacillus, Brevibacterium, Brevundimonas, Candida, Chaetomium, Citrobacter, Cladosporium, Comamonas, Coriolus, Coryneformes, Corynebacterium, Cunninghamella, Curvularia, Cycloclasticus, Delftia, Desulfococcus, Desulfosarcina, Dictyoglomus, Diplococcus, Engyodontium, Enterobacter, Exiguobacterium, Flavobacterium, Gliocladium, Gordonia, Halomonas, Hansenula, Kibdelosporangium, Klebsiella, Kluyveromyces, Leptothrix, Listeria, Marinobacter, Microbacterium, Micrococcus, Moraxella, Mortierella, Mucor, Mycobacterium, Nocardia, Ochrobactrum, Oleispira, Paecylomyces, Paenibacillus, Penicillium, Phanerochaete, Pleurotus, Proteobacterium, Proteus, Pseudomonas, Pseudozyma, Pullularia, Rahnella, Ralstonia, Rhizopus, Rhodococcus, Sphingomonas, Saccharomyces, Serratia, Sphingobacterium, Sphingomonas, Streptomyces, Staphylococcus, Stenotrophomonas, Streptococcus, Talaromyces, Thalassolituus, Thermomonospora, Trametes, Trichoderma, Tritirachium, Vibrio, Xanthomonas and combinations of two or more of the foregoing.

Exemplary species include Aspergillus oryzae, Humicola insolens, Penicillium citrinum, Fusarium solani and Thermobifida cellulolysitica (which synthesize and secrete a cutinase). Yet further examples include Candida antarctica, Thermomyces lanuginosus, Burkholderia sp. and Triticum aestivum (which synthesize a lipase which depolymerizes PET).

Other examples include Amycolatopsis sp. K104-1 and K104-2, Tritirachium album ATCC 22563, Paenibacillus amylolyticus TB-13, Kibdelosporangium aridum JCM 15 7912, Saccharothrix waywayandensis JCM 9114, Amycolatopsis orientalis IFO 12362 and Actinomadura keratinilytica T16-I (which find application in the degradation of plastic products containing PLA in particular). Other examples include Aspergillus fumigatus NKCM1706 and Bionectria ochroleuca BFM-XI (which find particular application in the degradation of plastic products containing PBS). Yet other examples include Thermomonospora fusca K13g and K7a-3, and Isaria fumosorosea NKCM1712 (which find particular application in the degradation of plastic products containing PBAT). Bjerkandera adusta produces a manganese peroxidase and finds particular application in the degradation of plastic products containing polyamide polymers.

### 5.3.5 Insects and Insect Larvae

As explained above, biotic processing includes contacting the PA plastic product with one or more insects or insect larvae. The insects/larvae may harbour (for example in the gut) one or more of the microorganisms described above, which may in turn express and/or secrete one or more of the enzymes (adjunctive (augmentary or complementary) plasticase enzymes and/or ancillary non-plasticase bioremediation enzymes) described above, so enabling the insects/insect larvae to degrade the PA plastic product.

Suitable insects and insect larvae are described in WO2014/067081, and they include members of the Coleoptera and Lepidoptera orders, and so include, without limitation, the suborders Adephaga, Archostemata, Myxophaga and Polyphaga. Example of the Polyphaga suborder include Bostrichiformia, Cucujiformia, Elateriformia, Scarabeiformia, and Staphyliniformia. Examples of the Cucujiformia infraorder include the Chrysomeloidea, Cleroidea, Cucujoidea, Curculionoidea, Lymexyloidea and Tenebrionoidea. Examples of the Tenebrionoidea superfamily include Aderidae, Anthicidae, Archeocrypticidae, Boridae, Chalcodryidea, Ciidae, Melandryidea, Meloidae, Mordellidae, Mycetophagidae, Mycteridae, Oedemeridae, Perimylopidae, Prostomidae, Pterogeniidae, Pyrochroidae, Pythidae, Rhipiphoridae, Salpingidae, Scraptiidea, Stenotrachelidae, Synchroidae, Tenebrionidae, Tetratomidae, Trachelostenidae, Trictenotomidae, Ulodidae and Zopheridae. Examples of the Tenebrionidae family include subfamily members Alleculinae, Coelometopinae, Diaperinae, Lagriinae, Palorinae, Phrenapatinae, Pimeliinae, Tenebrioninae and Zolodininae. Examples of the Tenebrionidae family also include genus members Adelina, Alphitobius, Amarygmus, Ammophorus, Archeoglenes, Blapstinus, Diaperis, Epantius, Eutochia, Gnathocerus, Gonocephalum, Hypophloeus, Latheticus, Lobometopon, Lyphia, Mesomorphus, Myrmechixenus, Palembus, Palorus, Phaleria, Platydema, Sciophagus, Tagalus, Tenebrio, Tribolium and Uloma. Preferred insects are Tenebrio molitor, Zophobas morio, Galleria mellonella and Podia interpunctella.

### 6. Compositions of the Invention

### 6.1 General Considerations

The amount of PAase present in the compositions of the invention can be readily determined by the skilled person by reference inter alia to the nature of the nature and (relative amounts) of PA polymer substrate9s) and/or the number of any additional enzymes present in the composition. For example, in preferred embodiments, the PAase of the invention is used in an amount up to 5% by weight of the PA polymer substrate, more preferably up to 1%, even more preferably up to 0.1%, and yet more preferably up to 0.05% by weight of the PA polymer substrate. For example, the amount of PAase of the invention may be in a range of 0.001% to 5% by weight of the PA polymer substrate, preferably in the range of 0.001% to 1%, more preferably in the range of 0.001% to 0.1%, and even more preferably in the range of 0.001% to 0.05% by weight of the PA polymer substrate.

The amount of PAase present in the compositions of the invention are therefore selected to conveniently facilitate the above dosing ratios, and so in preferred embodiments, the compositions preferably comprise at least 0.01%, 0.1%, 1%, 5%, 10%, 15%, 20%, 25% or 30% by weight PAase, based on the total weight of the composition. In some embodiments, the compositions preferably comprise up to 0.01%, 0.1%, 1%, 5%, 10%, 15%, 20%, 25% or 30% by weight PAase, based on the total weight of the composition. In some preferred embodiments, the compositions comprise between 0.1% and 50%, or between 0.1% and 40%, or between 0.1% and 30%, or between 0.1% and 20%, or between 0.1% and 10%, PAase (based on the total weight of the composition).

The PAase is preferably present in the compositions in isolated or purified form. For instance, the PAase may be expressed, derived, secreted, isolated, or purified from a microorganism (for example from Ralstonia pickettii). The PAase may be purified by biochemical techniques known in the art.

The compositions comprising the PAase are preferably substantially anhydrous, for example in the form of granules or a powder. Anhydrous compositions may be prepared by any suitable technique known to those skilled in the art, including lyophilisation, freeze-drying, spray-drying, supercritical drying, down-draught evaporation, thin-layer evaporation, centrifugal evaporation, conveyer drying, fluidized bed drying, drum drying and combinations of the foregoing techniques. Preferably, The PAase is freeze-dried or spray-dried.

In the case of substantially anhydrous compositions, the PAase preferably further comprises an inert bulking agent selected from starch, dextrin (e.g. cyclodextrin and/or maltodextrin), sugars (e.g. sorbitol, trehalose and/or lactose), carboxymethylcellulose, poly-electrolytes, trehalose, and compatible solutes selected from proline, betaine, glutamate and glycerol.

In preferred embodiments, the PAase is in a stabilized form. For example, it may be in the form of a cross-linked enzyme (CLE), crystallized cross-linked enzyme (CLEC) and/or an aggregated cross-linked enzyme (CLEA) (see e.g. Velasco-Lozano et al. (2015) Biocatalysis 1: 166-177 for a review of suitable technologies).

The PAase may be stabilized by coating. In such cases, the compositions comprising the PAase of the invention may take the form of coated enzyme granules (for example as described in WO00/01793, WO2004/003188, WO2004/067739, WO99/32595, WO2006/034710 and WO2007/044968.

In other embodiments, the PAase is in the form of a solution, suspension, emulsion or dispersion, optionally in the form of an aqueous solution, suspension, emulsion or dispersion. In certain embodiments, the PAase may be present in the composition in an immobilized from, for example being bound to cell membranes, within lipid vesicles, or attached to solid supports, matrices or particles (for example selected from glass beads and mineral, polymer or metallic particles or matrices).

The PAase may be present in the compositions together with one or more excipients. Such excipients include buffers, preservatives (for example selected from sodium benzoate, sodium sorbate and sodium ascorbate), bulking, protective and/or stabilizing agents (for example selected from carboxymethylcellulose, starch, dextrin, arabic gum, salts, sugars (e.g. sorbitol, trehalose and/or lactose), glycerol, polyethyleneglycol, polypropylene glycol, propylene glycol, sequestering agent (e.g. EDTA), reducing agents, amino acids, carriers (such as an aqueous or organic solvent or suspension medium), poly-electrolytes, trehalose, and compatible solutes such as proline, betaine, glutamate and/or glycerol.

The PAase may also be present in the compositions together with various reactants and/or cofactors. Such reactants and cofactors may be used by the PAase of the invention, or by any adjunctive (augmentary or complementary) or ancillary enzyme that may also be present in the composition.

The PAase can be conveniently produced by recombinant techniques using a cellular expression system (see e.g. Johnsson et al. (1997) J Biol Chem 272: 2834-2840; Zámocký et al. (2012) Biochimie 94: 673-683; Doyle and Smith (1996) Biochem J 315 (Pt 1): 15-19).

Any suitable expression system may be used, including those involving prokaryotic cells and eukaryotic cells. Suitable methodologies have been recently reviewed by Gomes et al. (2016) Advances in Animal and Veterinary Sciences 4(4): 346-356.

Suitable prokaryotic cells may be selected from: (a) Escherichia coli; (b) Bacillus subtilis; (c) Bacillus megaterium; (d) Bacillus licheniformis; (e) Lactococcus lactis; (f) Ralstonia eutropha; (g) Pseudomonas spp.; (h) Ralstonia pickettii; and (i) Corynebacterium spp.

Particularly preferred as a heterologous host are E. coli strains engineered to export the PAase to the cell surface (or extracellularly) using the AIDA-I autotransporter (by control of the the status of the protease ompT (+/−)), as described in Fleetwood et al. (2014) Microb Cell Fact 13:179; Jose and Meyer (2007) Microbiol Mol Biol Rev 71: 600-619.

Suitable eukaryotic cells may be selected from: (a) yeasts; (b) fungi; (c) insect cells; (d) mammalian cells; and (e) plant cells. Suitable yeast cells may be selected from: (a) Saccharomyces cerevisiae; (b) Hansenula polymorpha; (c) Komagatella phaffii; (d) Candida biodini; and (e) Schizosaccharomyces pombe. Suitable fungal cells may be selected from: (a) a filamentous fungus, for example Aspergillus spp. and Trichoderma spp., and (b) Myceliophthora thermophila (e.g. the C1 expression system).

It will be noted that the above considerations are generally applicable, and so apply mutatis mutandis to any other enzymes that may be present in the compositions of the invention. The above considerations therefore apply to each of the other enzymes described in Section 5.3.1, 5.3.2, 5.3.3 (above) and 6.2 (below), and so extend to: (a) helper enzymes; and/or (b) adjunctive plasticases (including augmentary and/or complementary plasticases); and/or (c) ancillary non-plasticase bioremediation enzymes; and/or (d) ancillary cellulases, hemicellulases and/or ligninases, if present in the compositions of the invention.

The invention contemplates various compositions comprising the PAase of the invention, including the following:
Enzyme system compositions comprising the PAase of the invention and one or more helper enzymes for generating hydrogen peroxide.
Enzyme compositions comprising a PAase of the invention and a plastic binding protein.
Plasticase cocktail compositions comprising the PAase of the invention and one or more adjunctive plasticase enzymes.
Waste remediation enzyme cocktail compositions comprising the PAase of the invention and one or more ancillary non-plasticase bioremediation enzymes.
Lignocellulose biomass enzyme cocktail compositions comprising the PAase of the invention and one or more ancillary cellulose, hemicellulose and/or ligninase enzymes.
Recombinant cellular hosts expressing (and optionally secreting) the foregoing enzyme compositions.
Reaction mix compositions comprising the PAase of the invention in combination with a PA polymer substrate.
KatG prodegradant compositions.
Composite KatG plastics comprising a PA polymer and a KatG prodegradant.
Autodegradative PA polymers comprising the KatG prodegradant.

Considering each in more detail in turn:

### 6.2 PAase Enzyme System Compositions

The invention provides enzyme system compositions comprising the PAase of the invention and one or more helper enzymes for generating hydrogen peroxide, as described below.

The PAase of the invention catalyses the following reaction:

PA donor+H₂O₂→oxidized PA donor+2 H₂O

Thus, the reaction in the case of a (simplified) PE plastic (where the plastic polymer is represented as CH₃CH₃ but is part of a longer chain of a —CH₂CH₂— backbone) may be represented as:

H₂O₂+CH₃CH₃→HOCH₂CH₂OH+2H

In addition, any further oxidative reactions that may use such hydrogen peroxide may also lead to further oxidised products of the form H—O—O—X (peroxides), HC(═O)—X (aldehydes), HO(O═)C—X (carboxylic acids), and the corresponding homo-di-substituted variants (di-peroxides, dialdehydes, and in particular di-carboxylic acids), and mixed hetero derivatives (mixed acids, aldehydes, alcohols and peroxides, mutatis mutandis) in which the degree of oxidation of the oxidised polyalkenes varies.

In such reactions, the peroxide may be conveniently provided by one or more enzymes which catalyse the formation of a peroxide (preferably hydrogen peroxide) which, when coupled with the PAase and PA plastic substrate, supply the hydrogen peroxide required by the PAase for the oxidation of the PA plastic substrate.

Such enzymes can provide a gradual supply of peroxide, maintaining its concentration at (near) stoichiometric levels (so reducing or preventing irreversible peroxide-mediated inactivation of enzymes present in the reaction mix). They are herein defined as helper enzymes, since they function to promote the peroxidase activity of the PAase (without being themselves directly involved in plasticase activity).

Preferred helper enzymes generate hydrogen peroxide via the reduction of molecular oxygen (O₂). Such enzymes, typically oxidases, may act as cascades, for example using formate, methanol or ethanol as the source of reducing equivalents for the reduction of O₂ to H₂O₂.

Particularly preferred helper enzymes include formate oxidase (FOx) and formate dehydrogenase as a multienzymatic in situ hydrogen peroxide generation cascade (as described in Pesic et al. (2018) Z. Naturforsch. 2019; 74(3-4)c:101-104), which may be represented thus:

| | |
|---|---|
| HCOOH + O₂ → H₂O₂ + CO₂ | formate oxidase |
| H₂O₂ + CH₃CH₃ → HOCH₂CH₂OH + 2H | PAase |
| 2H + CO₂ →HCOOH | formate dehydrogenase |

CH₃CH₃ + O₂ → HOCH₂CH₂OH

Another suitable helper system is described by de Santos et al. (2018) ACS Catal 8: 4789-4799, and may be represented as follows:

| | |
|---|---|
| 2CH₃OH + 2O2 → 2HCHO + 2H₂O₂ | methanol oxidase |
| 2 HCHO + H₂O → CH₃OH + HCOOH | formaldehyde dismutase |

CH₃OH + 2O₂ + H₂O → 2H₂O₂ + HCOOH

In a particularly preferred embodiment, the source of reducing equivalent for producing the H₂O₂ is an alcohol, for example methanol, as follows:

| | |
|---|---|
| 2CH₃OH + 2O₂ → 2HCHO + 2H₂O₂ | methanol oxidase |
| 2 HCHO + H₂O → CH₃OH + HCOOH | formaldehyde dismutase |
| HCOOH + O₂ → CO₂ + H₂O₂ | formate oxidase |

CH₃OH + H₂O + 3 O₂ → CO₂ + 3 H₂O₂

The above (almost circular) helper systems find particular application in airlift reactors where the oxygen reactant can be supplied with the pressurized air feed.

Suitable formate oxidase enzymes are described in Doubayashi et al. (2019) J Biochem 166:67-75; Doubayashi et al. (2011) Biosci Biotechnol Biochem 75:1662-1667; Maeda et al. (2009) Biosci Biotechnol Biochem 73:2645-2649; Robbins et al. (2018) Arch Biochem Biophys 643:24-31; Robbins et al. (2018b) Biochemistry 57:5818-5826; Robbins et al. (2017) Biochemistry 56: 3800-3807; Tieves et al. (2019) Angew Chem Int Ed Engl 58: 7873-7877 and Uchida et al. (2007) Appl Microbiol Biotechnol 74: 805-812.

Suitable methanol oxidase enzymes are described in Vonck et al. (2016) PLoS One 11: e0159476 and de Oliveira et al. (2012) Fungal Genet Biol 49: 922-932.

Suitable formaldehyde dismutase enzymes are described in Blaschke et al. (2017) J Biotechnol 241: 69-75; Mason and Sanders (1989) Biochemistry 28: 2160-2168;

Ni et al. (2016) Angewandte Chemie-International Edition 55: 798-801; van der Waals et al. (2016) Chemistry 22: 11568-11573; Yanase et al. (2002) Biosci Biotechnol Biochem 66: 85-91; Yonemitsu and Kikuchi (2018) Biosci Biotechnol Biochem 82: 49-56; Yonemitsu et al. (2016) Biosci Biotechnol Biochem 80: 2264-2270; Kato et al. (1983) Agric Biol Chem 47: 39-46; Hasegawa. et al. (2002) Acta Cryst A8: C102 and Yonemitsu et al. (2016) Biosci Biotechnol Biochem 80: 2264-2270.

The enzyme system compositions of the invention preferably further comprise one or more substrates for the helper enzymes for generating hydrogen peroxide. In preferred embodiments, the one or more substrates comprise methanol and/or ethanol and/or formate.

In a further embodiment, the oxidative power is provided by molecular dioxygen (O₂), including from air, rather than or in addition to hydrogen peroxide, providing products in the same families as described above.

### 6.3 PAase-Plastic Binding Protein Compositions

As used herein, the term plastic binding protein defines a non-enzyme protein that promotes or mediates the adsorption of the PAase of the invention onto a synthetic PA plastic or PA plastic product.

Plastic binding proteins include biosurfactants that adsorb to hydrophobic substances and create an interface between the hydrophobic surface of the plastic and a surrounding hydrophilic aqueous phases. Exemplary biosurfactants include hydrophobins, which are small secreted fungal proteins containing 8 positionally conserved cysteine residues, and a distinct hydrophobic patch. Hydrophobins adsorb to hydrophobic substances and to interfaces between hydrophobic (plastic) and hydrophilic (aqueous medium) phases, and assemble into amphiphilic structures and reduce the interface energy between the plastic and the PAase (Takahashi et al. (2005) Mol. Microbiol. 57, 1780-1796; Espino-Rammer et al. (2013) AEM 79: 4230-4238). Preferably, the hydrophobins are rigid enough to keep the hydrophobic patch exposed when fused to a protein (Paananen et al. (2013) Soft Matter 9: 1612-1619).

Other plastic binding proteins include “disrupting” or “amorphogenesis inducing” proteins (reviewed in Arantes and Saddler (2010) Biotechnol. Biofuels 3: 4). They include disrupting proteins (for example expansins and swollenins) that act by weakening the hydrogen bonds between adjacent polymeric chains of the PA plastic. Examples include the Expansins (Cosgrove (2000) Nature 407 (6802): 321-326), bacterial Expansin-like proteins (Lee et al. (2010) Mol. Cells 29(4): 379-385), fibril forming protein (Banka et al. (1998) World J. Microbiol. Biotechnol. 14(4): 551-560, certain carbohydrate binding modules (Din et al., (1991) Nat. Biotechnol. 9: 1096-1099; Din et al. (1994) Proc. Natl. Acad. Sci. U.S.A. 91 (24): 11383-11387; Gao et al. (2001) Acta Biochimica et Biophysica Sinica 33(1): 13-18), fungal Expansin-like proteins, Loosenin (Quiroz-Castañeda et al. (2011) Microb. Cell Fact. 10: 8) and Swollenins (Gourlay et al. (2012) Biotechnol. Biofuels 5(1): 51; Jäger et al. (2011) Biotechnol. Biofuels 4 (33); Saloheimo et al. (2002) Eur. J. Biochem. 269 (17): 4202-4211 and Verma et al. (2013) PLoS One, 8 (2) (2013), p. e57187).

The invention provides PAase compositions comprising the PAase of the invention and one or more plastic binding proteins (as herein defined).

The plastic binding protein may be provided in admixture with the PAase, but may also be fused to the PAase. The invention therefore contemplates compositions comprising PAase-plastic binding protein fusion proteins (as described in Section 3.2.7, above).

### 6.4 Plasticase Cocktail Compositions

In certain embodiments the PAase of the invention may be used in conjunction with various other enzymes which exhibit degradative activity against one or more synthetic plastic polymers. The invention therefore provides a plasticase cocktail composition comprising a PAase of the invention and one or more adjunctive plasticases.

Preferred adjunctive plasticase enzymes include augmentary plasticases and complementary plasticases.

Such adjunctive enzymes are therefore not essential for the enzymatic degradation of PE polymers by the PEases of the invention, but may find application in cases where the PE polymer is present in admixture with other plastics (for example where the PE polymer is in the form of a heterogeneous PE polymer composition, for example being derived from an unsorted plastic waste stream) or where the nature of the PE polymers and/or the processing conditions may be usefully complemented or augmented by other PEases.

Examples of useful adjunctive enzymes/enzyme classes (including exemplary augmentary plasticases and complementary plasticases) are described in detail in Section 5.3.1 (above).

Preferred complementary plasticase enzymes include those which degrade one or more of the additional plastic substrates listed in Section 4.4 (above).

### 6.5 Ancillary Bioremediation Enzyme Cocktail Compositions

The invention contemplates compositions comprising the PAase of the invention and an ancillary non-plasticase bioremediation enzyme. Such compositions preferably further comprise: (a) an enzyme system composition as described in Section 6.2 (above); and/or (b) one or more adjunctive (e.g. augmentary and/or complementary) plasticase enzymes (as described in Section 6.4, above).

As used herein, the term ancillary non-plasticase bioremediation enzyme defines an enzyme which enzymatically degrades one or more non-plastic pollutants, and which is not a PAase (as herein defined).

Such ancillary enzymes are therefore not essential for the enzymatic degradation of PE polymers by the PEases of the invention, but may find application in cases where the PE polymer is present in admixture with other contaminants (for example where the PE polymer is present along with other environmental pollutants, for example being derived from a contaminated landfill sample).

The ancillary non-plasticase bioremediation enzyme compositions of the invention find application in the reduction or elimination of pollutants present during (and/or produced by) enzymatic degradation using the PAases of the invention, as explained in Section 5.3.2 (above).

Those skilled in the art will be aware of a large variety of such ancillary non-plasticase bioremediation enzymes, and will be able to select an appropriate bioremediation enzyme (or enzyme cocktail) on the basis of the nature of the PA plastic product (and its physical milieu, for example whether present as part of domestic, commercial or industrial mixed waste stream). Suitable ancillary non-plasticase bioremediation enzyme therefore include enzymes selected from: (a) oxidoreductases; (b) oxygenases;

(c) monooxygenases; (d) dioxygenases; (e) laccases; (f) peroxidases; (g) a hydrolase; (h) a peroxygenase (such as the peroxygenase from Agrocybe aegerita); and (i) combinations of two or more of the foregoing. Preferred ancillary non-plasticase bioremediation enzymes are hydrolases, for example hydrolases selected from: (a) a lipase; (b) a cellulose; (c) a protease; and (d) combinations of two or more of the foregoing.

Suitable ancillary bioremediation enzymes are described in detail in Section 5.3.2 (above).

Preferred ancillary bioremediation enzymes include those which degrade one or more plastic additives, such as acid scavengers, prodegradants, antioxidants, antistatic agents, flame-retardants, light stabilizers, thermal stabilizers, lubricants, non-slip compounds, plasticizers, pigments, dyes and mineral or organic fillers (including metals, such as cadmium, chromium, lead, mercury, cobalt and zinc). These are described in more detail in Section 4.4 (above).

### 6.6 Ancillary Cellulases, Hemicellulases and Lignocellulases

The invention contemplates compositions comprising the PAase of the invention and an ancillary cellulase, hemicellulase and/or lignocellulase.

Such compositions preferably further comprise: (a) an enzyme system composition as described in Section 6.2 (above); and/or (b) one or more adjunctive (e.g. augmentary and/or complementary) plasticase enzymes (as described in Section 6.3, above) and/or (c) one or more ancillary non-plasticase bioremediation enzymes (as described in Section 6.4, above).

Suitable cellulase, hemicellulase and lignocellulase enzymes are described in Section 5.3.3 (above).

### 6.7 Cellular Expression System Compositions

The invention provides a recombinant cellular host expressing the PAase of the invention, preferably together with one or more of the following enzymes/proteins:
(a) one or more of the helper enzymes described in Section 6.2 (above); and/or
(b) one or more of the plastic binding proteins described in Section 6.3 (above); and/or
(c) one or more of the adjunctive (i.e. augmentary and/or complementary) plasticase enzymes referenced in Section 6.4 (above); and/or
(d) one or more of the ancillary non-plasticase bioremediation enzymes referenced in Section 6.5 (above); and/or
(e) one or more of the ancillary cellulases, hemicellulases and lignocellulases enzymes referenced in Section 6.6 (above).

Particularly preferred are recombinant cellular hosts expressing the PAase of the invention together with one or more helper enzymes. More particularly preferred are recombinant cellular hosts expressing the PAase of the invention together with: (a) one or more helper enzymes; and (b) one or more adjunctive plasticase enzymes.

The PAase and one or more other enzymes/proteins are preferably heterologous to the cellular host. Thus, in preferred embodiments the invention contemplates a recombinant cellular host expressing a PAase of the invention together with one or more helper enzymes, wherein both the PAase and the helper enzyme(s) are heterologous to said host.

In more preferred embodiments the invention contemplates a recombinant cellular host expressing a PAase of the invention together with: (a) one or more helper enzymes; and (b) one or more adjunctive plasticase enzymes, wherein the PAase, helper enzyme(s) and adjunctive plasticase enzyme(s) are all heterologous to said host. Suitable cellular hosts are described in Section 2.2 (above).

The invention also contemplates nucleic acid encoding the PAase of the invention together with one or more of the enzymes/proteins (a)-(e) (above). The nucleic acid may be DNA or RNA. Preferably, the nucleic acid is polycistronic DNA.

The invention also contemplates vectors (e.g. an expression vector) comprising the nucleic acid of the invention. The nature of the vector is not critical to the invention. Any suitable vector may be used, including plasmid, virus, bacteriophage, transposon, minichromosome, liposome or mechanical carrier.

Preferred expression vectors of the invention are DNA constructs suitable for expressing DNA which encodes the PAase of the invention and other enzyme(s)/protein(s) in one of the cellular hosts described in Section 2.2 (above). Preferred expression vectors include: (a) a regulatory element (e.g. a promoter, operator, activator, repressor and/or enhancer), (b) a structural or coding sequence which is transcribed into mRNA and (c) appropriate transcription, translation, initiation and termination sequences. They may also contain sequence encoding any of various tags (e.g. to facilitate subsequent purification of the expressed protein, such as affinity (e.g. His) tags) and/or signal sequence(s) (e.g. to direct the secretion of the encoded products).

Particularly preferred are vectors which comprise an expression element or elements operably linked to the DNA of the invention to provide for expression thereof at suitable levels. Any of a wide variety of expression elements may be used, and the expression element or elements may for example be selected from promoters, enhancers, ribosome binding sites, operators and activating sequences. Such expression elements may comprise an enhancer, and for example may be regulatable, for example being inducible (via the addition of an inducer).

The term operably linked refers to a condition in which portions of a linear DNA sequence are capable of influencing the activity of other portions of the same linear DNA sequence. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; a ribosome binding site is operably linked to a coding sequence if it is positioned so as to permit translation.

The vector may further comprise a positive selectable marker and/or a negative selectable marker. The use of a positive selectable marker facilitates the selection and/or identification of cells containing the vector.

### 6.8 Reaction Mix Compositions

The invention provides a reaction mix composition for use in the reactors of the invention, wherein the mix comprises a PAase of the invention in combination with a PA polymer.

The PA polymer in the reaction mix is preferably a PA polymer substrate as described in Section 4.1 (above). More preferred are the PE, PP, PVC, PS and CEP polymer substrates described in Section 4.2 (above). Yet more preferred are reaction mixes comprising PA polymers selected from the synthetic PA plastic substrates described in Section 4.3 (above).

Also preferred are reaction mixes comprising PA polymers in the form of the PA plastic products described in Section 4.4 (above). In such embodiments, the PA plastic product is preferably processed before incorporation into the reaction mix.

In preferred embodiments, the reaction mix comprises PA polymers in the form of PA plastic products processed as described in Section 5 (above). The reaction mix may further comprise an aqueous solvent, and may be is in the form of an aqueous solution, suspension or dispersion. The reaction mix may further comprise one or more buffers.

The reaction mix may further comprise one or more of the other enzyme compositions described above), and so include: (a) helper enzymes; and/or (b) adjunctive plasticases (including augmentary and/or complementary plasticases); and/or (c) ancillary non-plasticase bioremediation enzymes; and/or (d) ancillary cellulases, hemicellulases and/or ligninases.

The reaction mix may further comprise a plastic binding protein as herein defined (and described in Section 6.3, above). The reaction mix preferably further comprises oxygen and/or methanol and/or ethanol and/or formate.

The reaction mix also preferably comprises a surfactant. Surfactants may promote access of the PAase to the PA plastic product, for example by facilitating contact of the PAase with hydrophobic surfaces of the plastic product. Preferred surfactants include non-ionic surfactants. Other preferred surfactants include Tween 80, Tween 20 and CHAPSO (which have been shown to promote the degradation of PE by a partially purified MnP (as described by liyoshi et al. (1998) op. cit. and Ehara et al. (2000) op. cit.).

The reaction mix also preferably comprises one or more cofactors. In preferred embodiments, the one or more cofactors comprise haem and/or iron and/or flavin mononucleotide (FMN) and/or FAD(H₂) and/or NAD(H).

### 6.9 Composite Plastics with KatG Prodegradant Additive

### 6.9.1 Degradable Plastics

Various different physical, chemical and/or biochemical approaches have been developed to increase the rate at which synthetic plastic products are degraded after disposal, either in the environment and/or as feedstock in a waste processing system. For example, plastic articles can be made from a mix of polyalkenes and biodegradable polymers, which may be natural (e.g., starch or cellulose) or synthetic.

However, these so-called “biodegradable” plastics are expensive, not easily processed, and typically have relatively poor mechanical properties. Moreover, only the biodegradable component of the polymer mixture is degraded, the polyalkene polymers remaining essentially intact.

Oxo-degradable plastics (mainly based on polyethylene) contain abiotic prodegradant additives which catalyse oxidation reactions to weaken and fragment the plastic. They are sometimes referred to as “thermofragmentable” or “photofragmentable” plastics. The most common abiotic prodegradants are the transition metals Fe, Co or Mn, introduced in trace quantities into the polymer product as salts, fatty acid esters, amides, dithiocarbamates, ferrocene and metal oxides. Other approaches include the introduction of organic groups that reduce stability in light, heat or moisture (such as carbonyl groups, oxo-hydroxy groups, unsaturated alcohols and esters, benzophenones, γ-pyrones, β-diketones, polyisobutylene, amines and peroxides).

However, there is little evidence that oxo-degradable polymers are completely degraded in the environment: rather, it appears that oxo-degradable polymers become merely fragmented, in which state they can persist in the environment in an irrecoverable form as microplastics. Microplastic pollution is now recognized as a growing ecological threat, especially in marine ecosystems.

### 6.9.2 KatG Enzymes as a Novel Class of Biotic Prodegradants

The KatG/EC 1.11.1.21 polyalkenase enzymes of the invention (and/or microorganisms expressing and/or secreting them) find application as novel biotic prodegradants in the production of a new class of autodegradable plastics in which a KatG prodegradant is incorporated into the plastic (for example as inclusions or distributed throughout the bulk plastic matrix as a (mono- or poly-) dispersed phase), or disposed thereon as a surface coating.

Surface coating may be particularly preferred in embodiments where the autodegradable plastic is in the form of a plastic container (for example, a bottle, bag, box, blister pack or sachet), when the KatG prodegradant may be advantageously present as an outer coating.

In this context, and as used herein, the term KatG prodegradant is used to define a composition comprising a KatG/EC 1.11.1.21 polyalkenase enzyme of the invention (and/or a microorganism expressing and/or secreting a KatG/EC 1.11.1.21 polyalkenase enzyme), for use as a biotic prodegradant plastic additive. Plastic polymers combined with (e.g. containing or coated with) the KatG prodegradant (as defined above) are herein referred to as composite KatG plastics, while PA polymers combined with (e.g. containing or coated with) a KatG prodegradant (as defined above) are herein referred to as autodegradative PA polymers.

The KatG prodegradant PAase enzyme may be present in the form of an inactive proenzyme which contains one or more cleavage sites for generating an active PAase. Such cleavage sites may be targeted by enzymes (e.g. hydrolase) or chemicals (e.g. acids), which may then serve as activating agents (see Section 6.9.7, below).

The KatG prodegradant may further comprise one or more helper enzymes (as herein defined). In such embodiments, the helper enzymes preferably form part of an enzyme system for generating hydrogen peroxide, and so may further comprise one or more substrates for the helper enzymes for generating hydrogen peroxide.

In preferred embodiments, the one or more helper enzymes comprise one or more helper enzymes selected from: (a) formate oxidase; (b) formate dehydrogenase; (c) formaldehyde dismutase; and (d) methanol oxidase. In preferred embodiments, the helper enzymes comprise formate oxidase and formate dehydrogenase. In another preferred embodiment, the helper enzymes comprise methanol oxidase, formaldehyde dismutase and formate oxidase. In such embodiments, suitable substrates include methanol and/or ethanol and/or formate.

The KatG prodegradant may further comprise one or more adjunctive plasticase enzymes (as herein defined). Alternatively, or in addition, the KatG prodegradant may further comprise one or more ancillary non-plasticase bioremediation enzymes (as herein defined).

### 6.9.3 KatG Prodegradant Compositions

The KatG prodegradants described above may take any physical form.

In preferred embodiments, the KatG prodegradant composition is in the form of a solid. Preferred solid forms are granules and powders. In such cases, one or more of the enzymes may be in the form of a cross-linked enzyme (CLE), crystallized cross-linked enzyme (CLEC) and/or an aggregated cross-linked enzyme (CLEA).

KatG prodegradant compositions may be freeze-dried or spray-dried. In such embodiments, they may further comprise an inert bulking agent selected from starch, lactose, carboxymethylcellulose, poly-electrolytes, trehalose, and compatible solutes such as proline, betaine, glutamate and/or glycerol.

KatG prodegradant compositions may also be in the form of a liquid. For example, the KatG prodegradant compositions may take the form of a solution, suspension, emulsion or dispersion. In such cases, the solution, suspension, emulsion or dispersion is preferably aqueous, and may comprise one or more carriers. Such carriers may be selected from a natural gums, including arabic gum, guar gum, tragacanth gum and karaya gum. The solution, suspension, emulsion or dispersion may also be an organic solvent, and may comprise one or more carriers. Such organic solvent-based liquids KatG prodegradant compositions may find particular application in the production of composite KatG plastics which involve mixing with molten thermoplastics (see below).

Polymers having KatG prodegradant inclusions in liquid form may, counter-intuitively, exhibit improved physical properties (for example, improved stiffness)—see Style et al. (2015) Nature Physics 11: 82-87. Suitable formulations and methods which may be adapted for the use of KatG prodegradant polymer inclusions in liquid form are described in WO2019043145.

### 6.9.4 Composite KatG Plastics

The invention therefore provides a plastic polymer composite comprising a KatG prodegradant (as defined above). In preferred embodiments, the composite KatG plastics are composite KatG thermoplastics.

In preferred embodiments, the plastic polymer composite comprises a PA polymer in combination with the KatG prodegradant. In such embodiments, the PA polymer composite is an autodegradative PA polymer, since the KatG prodegradant can be activated after disposal, either in the environment and/or when used as feedstock in a waste processing system, to degrade the PA polymer.

Suitable PA polymers include the PE polymers described above, as well as the PA polymers (which may be considered as substituted PE polymers) PP, PS and PVC (also described above).

In other preferred embodiments, the plastic polymer composite does not comprise a PA polymer, but rather comprises a plastic polymer of a different class (for example, a polymer with a heteroatomic backbone). In such embodiments, the plastic polymer composite may not itself be autodegrading, but rather serves as a source of KatG/EC 1.11.1.21 polyalkenase enzyme for the degradation of co-localized PA polymers present in the environment or in the mixed-plastics feedstock of a waste processing system.

The plastic polymer of a different class may be a non-PA polymer, for example being a polymer having a heteroatomic backbone, such as PET or PUR. The plastic polymer of a different class is preferably a thermoplastic. Examples of non-PA polymers which may be used according to this aspect of the invention include those listed in US2016333147, WO2016198652, WO2016198650, WO2016097325 and WO2019043145 (the content of which is hereby incorporated by reference).

In embodiments where the plastic polymer composite comprises a PA polymer in combination with a KatG prodegradant which further comprise one or more adjunctive plasticase enzymes, the PA polymer composite is not only autodegradative but also serves as a source of a plasticase enzyme cocktail for the degradation of other co-localized plastic polymers, including non-PA polymers, present in the environment or in the mixed-plastics feedstock of a waste processing system.

In embodiments where the plastic polymer composite comprises a PA polymer in combination with a KatG prodegradant which further comprise one or more ancillary non-plasticase bioremediation enzymes, the PA polymer composite is autodegradative and also serves as a source of a bioremediating enzyme cocktail for the degradation of other co-localized pollutants present in the environment or in the feedstock of a waste processing system.

### 6.9.5 Production of Composite KatG Plastics

Incorporation of KatG prodegradant into a polymer can be achieved by a wide variety of suitable techniques known to those skilled in the art. For example, those skilled in the art will be able to select and adapt from the methods described in US2016333147, WO2016198652, WO2016198650, WO2016097325 and WO2019043145 (the content of which is hereby incorporated by reference).

In preferred embodiments, the KatG prodegradant is incorporated by dispersion into the liquid phase of the polymer such that the dispersed material is retained within the polymer matrix after solidification. Preferably, the dispersed material is monodisperse, but in some embodiments the dispersed material may be polydisperse.

Thus, in the preferred case where the polymer plastic is a thermoplastic, the composite KatG plastics of the invention may be produced by a process comprising the step of co-extruding the polymer with the KatG prodegradant at the glass transition temperature (see e.g. Karakolis et al. (2019) Environ Sci Tech Let 6: 334-340) or at a temperature at which the polymer is in a partially or completely molten state. In such cases, the KatG prodegradant and polymer are mixed at a temperature between the glass transition temperature and the melting point of the polymer, or alternatively at its melting point. In particular embodiments, they are mixed at a temperature between 80° C. and 250° C., preferably between 120° C. and 210° C.

Mixing may be achieved by any means, but is conveniently performed using extrusion, twin screw extrusion, single screw extrusion, injection-moulding, casting, thermoforming, rotary moulding, compression, calendering, ironing, coating, stratification, expansion, pultrusion, extrusion blow-moulding, extrusion-swelling, compression-granulation or water-in-oil-in-water double emulsion evaporation. Suitable extrusion means are described in WO2016198796 (the content of which is hereby incorporated by reference).

### 6.9.6 Composite KatG Plastic Article

The composite KatG plastics of the invention may be used to produce any of a wide range of plastic articles. Preferred are moulded plastic articles. The plastic articles may be characterized by physical conformations selected from sheets, films, tubes, pipes, straws, rods, ropes, strings, lines, nets, reticulated sheets, three dimensional regular shapes, blocks, sheaths, fibres, membranes, woven and non-woven textiles, bags, containers, bottles, capsules, packaging, discs, microspheres, (electro)mechanical components, clothing, single-use coffee cups, single-use coffee capsules, building components, Lego® bricks, coatings, panels and moulded and extruded profiles.

The composite KatG plastics of the invention may be used to produce controlled-release drug delivery systems in which a drug is contained within a composite KatG plastic, for example in the form of a dosage form or device. Examples of suitable forms, devices and drugs are described in WO2019020679.

### 6.9.7 Autodegradation of Composite KatG Plastics

Autodegradation of the composite KatG plastics of the invention may be initiated by contacting the composite KatG plastic with an aqueous reaction mix under conditions where the KatG prodegradant is activated to begin enzymatically degrading the PA polymer with which it is associated.

For example, in preferred embodiments where the KatG prodegradant comprises one or more helper enzymes selected from: (a) formate oxidase; (b) formate dehydrogenase; (c) formaldehyde dismutase; and (d) methanol oxidase (as described above), activation can be achieved by the simple expedient of grinding the composite KatG plastic and contacting the grounds with an aqueous reaction mix comprising methanol and/or ethanol and/or formate.

It will also be appreciated that autodegradation may be activated by exposure to moisture and oxygen in the environment following disposal, particularly where the KatG prodegradant composition comprises appropriate co-reactants (such as methanol and/or ethanol and/or formate), or when the latter is scavenged from the environment by the KatG enzyme.

In embodiments where the PAase enzyme is present in the form of an inactive proenzyme (see Section 6.9.2, above), autodegradation may be activated by exposure to an activating agent (for example, hydrolytic enzymes or acids) which cleaves the proenzyme form to generate active PAase.

### 7. Methods, Processes and Uses

### 7.1 Methods

The invention provides a method for the enzymatic degradation of a PA polymer substrate, the method comprising the step of contacting said PA polymer substrate with a composition comprising a PAase of the invention under conditions whereby C—C bonds in the PA polymer substrate are cleaved, thereby enzymatically degrading the polymer.

The enzymatic degradation need not completely depolymerize the PA polymer substrate, and that the nature and extent of the degradation may be determined, inter alia, by the PAase enzyme selected, the reaction conditions and the chemical nature and/or physical form of the PA polymer substrate.

Thus, the enzymatic degradation by C—C bond cleavage mediated by the PAase of the invention may merely fragment the PA polymer substrate. For example, in the case of PA copolymers which comprise long tracts (or segments) of non-PA polymers, the enzymatic degradation by C—C bond cleavage mediated by the PAase of the invention may yield fragments comprising non-PA polymer segments derived from the copolymer chain.

In preferred embodiments, the PAase enzymatically degrades the PA polymer substrate into: (a) fragments; and/or (b) oligomers; and/or (c) isolated monomers or repeating units; and/or (d) monomer or repeating unit fragments; and/or (e) monomer or repeating unit derivatives.

Alternatively, or in addition, the enzymatic degradation by C—C bond cleavage may oligomerize the PA polymer substrate. In many cases, the enzymatic degradation effected by the PAase of the invention by C—C bond cleavage yields fragments and oligomers of a PA polymer substrate. In yet other cases, the enzymatic degradation effected by the PAase of the invention by C—C bond cleavage yields: (a) fragments; and/or (b) oligomers; and/or (c) isolated monomers or repeating units; and/or (d) monomer or repeating unit fragments; and/or (e) monomer or repeating unit derivatives of a PA polymer substrate.

In preferred embodiments, the enzymatic degradation by C—C bond cleavage effected by the PAase of the invention reduces the molecular weight of the PA polymer substrate, for example by at least 50%. More preferably, the enzymatic degradation by C—C bond cleavage effected by the PAase of the invention achieves a log reduction of at least 1, 2 or 3 of the PA polymer substrate. In cases where the polymer substrate has a relatively high molecular weight, the enzymatic degradation by C—C bond cleavage effected by the PAase of the invention may achieve correspondingly higher log reductions, for example at least 4, 5 or 6 of the PA polymer substrate.

In preferred embodiments, the PA polymer substrate is as described in Sections 4.1-4.4 (above).

In cases where the PA polymer substrate comprises a PA plastic product, the method preferably further comprises one or more biotic or abiotic processing steps, for example as described in Section 5 (above).

In preferred embodiments, the method is carried out at a temperature comprised between 20° C. and 90° C., preferably between 40° C. and 80° C., more preferably between 50° C. and 70° C., more preferably between 60° C. and 70° C.

In preferred embodiments, the method is carried out at a neutral or acid pH, preferably at an acid pH. For example, the method is preferably carried out at a pH between 2 and 7, preferably at a pH between 3 and 6, more preferably at a pH between 3.5 and 5.5, even more preferably at a pH of about 4.5 to 5.

In preferred embodiments, the PAase of the invention is used in an amount up to 5% by weight of the PA polymer substrate, more preferably up to 1%, even more preferably up to 0.1%, and yet more preferably up to 0.05% by weight of the PA polymer substrate. For example, the amount of PAase of the invention may be in a range of 0.001% to 5% by weight of the PA polymer substrate, preferably in the range of 0.001% to 1%, more preferably in the range of 0.001% to 0.1%, and even more preferably in the range of 0.001% to 0.05% by weight of the PA polymer substrate.

In preferred embodiments, the method further comprises agitating the reactants to improve contact between the PAase and PA polymer substrate and so promote adsorption of the enzyme to the substrate. This step may comprise continuous stirring, for example at a rate of between 100 rpm and 5000 rpm. Alternatively, continuous agitation can be achieved by running the method in an airlift reactor (as described in Section 2.4.1, above).

Those skilled in the art will be able to determine an appropriate reaction time by reference to the nature of the PA polymer substrate, the PAase enzyme selected and the extent of degradation required. In preferred embodiments, the enzymatic degradation may be carried out over a reaction time between 5 and 72 hours. Alternatively, the method may be run continuously in circumstances where continuous reactors are employed (see Section 2, above).

The method may further comprise the step of isolating the degradation product(s) produced. Suitable isolation steps include stripping, separation by aqueous solution, steam selective condensation, filtration, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration and combinations of two or more of the foregoing.

### 7.2 Processes for Producing Degradation Products

The invention provides a process for producing: (a) fragments; and/or (b) oligomers; and/or (e) monomer or repeating unit derivatives, of a PA polymer substrate, the process comprising the step of degrading said substrate by contacting it with a composition comprising a PAase of the invention under conditions whereby C—C bonds in the PA polymer substrate are cleaved, thereby enzymatically degrading the polymer.

The enzymatic degradation need not completely depolymerize the PA polymer substrate, and the nature and extent of the degradation may be determined, inter alia, by the PAase enzyme selected, the reaction conditions and the chemical nature and/or physical form of the PA polymer substrate.

Thus, the enzymatic degradation by C—C bond cleavage mediated by the PAase of the invention may merely fragment the PA polymer substrate. For example, in the case of PA copolymers which comprise long tracts (or segments) of non-PA polymers, the enzymatic degradation by C—C bond cleavage mediated by the PAase of the invention may yield fragments comprising non-PA polymer segments derived from the copolymer chain.

In preferred embodiments, the PAase enzymatically degrades the PA polymer substrate into: (a) fragments; and/or (b) oligomers; and/or (c) isolated monomers or repeating units; and/or (d) monomer or repeating unit fragments; and/or (e) monomer or repeating unit derivatives.

Alternatively, or in addition, the enzymatic degradation by C—C bond cleavage may oligomerize the PA polymer substrate. In many cases, the enzymatic degradation effected by the PAase of the invention by C—C bond cleavage yields fragments and oligomers of a PA polymer substrate. In yet other cases, the enzymatic degradation effected by the PAase of the invention by C—C bond cleavage yields: (a) fragments; and/or (b) oligomers and/or (e) monomer or repeating unit derivatives, of a PA polymer substrate. In more detail, the invention provides the generation and use of the products of the reaction of the enzyme with polyalkenes in the presence of O₂ and/or peroxides, such as diacids. Such diacids include oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids. Other oxidised compounds contemplated include glycolic acid, glyoxylic acid, citric acid, furoic acid phenylpyruvic acids

In preferred embodiments, the PA polymer substrate is as described in Sections 4.1-4.4 (above).

In cases where the PA polymer substrate comprises a PA plastic product, the process preferably further comprises one or more biotic or abiotic processing steps, for example as described in Section 5 (above).

In preferred embodiments, the process is carried out at a temperature between 20° C. and 90° C., preferably between 20° C. and 60° C., more preferably between 20° C. and 50° C., more preferably between 20° C. and 40° C.

In preferred embodiments, the process is carried out at a neutral or acid pH, preferably at an acid pH. For example, the method is preferably carried out at a pH between 2 and 7, preferably at a pH between 3 and 6, more preferably at a pH between 3.5 and 5.5, even more preferably at a pH of about 4.5 to 5.

In preferred embodiments, the PAase of the invention is used in an amount up to 5% by weight of the PA polymer substrate, more preferably up to 1%, even more preferably up to 0.1%, and yet more preferably up to 0.05% by weight of the PA polymer substrate. For example, the amount of PAase of the invention may be in a range of 0.001% to 5% by weight of the PA polymer substrate, preferably in the range of 0.001% to 1%, more preferably in the range of 0.001% to 0.1%, and even more preferably in the range of 0.001% to 0.05% by weight of the PA polymer substrate.

In preferred embodiments, the process further comprises agitating the reactants to improve contact between the PAase and PA polymer substrate and so promote adsorption of the enzyme to the substrate. This step may comprise continuous stirring, for example at a rate of between 100 rpm and 5000 rpm. Alternatively, continuous agitation can be achieved by running the process in an airlift reactor (as described in Section 2.4.1, above).

Those skilled in the art will be able to determine an appropriate reaction time by reference to the nature of the PA polymer substrate, the PAase enzyme selected and the extent of degradation required. In preferred embodiments, the enzymatic degradation may be carried out over a reaction time between 5 and 72 hours. Alternatively, the process may be run continuously in circumstances where continuous reactors are employed (see Section 2, above).

The process may further comprise the step of isolating the fragments, oligomers, isolated monomers or repeating units, monomer or repeating unit fragments and/or monomer or repeating unit derivatives.

Suitable isolation steps include stripping, separation by aqueous solution, steam selective condensation, filtration, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration and combinations of two or more of the foregoing.

### 7.3 Uses of the PAases of the Invention

The invention contemplates various uses for the PAase of the invention, including the use of a composition comprising the PAase for the degradation of a PA polymer substrate (for example, any of the PA polymer substrate as described in Section 4, above).

The invention also contemplates the use of a PAase of the invention for producing fragments, oligomers, isolated monomers or repeating units, monomer or repeating unit fragments and/or monomer or repeating unit derivatives of a PA polymer substrate.

The invention also contemplates the use of a PAase of the invention for the bioremediation of material contaminated with a PA polymer substrate.

### 7.4 Processes for Producing Microbial Biomass

The invention provides a process for producing microbial biomass comprising the steps of: (a) providing a seed culture comprising at least one microorganism expressing a KatG/EC 1.11.1.21 PAase; (b) providing a feedstock comprising a PA polymer substrate; (c) mixing said seed culture with said feedstock to form a seeded feedstock; and (d) incubating the seeded feedstock under conditions whereby C—C bonds in the PA polymer substrate are cleaved to produce degradation products of said polymer which are metabolized by said microorganism to yield said microbial biomass and/or other products. In a preferred embodiment the microorganism expressing the KatG/EC 1.11.1.21 PAase obtains its carbon source from the degradation of polyalkene polymers

The microbial biomass may be recovered in any suitable form (e.g. pelletized by centrifugation). It may be subsequently processed (e.g. lysed and/or fractionated). It may subsequently be used as starting material for the isolation of bioproducts, including as a source of protein and/or amino acids, for example for animal feeds.

Other products of the reaction of the KatG enzyme and its derivatives with polyalkenes may also be recovered from both the pellet and the supernatant according to established methods.

### 7.5 Processes for Producing Bioproducts

The invention provides a process for producing a bioproduct comprising the steps of: (a) providing a seed culture comprising at least one microorganism expressing a KatG/EC 1.11.1.21 PAase; (b) providing a feedstock comprising a PA polymer substrate; (c) mixing said seed culture with said feedstock to form a seeded feedstock; and (d) incubating the seeded feedstock under conditions whereby C—C bonds in the PA polymer substrate are cleaved by said enzyme to produce degradation products of said polymer which are metabolized by said microorganism to yield said bioproduct.

The bioproduct may comprise a biofuel, for example a biofuel selected from bioalcohol (e.g. bioethanol), biodiesel, biogas and syngas. Alternatively, or in addition, the bioproduct may comprises ethylene glycol and/or propylene glycol.

The bioproduct may comprise one or more dicarboxylic acids and/or one or more aldehydes. The bioproduct may comprise one or more dicarboxylic acids. The bioproduct may comprise one or more aldehydes. The carbon chain may be saturated, partially saturated or unsaturated, branched or straight chain.

The dicarboxylic acid (also known as a “diacid”) can be selected from any compound with the formula CₙFₙ₋₂O₄, wherein n can be selected from 2 to 20 The value of n can be selected from 2, 3, 4, 5, 6, 7, 8, 9, 10, 11, 12, 13, 14,15,16,17,18, 19 or 20. Preferably, the value of n can be 3 (i.e. Malinoic acid). The products of the reaction of the enzyme with polyalkenes in the presence of O₂ and/or peroxides, such as diacids, such as diacids include oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids. Other oxidised compounds contemplated include glycolic acid, glyoxylic acid, citric acid, furoic acid phenylpyruvic acids.

Such compounds have high value as specialty chemicals, and involved in a number of manufacturing processes such as in the electronics, flavour and fragrance, specialty solvents, polyesters, polymer cross-linking, food additives and pharmaceutical industries. By way of further example, malonic acid (and malonate; MA) is used as a building block chemical to produce diverse valuable compounds. Malonic acid and chemical derivatives of malonic acid (such as, for example, monoalkyl malonate, dialkyl malonate, and 2,2-dimethyl-1,3-dioxane-4,6-dione (“Meldrum's acid”)) are used for the production of many industrial and consumer products, including polyesters, protective coatings, solvents, electronic products, flavors, fragrances, pharmaceuticals, surgical adhesives, and food additives.

The aldehyde can be selected from any compound with the formula CₙFₙ₋₂O₃, wherein n can be selected from 2 to 20.

### 7.6 Processes for Producing a Hyperactive KatG Enzyme

The invention provides a process for producing a hyperactive KatG enzyme comprising the steps of: (a) providing a wild-type KatG enzyme having a first PAase activity; (b) providing a plurality of mutant forms of said wild-type KatG enzyme which has one or more amino acid substitutions, deletions or insertions relative to the amino acid sequence of the wild-type KatG; (c) determining the PAase activity of the mutant forms of step (b); and (d) identifying a hyperactive KatG enzyme having a PAase activity which is higher than said first PAase activity.

Preferably, the wild-type KatG enzyme of step (a) is B2UBU5. The mutant form of step (b) may be provided by site-directed mutagenesis or directed evolution. Step (c) preferably comprises the step of contacting a PA polymer with the mutant forms of step (b) and monitoring the degradation of the polymer by a method comprising one or more analytical methods selected from: chemical, electrochemical, morphological, rheological, gravimetric, spectroscopic and/or chromatographic methods.

In preferred embodiments, step (c) comprises contacting a suspension of microbeads formed from the PA polymer with the mutant forms of step (b) and then monitoring the decrease in the optical density of the microbead suspension attendant on microbead degradation.

In preferred embodiments, step (c) comprises contacting the mutant forms of step (b) with microbeads releasably coupled to a chromophore or fluorophore, which chromophore or fluorophore is released by PAase activity. In such embodiments, the microbeads may be releasably coupled to a chromophore or fluorophore via a polyethylene linker which is susceptible to cleavage by PAase activity.

Alternatively, the microbeads may comprise a PA polymer and the chromophore or fluorophore is incorporated or encapsulated within the microbead and released when the microbead is exposed to PEase activity.

In preferred embodiments, step (d) comprises flow cytometry whereby the hyperactive KatG enzyme is identified by high-throughput screening.

The process preferably further comprises the step of determining the amino acid sequence of the hyperactive KatG enzyme. In such embodiments, the process may further comprise the steps of synthesising DNA encoding said amino acid sequence and then expressing said encoding DNA in a cellular expression system, for example an expression system as defined and described above.

### 8. KatG Assay Kits

The invention provides an assay kit for the high-throughput screening of KatG mutant forms comprising a microbead releasably coupled to a chromophore or fluorophore, which chromophore or fluorophore is released by PAase activity. The microbeads may comprise a PA polymer and the chromophore or fluorophore may be incorporated or encapsulated within the microbead and released when the microbead is exposed to PAase activity.

In another aspect, the invention provides an assay kit for the high-throughput screening of KatG mutant forms comprising PA polymer microbeads which can be suspended in a reaction medium and degraded by PAase activity. The assay kit of the invention may further comprise a KatG enzyme, for example B2UBU5.

### EXEMPLIFICATION

The invention will now be described with reference to specific Examples. These are merely exemplary and for illustrative purposes only: they are not intended to be limiting in any way to the scope of the monopoly claimed or to the invention described.

### FIGURE LEGENDS

FIG. 1: Mass loss of polyethylene (grey) and polypropylene (black) for 100 mg (panel A) and 200 mg (panel B) incubations with Ralstonia pickettii (ATCC 27511) over a 28 day period.

FIG. 2: Biodegradation of LDPE film after incubation with Ralstonia pickettii (ATCC 27511).

FIG. 3: Effect of Ralstonia pickettii culture supernatant on the biodegradation of polyethylene beads.

FIG. 4: Biodegradation of low-density polyethylene (LDPE) films with Ralstonia pickettii supernatant.

FIG. 5: Biodegradation of polyethylene beads by a microbial extract containing recombinant Ralstonia pickettii KatG

FIG. 6: Biodegradation of polyethylene beads by purified recombinant His-tagged KatG

FIG. 7: High-throughput flow cytometric assay of degradation of fluorescent PS bead by recombinant KatG

FIG. 8: Plastic biodegradation using KatG-helper enzyme system

FIG. 9: Production of malonic acid from various polymers incubated with KatG and H₂O₂.

FIG. 10: Production of various compounds from polyethylene incubated with KatG and H₂O₂.

FIG. 11: Production of malonic acid from polyethylene incubated with KatG but without H₂O₂.

FIG. 12: Production of various compounds from polyethylene incubated with KatG but without H₂O₂.

FIG. 13: Mass spectrum of malonic acid as produced by incubation of polyethylene with KatG, and the mass spectrum of an authentic standard.

FIG. 14: Mass spectrum of sebacic acid as produced by incubation of polyethylene with KatG, and the mass spectrum of an authentic standard.

FIG. 15: Mass spectrum of azelaic acid as produced by incubation of polyethylene with KatG, and the mass spectrum of an authentic standard.

### EXAMPLE 1: GROWTH OF RALSTONIA PICKETTII (ATCC 27511) ON PLASTIC FILMS

Liquid Carbon-Free Basal Medium (LCFBM) is a medium lacking any added carbon source and is described by Yang et al. (2014) Evidence of polyethylene biodegradation by bacterial strains from the guts of plastic-eating waxworms Environ Sci Technol 48: 13776-13784). Parallel 50 mL LCFBM cultures of R. picketti were set up with either 100 mg or 200 mg of sterilised PP or PE powders (Goonvean Fibres HM20/70P and Goodfellow ET316031). Cultures were inoculated with an existing stock of R. pickettii, and incubated at 30° C. for 3, 7, 10, 14, 21 and 28 days. Upon removal, each culture was spun down to remove the cells. The plastic was poured off, strained, and washed in 2% SDS solution for 4 h. The plastics were then washed in distilled water 4 times and left to dry overnight. They were then weighed to record gravimetric change.

Controls were prepared with polymers but without cells, using the same washing and drying process as all other cultures. No growth was observed nor polymer degradation.

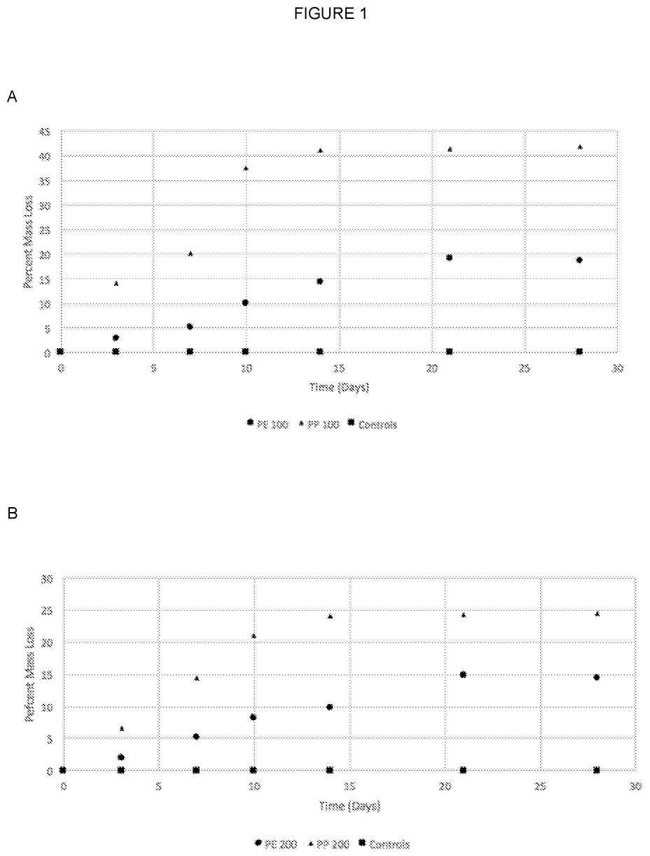

FIG. 1 shows the % mass loss of polyethylene (grey) and polypropylene (black) for the 100 mg (A) and 200 mg (B) incubations.

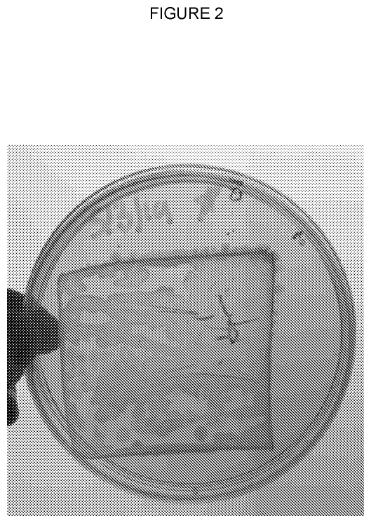

LDPE film (Goodfellow ET311126) was cut into 50 mm×50 mm squares, disinfected in 70% ethanol, and dried overnight in a sterile hood. LCFBM agar was prepared (Yang et al. (2014) op. cit.) and 50 μL of a Ralstonia picketii culture was pipetted into the middle of the plate, before being spread by a sterile loop. An LDPE square was then placed over the inoculum, and the plate was sealed with Parafilm™ before incubation for 3 days at 30° C. Large areas of biodegradation are clearly observable (FIG. 2).

### EXAMPLE 2: BIODEGRADATION OF PE BEADS WITH RALSTONIA PICKETTII CULTURE SUPERNATANT

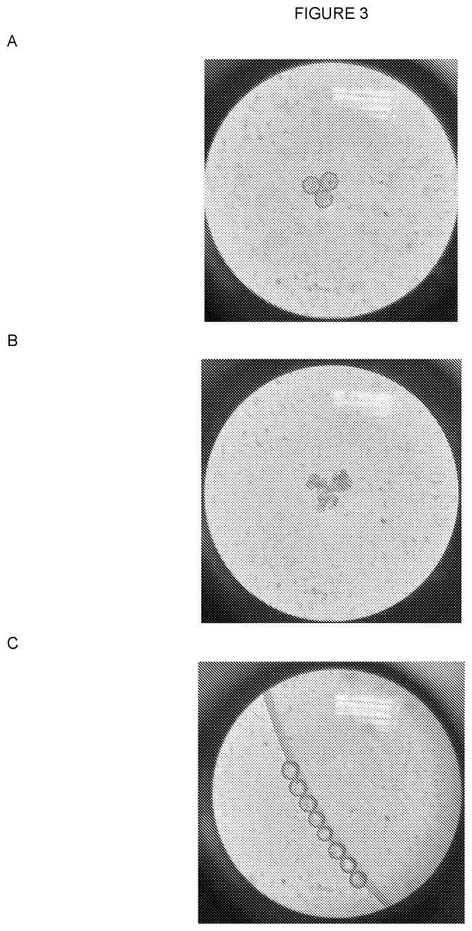

2.24 mg of 32-38 μm Cospheric PE Microspheres (UVPMS-BY2-1.00) were placed in a glass vial, and suspended in 1 mL of LCFBM (as above), and incubated for 2 days at 30° C. with 5 mM H₂O₂. Samples were vortexed and pipetted onto a glass slide before visualization under a benchtop microscope. The beads were unchanged (FIG. 3A)

R. pickettii cultures were grown with sterilised polypropylene at a concentration of 100 mg per 100 mL (Goonvean Fibres HM20/70P) in LCFBM (as above) for 3 days at 30° C. Cultures were centrifuged for 3 minutes at 10,000×g. The supernatant was removed, and was concentrated using an Amicon Stirred Cell Concentrator. 1 mL of 120-fold concentrated supernatant was added to 2.24 mg of 32-38 μm Cospheric microspheres (UVPMS-BY2-1.00 32-38 μm) together with 5 mM H₂O₂ and incubated for 2 days at 30° C. Samples were vortexed and pipetted onto a glass slide before visualization under a benchtop microscope. Much degradation had evidently occurred (FIG. 3B)

R. pickettii cultures were grown, and the supernatant obtained and concentrated, precisely as described above, save that before adding to the Cospheric microspheres the supernatant was boiled first for 10 min. The beads were unchanged (FIG. 3C).

### EXAMPLE 3: BIODEGRADATION OF LOW-DENSITY POLYETHYLENE (LDPE) FILMS WITH RALSTONIA PICKETTII SUPERNATANT

100 mg of LDPE film (Goodfellow ET311126) was sterilised by incubation in 0.1M Sodium Hydroxide for 1 hour followed by 5× washes with sterile water and 1 wash with LCFBM before drying in a sterile hood. The films were added to 50 mL of LCFBM, and incubated for 3 days. The films were imaged using a Keyence VHX-7000 microscope.

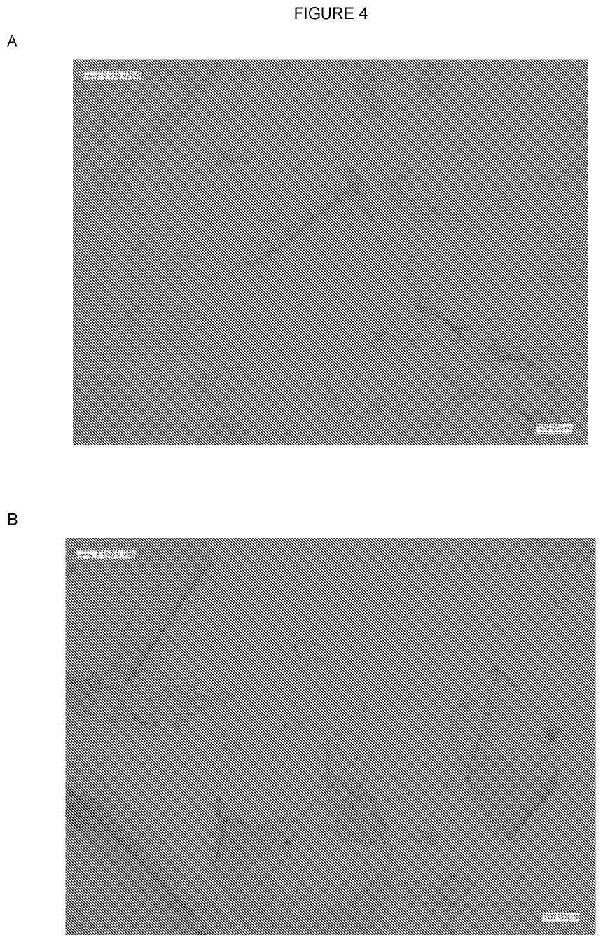

The films remained intact (FIG. 4A).

100 mg of LDPE film (Goodfellow ET311126) was sterilised as described above. The films were added to 50 mL of R. pickettii supernatant prepared as described in Example 2 and incubated for 3 days. The films were imaged using a Keyence VHX-7000 microscope.

Extensive fragmentation of the polymer film was observed (FIG. 4B).

### EXAMPLE 4: BIODEGRADATION OF POLYETHYLENE BEADS BY A MICROBIAL EXTRACT CONTAINING RECOMBINANT RALSTONIA PICKETTII KATG

KatG from R. picketii was cloned into a pET vector, E. coli BL21 cells were transformed to incorporate this plasmid. Lysates were prepared by growing 50 ml of culture at 30° C. in Auto Induction Terrific Broth (ForMedium) and resuspending the centrifuged bacterial pellet in 2 ml Acetate buffer (sodium acetate 45 mM, Acetic acid 55 mM, 50 mM sodium chloride, pH 4.5) supplemented with 0.1 mg/ml lysozyme. Samples were subjected to bead beating at 29.9 KHz for 2 min with approx. 30 mg of 0.5 mm Zirconia beads in a Tissue Lyser II (Qiagen) and subsequent incubation at 37° C. for 1h with shaking at 500 rpm. Cell free lysates were obtained by centrifugation of cell debris at 10,000 g for 10 min.

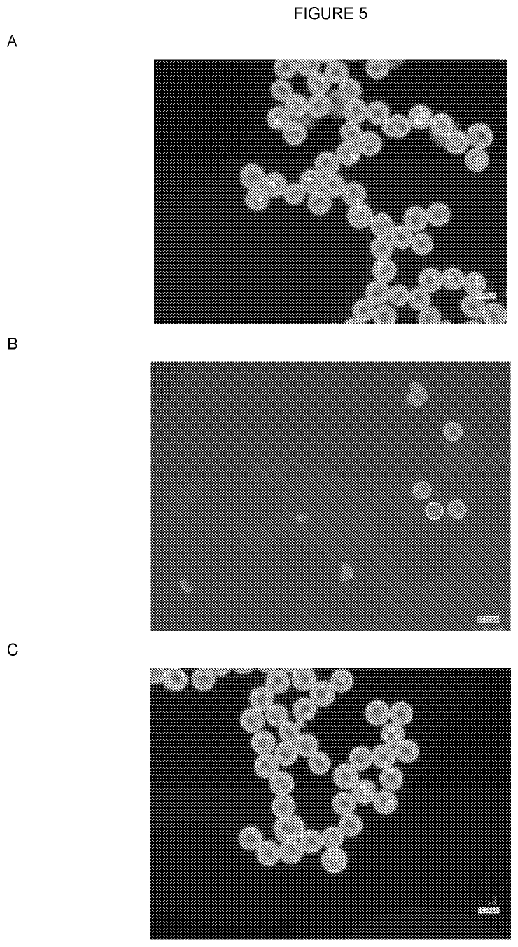

2.24 mg of 32-38 μm Cospheric Microspheres (UVPMS-BY2-1.00 32-38 μm) were placed in a glass vial, and suspended in 200 μm of LCFBM (as above). H₂O₂ was added to a final concentration of 5 mM, and the sample was incubated for 2 days at 30° C. Samples were vortexed and visualised under a Keyence VHX-7000 microscope. No degradation was observed (FIG. 5A).

2.24 mg of 32-38 μm Cospheric Microspheres (UVPMS-BY2-1.00 32-38 μm) were placed in a glass vial. 200 uL of recombinant KatG lysate was extracted from cells, and added to the beads. H₂O₂ was added to a final concentration of 5 mM. The sample was incubated for 2 days at 30° C. Samples were vortexed and visualised under a Keyence VHX-7000 microscope. Considerable degradation was observed (FIG. 5B).

2.24 mg of 32-38 um Cospheric Microspheres (UVPMS-BY2-1.00 32-38 um) were placed in a glass vial. 200 uL of recombinant KatG lysate was extracted from cells, and boiled for 10 mins at 99° C. The lysate was cooled to room temperature, and added to the beads. H₂O₂ was added to a final concentration of 5 mM. The sample was incubated for 2 days at 30° C. Samples were vortexed and visualised under a Keyence VHX-7000 microscope. No degradation was observed (FIG. 5C).

### EXAMPLE 5: BIODEGRADATION OF POLYETHYLENE BEADS BY PURIFIED RECOMBINANT HIS-TAGGED KATG

2.24 mg of 32-38 μm Cospheric Microspheres (UVPMS-BY2-1.00 32-38 μm) were placed in a glass vial, and suspended in 200 μm of LCFBM (as above). H₂O₂ was added to a final concentration of 5 mM, and the sample was incubated for 2 days at 30° C. Samples were vortexed and visualised under a Keyence VHX-7000 microscope. No degradation was observed (FIG. 6A).

2.24 mg of 32-38 μm Cospheric Microspheres (UVPMS-BY2-1.00 32-38 μm) were placed in a glass vial. 2.24 mg of 32-38 μm Cospheric Microspheres (UVPMS-BY2-1.00 32-38 μm) were placed in a glass vial. 10 mL of recombinant KatG lysate was extracted from cells using B-Per lysis buffer (Thermo) and purified using His-Pur Ni-NTA Magnetic Beads (Thermo) according to manufacturer's instructions. 200 μL of purified enzyme solution was added to the beads, and H₂O₂ was added to a final concentration of 5 mM. The sample was incubated for 2 days at 30° C. Samples were vortexed and visualised under a Keyence VHX-7000 microscope. Considerable degradation was observed (FIG. 6B).

### EXAMPLE 6: BIODEGRADATION OF POLYPROPYLENE AND POLYETHYLENE BEADS BY PURIFIED RECOMBINANT HIS-TAGGED KATG AND KATG VARIANTS

Mass loss attendant on incubation with recombinant KatG and derivatives was evaluated using polypropylene (PP; Goonvean Fibres HM20/70P) and polyethylene (PE; Cospherics CPMS-0.96 38-45 μm) beads.

Pre-weighed 1.5 ml Eppendorf centrifugation tubes were supplemented with PP or PE beads and the tube weight rechecked to determine initial bead weight. 256-3 and 256-6 are BL21 E. coli lysates expressing recombinant R. pickettii KatG enzyme. 256-3 is the wild-type sequence, while 256-6 is a Y221F/R410N variant of the wild-type sequence. All recombinant KatG enzymes tested included a C-terminal His tag.

Lysates were prepared by growing 50 ml of culture at 37° C. in Auto Induction Terrific Broth (ForMedium) and resuspending the centrifuged bacterial pellet in 2 ml acetate buffer (sodium acetate 45 mM, Acetic acid 55 mM, 50 mM sodium chloride, pH 4.5) supplemented with 0.1 mg/ml lysozyme. Samples were subjected to bead beating at 29.9 kHz for 2 min with approx. 30 mg of 0.5 mm Zirconia beads in a Tissue Lyser II (Qiagen) and subsequent incubation at 37° C. for 1h with shaking at 500 rpm. Cell-free lysates were obtained by centrifugation of cell debris at 10,000 g for 10 min at 4° C.

A type strain of R. pickettii (ATCC 275111) was used to inoculate 500 ml of Liquid Carbon Free Basal Medium (see above) with 20 g of Cling film as carbon source and incubated at 30° C. for 5 days on a shaker. The supernatant was recovered by centrifugation of the cell pellet and concentration via an Amicon stirred cell concentrator and buffer exchange into the above acetate buffer to a final volume of approximately 2 ml.

For assay 400 μl of lysate/supernatant supplemented with H₂O₂ (5 mM final concentration) was vortexed with the beads and incubated at 30° C. for 24h at 200 RPM. Aqueous solution was separated from beads by centrifugation at 8000 g for 5 min and beads subsequently washed with water then ethanol with separation again being achieved by centrifugation. Residual ethanol was removed by vacuum centrifugation (SpeedVac, Thermo). The weight of the dry beads and tube was again checked to determine mass loss.

Table 1 (below) shows the mass loss after just 24h of incubation. Notable mass loss was observed for both polypropylene and polyethylene beads relative to incubation in buffer/H₂O₂ only.

TABLE 1

| | | start | end | Mass | | |
|---|---|---|---|---|---|---|
| | Bead | weight | weight | change | % | average |
| Sample ID | type | (mg) | (mg) | (mg) | loss | % |
| 256-3 | PP | 41.97 | 39.91 | −2.06 | 4.91 | 6.47 |
| 256-3 | PP | 20.92 | 19.24 | −1.68 | 8.03 | |
| 256-6 | PP | 17.91 | 15.77 | −2.14 | 11.95 | 11.62 |
| 256-6 | PP | 30.21 | 26.8 | −3.41 | 11.29 | |
| R. Pickettii SN | PP | 20.29 | 15.36 | −4.93 | 24.30 | 17.41 |
| R. Pickettii SN | PP | 30.25 | 27.07 | −3.18 | 10.51 | |
| Acetate Buffer | PP | 12.87 | 11.64 | −1.23 | 9.56 | 5.35 |
| Acetate Buffer | PP | 17.47 | 17.27 | −0.20 | 1.14 | |
| 256-3 | PS | 19.6 | 19.17 | −0.43 | 2.19 | 3.35 |
| 256-3 | PS | 12.42 | 11.86 | −0.56 | 4.51 | |
| 256-6 | PS | 20.36 | 19.36 | −1.00 | 4.91 | 6.21 |
| 256-6 | PS | 10.51 | 9.72 | −0.79 | 7.52 | |
| R. Pickettii SN | PS | 11.14 | 11.03 | −0.11 | 0.99 | 1.81 |
| R. Pickettii SN | PS | 15.97 | 15.55 | −0.42 | 2.63 | |
| Acetate Buffer | PS | 17.4 | 16.94 | −0.46 | 2.64 | 1.64 |
| Acetate Buffer | PS | 9.52 | 9.46 | −0.06 | 0.63 | |

### EXAMPLE 7: HIGH-THROUGHPUT FLOW CYTOMETRIC ASSAY OF DEGRADATION OF FLUORESCENT PE BEAD BY RECOMBINANT KATG

Cell extract 256-3 was prepared as in Example 6. 200 μl was then used to desalt into PBS adjusted to pH 5 via a Zeba spin desalt column, to which 1.2 mg of Cospherics beads as described in Example 6 were added. The fluorescent beads consist of polymer spheres through which are dispersed individual ‘rocks’ of a yellow, water-insoluble fluorophore. 150 ul was used in the above assay.

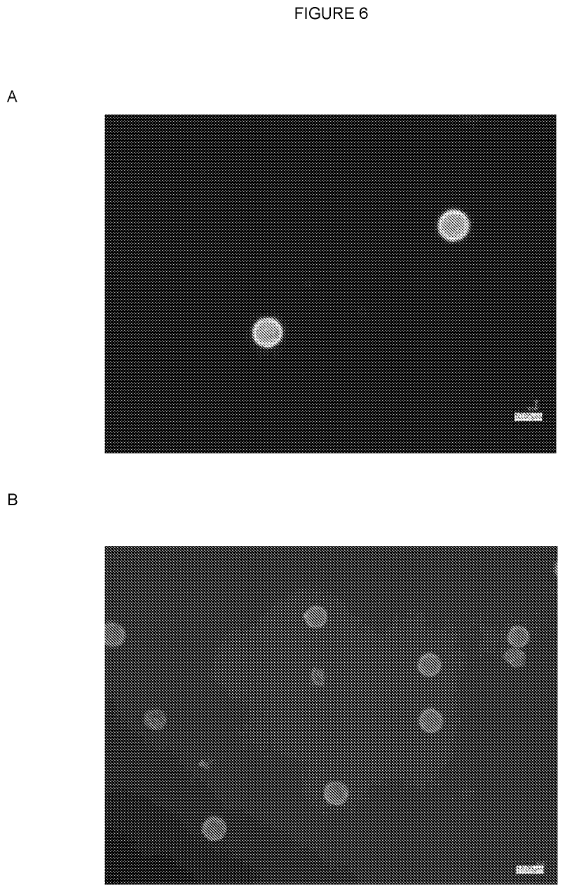

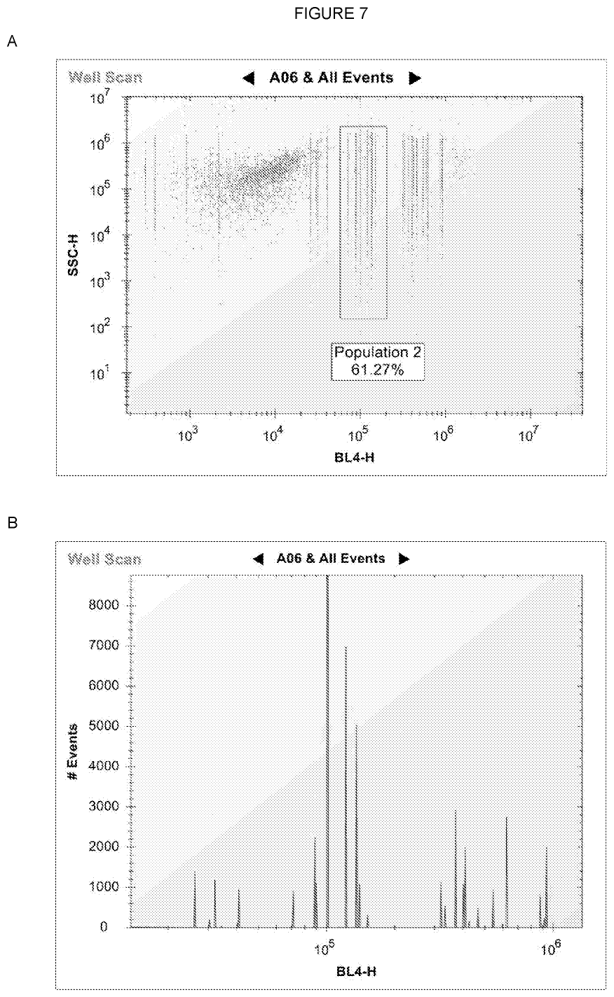

Samples were analysed using an Intellicyt iQue Screener Plus; the data shown used excitation at 488 nm and emission at 675±30 nm. FIG. 7A shows a dot plot of side scatter (indicating size) vs fluorescence of particles detected (the BL4 detection channel on this instrument). The initial size of the beads means that their side scatter is actually off the scale (>10⁷ units). The discretised size of the fluorescence signal reflects the discrete nature of the fluorescent inclusions in the beads, as well as plastic-free fluorophore particles. Many of the events between 2,000 and 20,000 are background noise. It is very obvious that extremely extensive degradation of the beads has occurred, as observed in both the light scattering and fluorescence channels. FIG. 7B shows a subset of the data of A as a histogram of fluorescence, emphasising its discrete nature.

### EXAMPLE 8: PLASTIC BIODEGRADATION USING KATG-HELPER ENZYME SYSTEM

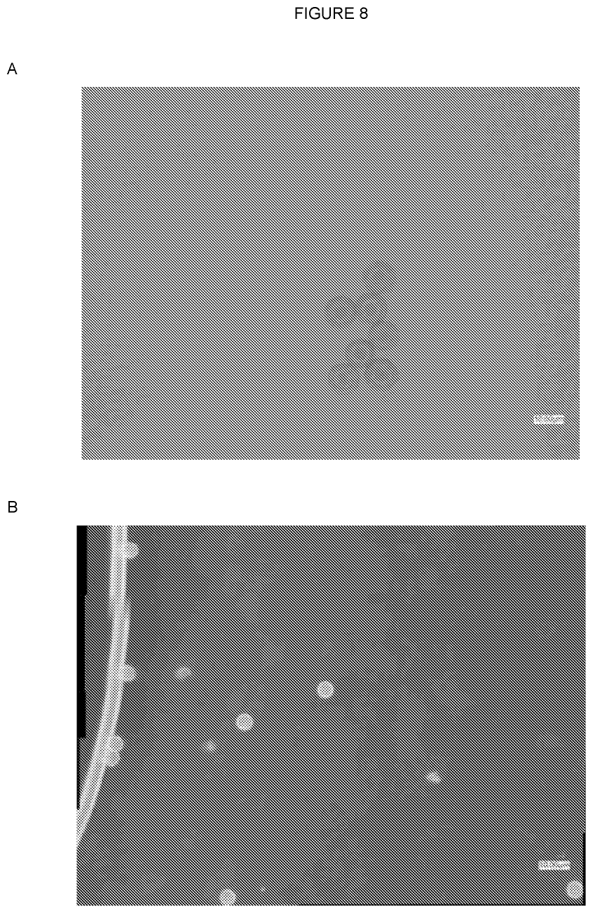

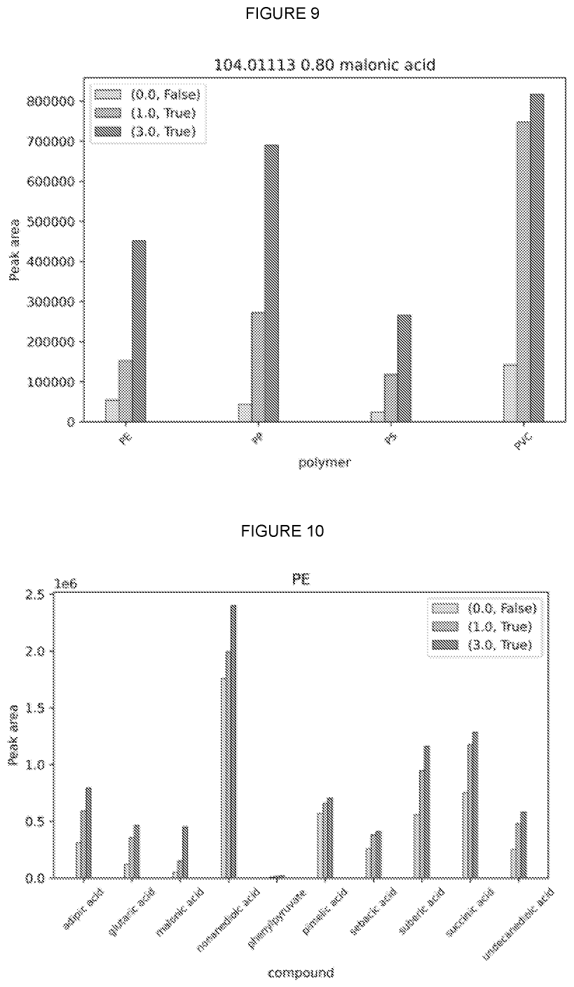

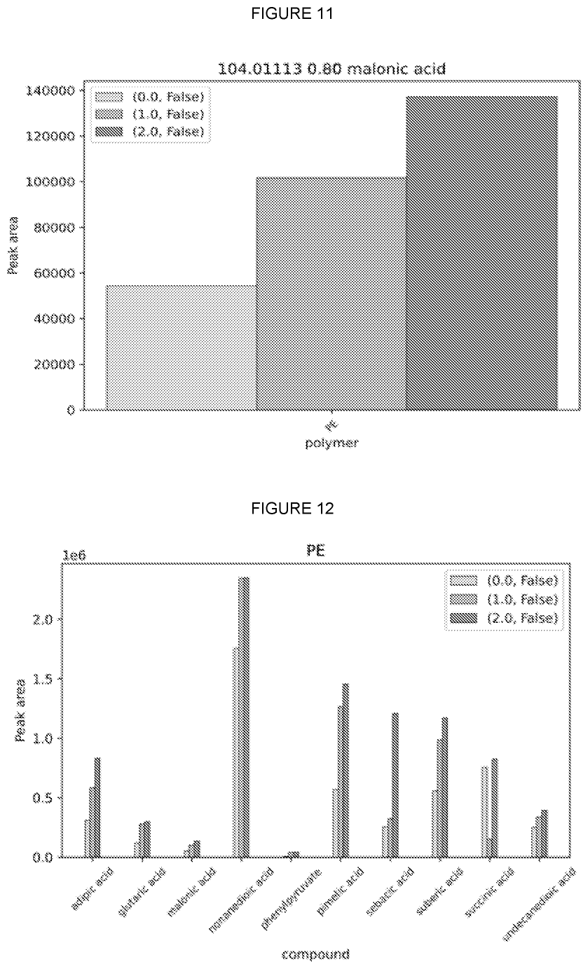

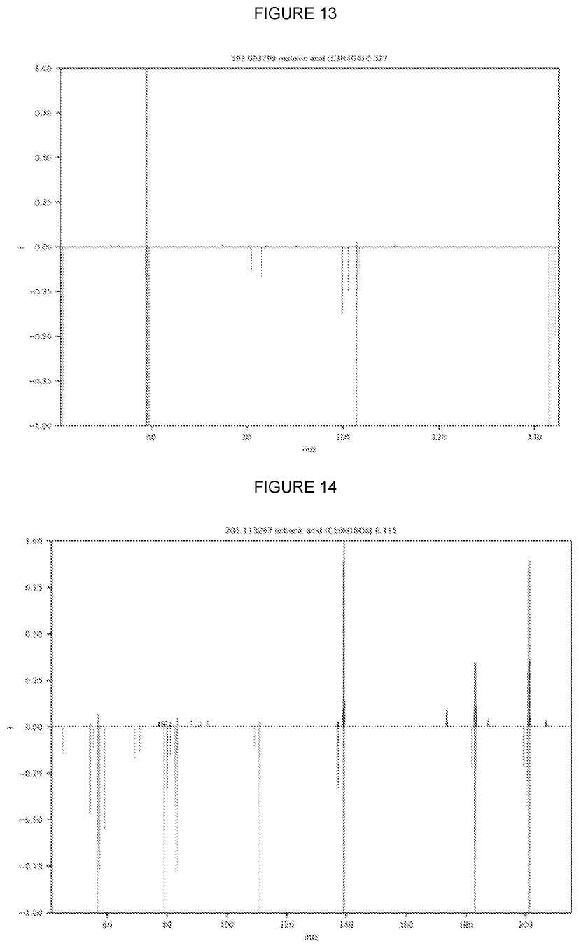

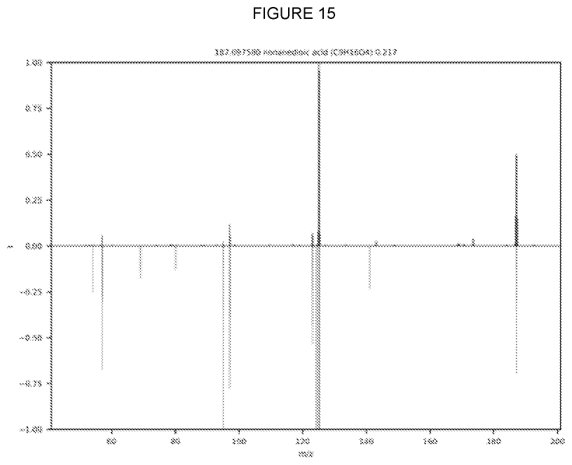

An H₂O₂-regenerating system was employed using glucose (D+) and glucose oxidase from Aspergillus niger (both from Sigma). Assays were supplemented with 0.1M glucose and 2U glucose oxidase, but were otherwise performed with cell extract 256-3 as in Example 7 for a period of 4 days. FIG. 8A shows the control without enzyme, while FIG. 8B shows the degradative effect of the enzyme system.

### EXAMPLE 9: PRODUCTION OF CHEMICALS BY PURIFIED KATG INCUBATED WITH POLYALKENE PLASTICS, WITH AND WITHOUT H₂O₂

### Lysate

Version 256-6. A Y221F/R410N katG mutant was transformed into BL21 E. coli and selected colonies were grown on in LB including carbenicillin. The LB culture was used to inoculate 1 L of Terrific Broth Auto Induction and grown for 24h at 30 C. The resulting bacterial growth was recovered by resuspension in 10 ml of 50 mM Sodium phosphate dibasic and 0.1 mg/ml Lysosyme and 100 mg/ml DNAse I and incubated with shaking for 1h at 30 C. The suspension was subject to cell lysis via a Constant Cell Disruption System (Constant Systems) and the disrupted lysate subject to centrifugation at 12000 g for 15 min. The resulting supernatant of the lysate was recovered and used in subsequent assays. The pH of this lysate was 6.4.

### Purified KatG

Sumo Y221F/R410N katG is a His Tagged protein with a C-terminal peptide sequence recognized by a His-tagged Sumo protease to leave a katG enzyme with traceless tags after digestion. The coding sequence for katG mutant (Y221F/R410N) with an N-terminal cleavable his-SUMO tag was cloned into PE-SUMOPro (LifeSensors, PE-1000-K100) vector with KANAMYCIN resistance marker and transformed into BL21 (DE3) E. coli cells. The clone was prepared as described for 256-6 (as in Example 6) with the exception that the bacterial cells were resuspended in 20 mM sodium phosphate buffer pH 7.4, 300 mM NaCl to allow direct loading of the lysate supernatant onto a His Trap™ HP purification column (Cytivia) initialised in same buffer using an Äkta Start Chromatography system with fraction collector. The recombinant enzyme was purified by gradient elution with 20 mM sodium phosphate buffer pH 7.4, 300 mM NaCl, 500 mM imidazole. The fractionated His-tagged KatG enzyme (approx. 12 ml) was then subject to incubation with 200 ml of 7 mg/ml Sumo protease overnight at 25° C. with dialysis using Snakeskin® dialysis tubing (Thermo Scientific) against 50 mM Tris-HCl, pH 8.0/150 mM NaCl/1 mM DTT. The dialysed, Sumo protease-digested KatG was then returned to the His Trap column and the tag-free mutant (Y221F/R410N) collected by flow through off column with the His tags retained on the column until gradient elution with imidazole.

The purified KatG mutant (Y221F/R410N) in 20 mM sodium phosphate buffer pH 7.4, 300 mM NaCl was then subjected to buffer exchange using PD-10 desalting columns into 0.1M sodium acetate pH 5 and used in subsequent assays. In this example, this preparation is used in the experiments below, and referred to as KatG.

### Incubation Conditions

Ultra high molecular weight polyethylene (UHMWPE) powder (Sigma 434272), 14-20 μm diameter polystyrene microspheres (Cospheric PSMS-1.07), polyvinyl chloride (PVC) powder (Goodfellow 996-673-33), polypropylene (PP) powder (Goonvean Fibres HM20/70P) and Polytetrafluoroethylene (PTFE) powder (Goodfellow 531-672-48) were weighed into separate glass vials. Purified KatG mutant (Y221F/R410N) enzyme and 0.1 M acetate buffer pH5, were added to vials to give final enzyme and polymer concentrations of 1.25 μM (^{˜}0.1 mg·mL⁻¹) and 5 mg/mL, respectively, in a total reaction volume of 2.5 mL. For each polymer two biological replicate reactions were set up alongside controls using purified KatG denatured by incubating with 10 mM DTT (REF) at 80° C. for 30 minutes and an enzyme negative control. All samples were incubated at 30° C. and 250 RPM. Every hour samples were removed and dosed with 2 μL 9.8 M hydrogen peroxide (Sigma H1009) diluted 1000× into the reaction mixture.

Aliquots were taken, quenched and stored at −80° C. for analysis by mass spectrometry (MS) prior to the first dose with hydrogen peroxide and incubation. For all samples excluding PTFE, aliquots were then taken after 5 and 10 hours of incubation. Only one aliquot was taken from the PTFE reaction after 5 hours of incubation. Aliquots were added to MS grade methanol pre-cooled to −80° C. Samples in methanol were then vortexed and centrifuged for 15 minutes at 4° C. and 14,000 RPM before transferring supernatants to microfuge tubes which were stored at −80° C. Specifically, the sample is diluted 1 in 4.5 (100 mL sample+350 mL methanol). Then 75 mL of that mix is dried and resuspended in 40 mL prior to injection.

This process was then repeated for UHMWPE without the addition of hydrogen peroxide. Aliquots were taken and quenched for MS as described above after 5 hours and 57 hours of incubation.

Mass Spectrometry

Mass spectra of samples incubated as above were obtained in both positive and negative mode on an Orbitrap instrument using the methods described and published in Open Access form in Wright Muelas M, Roberts I, Mughal F, O'Hagan S, Day P J, Kell D B: An untargeted metabolomics strategy to measure differences in metabolite uptake and excretion by mammalian cell lines. Metabolomics 2020; 16:107. Those displayed here were obtained in using negative ionisation.

### Proton NMR Spectroscopy

¹H 1D NMR spectra were acquired on a Bruker 700 MHz Avance IIIHD spectrometer equipped with a TCI cryoprobe, using standard vendor-supplied pulse sequences (noesygppr1d and cpmgpr1d) acquired with 4s interscan delay 32 transients and 64k points at 298K (25° C.). Temperature calibrated using 99.8% deuterated methanol standard and 3D shimming on standard 2 mM Sucrose reference for quality assurance. Sample quality control for all spectra was performed on the linewidth of acetic acid.

Compounds identified in various extracts, that typically differed in molecular weight by 14, 28 or 56 amu, included oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids. On occasion, glycolic acid, glyoxylic acid, citric acid, furoic and phenylpyruvic acids were also observed.

Malonic and citric acids were specifically confirmed by NMR spectroscopy.

### EQUIVALENTS

The foregoing description details presently preferred embodiments of the present invention. Numerous equivalents, modifications and variations in practice thereof are expected to occur to those skilled in the art upon consideration of these descriptions. Those modifications and variations are, or are intended to be, encompassed by the following claims.

1. A composition suitable for use in the degradation of synthetic plastic polyalkene polymers, said composition comprising a KatG/EC 1.11.1.21 polyalkenase (PAase) enzyme and a suitable excipient.

2. A composition as claimed in claim 1, wherein the KatG/EC 1.11.1.21 PAase is a KatG enzyme.

3. A composition as claimed in claim 1 or 2, wherein the KatG enzyme comprises, consists of, or consists essentially of, the amino acid sequence of B2UBU5 KatG^{N}, or a variant thereof which comprises, consists of, or consists essentially of, an amino acid sequence which is at least 50%, 55%, 60%, 65%, 70%, 75%, 80%, 85%, 90%, 95% or 99% identical to B2UBU5 KatG^{N}.

4. A composition as claimed in 1 or 2, wherein the KatG enzyme comprises, consists of, or consists essentially of, the amino acid sequence of B2UBU5, or a variant thereof which comprises, consists of, or consists essentially of, an amino acid sequence which is at least 50%, 55%, 60%, 65%, 70%, 75%, 80%, 85%, 90%, 95% or 99% identical to B2UBU5.

5. A composition as claims 1 to 4, wherein the KatG enzyme is a B2UBU5 orthologue.

6. A composition as claimed in claim 5, wherein the B2UBU5 orthologue is selected from:
A0A115SK77 (A0A115SK77_9 RALS)
A0A0F0E7J1 (A0A0F0E7J1_9 BURK)
A0A0J9DTW2 (A0A0J9DTW2_9 RALS)
S9RSJ5 (S9RSJ5_9 RALS)
A0A191ZZS8 (A0A191ZZS8_9 RALS)
A0A5C6Y6C3 (A0A5C6Y6C3-9RALS)
C6BD71 (C6BD71_RALP1)
A0A2P4RN88 (A0A2P4RN88_RALPI)
U3GIS7 (U3GIS7_9 RALS)
A0A1COXEH7 (A0A1COXEH7_RALPI)
R0CJV6 (R0CJV6_RALPI)
A0A2N4TPC1 (A0A2N4TPC1_RALPI)
A0A2N5LD46 (A0A2N5LD46_9RALS)
A0A291E192 (A0A291E192_RALPI)
A0A401K9C7 (A0A401K9C7-9RALS)
UPI00046A45B6
UPI000DD31867
UPI000CEF361E
UPI00073F3865
UPI00046AAE20
UPI0012AE63A9
UPI000D5F3903
UPI0004803C34
UPI000CEDCA44

7. A composition as claimed in any one of claims 1 to 6, wherein the KatG enzyme is a hyperactive KatG enzyme.

8. A composition, of any one of claims 1 to 7, wherein the KatG enzyme is a non-catalatic KatG variant.

9. A composition, of claim 8, wherein the non-catalatic KatG variant comprises a disrupted tripeptide adduct.

10. A composition of either claim 8 or 9, wherein the non-catalatic KatG variant is a non-catalatic B2UBU5 variant or a non-catalatic variant of any one of the orthologues of claim 6.

11. A composition, of claims 8 to 10, wherein the non-catalatic KatG variant comprises one or more of the following amino acid substitutions: M2481, M248V, Y221F, Y221A, Y221L, W98A, W98F and/or R410N.

12. A composition as claimed in any of claims 1 to 11, wherein the KatG enzyme is a single domain KatG enzyme.

13. A composition as claimed in claims 1 to 12 further comprising an enzyme capable of generating a peroxide.

14. A composition as claimed in claim 1 or 13 further comprising a plastic binding agent.

15. A composition as claimed in claim 13, wherein the hydrogen peroxide generating enzyme is selected from the group; formate oxidase, formate dehydrogenase, formaldehyde dismutase, methanol oxidase or combinations thereof.

16. A composition as claimed in any preceding claim, further comprising one or more adjunctive plasticase enzymes.

17. A composition as claimed in claim 16, wherein the adjunctive plasticase enzyme is selected from the group: (a) a laccase (EC 1.10.3.2.); (b) a manganese peroxidase (MnP, EC 1.11.1.13); (c) a lignin peroxidase (LiP, EC 1.11.1.14); and (d) combinations of two or more of the foregoing.

18. A composition as claimed in claim 17, wherein said one or more adjunctive plasticase enzymes comprise a complementary plasticase.

19. A composition as claimed in claim 18, wherein the complementary plasticase is selected from: (a) a laccase (EC 1.10.3.2.); (b) a manganese peroxidase (MnP, EC 1.11.1.13); (c) a lignin peroxidase (LiP, EC 1.11.1.14); (d) a versatile peroxidase; (e) a protease; (f) a lipase; (g) a carboxylesterases; (h) an esterase; and (i) combinations of two or more of the foregoing.

20. A composition of claim 19, wherein complementary plasticase comprises a cutinase and/or a PETase.

21. A composition of any one of the preceding claims, further comprising a waste remediation enzyme cocktail comprising one or more ancillary non-plasticase bioremediation enzymes.

22. A composition of claim 21, wherein said one or more ancillary bioremediation enzymes are selected from: (a) oxidoreductases; (b) oxygenases; (c) monooxygenases; (d) dioxygenases; (e) laccases; (f) peroxidases; (g) a hydrolase; (h) a peroxygenase, for example from Agrocybe aegerita; and (i) combinations of two or more of the foregoing.

23. A composition of claim 21 or claim 22, wherein the ancillary bioremediation enzymes comprise a hydrolase selected from: (a) a lipase; (b) a cellulase; (c) a protease; and (d) combinations of two or more of the foregoing.

24. A composition of any one of the preceding claims, further comprising one or more ancillary cellulose, hemicellulose and/or ligninase enzymes.

25. A composition of any one of the preceding claims, further comprising one or more cofactors.

26. A composition of claim 25, wherein said one or more cofactors comprises haem and/or iron and/or flavin mononucleotide (FMN) and/or FAD(H₂) and/or NAD(H).

27. A composition of any preceding claim, wherein one or more the enzymes of the composition are produced in situ, by a recombinant host expressing and secreting said enzyme.

28. A composition as claimed in claim 27, wherein two or more enzymes are produced by expression and secretion from the host.

29. A composition as claimed in claim 27 or 28, wherein the host is selected from: Escherichia coli; Bacillus subtilis; Bacillus megaterium; Bacillus licheniformis; Lactococcus lactis; Ralstonia eutropha; Pseudomonas spp; Ralstonia pickettii; Corynebacterium spp; Saccharomyces cerevisiae; Hansenula polymorpha; Komagatella phaffii; Candida biodini; Schizosaccharomyces pombe a filamentous fungus, for example Aspergillus spp. and Trichoderma spp., and Myceliophthora thermophile, optionally in the form of a C1 expression system.

30. A composition of any one of the preceding claims, further comprises one or more microorganisms selected from the following genera: Achromobacter, Acinetobacter, Actinomadura, Alcaligines, Alcanivorax, Amycolatopsis, Aneurinibacillus, Archaeoglobus, Aromatoleum, Arthrobacter, Aspergillus, Azoarcus, Actinomadura, Bionectria, Isaria, Bacillus, Pseudomonas, Fusarium, Beauveria, Brevibacillus, Brevibacterium, Brevundimonas, Candida, Chaetomium, Citrobacter, Cladosporium, Comamonas, Coriolus, Coryneformes, Corynebacterium, Cunninghamella, Curvularia, Cycloclasticus, Delftia, Desulfococcus, Desulfosarcina, Dictyoglomus, Diplococcus, Engyodontium, Enterobacter, Exiguobacterium, Flavobacterium, Gliocladium, Gordonia, Halomonas, Hansenula, Kibdelosporangium, Klebsiella, Kluyveromyces, Leptothrix, Listeria, Marinobacter, Microbacterium, Micrococcus, Moraxella, Mortierella, Mucor, Mycobacterium, Nocardia, Ochrobactrum, Oleispira, Paecylomyces, Paenibacillus, Penicillium, Phanerochaete, Pleurotus, Proteobacterium, Proteus, Pseudomonas, Pseudozyma, Pullularia, Rahnella, Ralstonia, Rhizopus, Rhodococcus, Sphingomonas, Saccharomyces, Serratia, Sphingobacterium, Sphingomonas, Streptomyces, Staphylococcus, Stenotrophomonas, Streptococcus, Talaromyces, Thalassolituus, Thermomonospora, Trametes, Trichoderma, Tritirachium, Vibrio, Xanthomonas and combinations of two or more of the foregoing.

31. A composition as claimed in any preceding claim, wherein one or more of the enzymes are immobilised on a support.

32. A composition as claimed in any preceding claim, wherein the KatG enzyme is B2UBU5 or M1FBG9.

33. A composition as claimed in any preceding claim in the form of an aqueous solution, emulsion, suspension or dispersion.

34. A composition as claimed in any preceding claim for use in the degradation of a PA polymer.

35. A composition as claimed in claim any preceding claim for use in the bioremediation of material contaminated with a PA polymer.

36. A method for the enzymatic degradation of a PA polymer, the method comprising contacting the PA polymer with a composition of any one of claims 1 to 33.

37. A method as claimed in claim 35, wherein the enzymatic degradation of the PA polymer occurs in a reactor.

38. A method for producing fragments, oligomers, isolated monomers or repeating units, monomer or repeating unit fragments and/or monomer or repeating unit derivatives of a PA polymer, the method comprising the step of degrading said polymer by contacting it with a composition according to any of claims 1 to 33.

39. A method for producing a microbial biomass comprising the steps of: (a) providing a seed culture comprising at least one microorganism expressing a KatG/EC 1.11.1.21 PAase; (b) providing a feedstock comprising a PA polymer; (c) mixing said seed culture with said feedstock to form a seeded feedstock; and (d) incubating the seeded feedstock under conditions whereby C—C bonds in the PA polymer are cleaved to produce degradation products of said polymer which are metabolized by said microorganism to yield said microbial biomass.

40. A method for producing a bioproduct comprising the steps of: (a) providing a seed culture comprising at least one microorganism expressing a KatG/EC 1.11.1.21 PAase; (b) providing a feedstock comprising a PA polymer; (c) mixing said seed culture with said feedstock to form a seeded feedstock; and (d) incubating the seeded feedstock under conditions whereby C—C bonds in the PA polymer are cleaved by said enzyme to produce degradation products of said polymer which are metabolized by said microorganism to yield said bioproduct.

41. A method of claim 40, wherein the bioproduct comprises a biofuel.

42. A method of claim 40 or 41, wherein the bioproduct comprises one or more carboxylic acids, aldehydes, or dicarboxylic acids.

43. A method of claims 40 to 42, wherein the bioproduct comprises one or more compounds selected from the group: oxalic (ethanedioic), malonic (propanedioic), succinic (butanedioic), glutaric (pentanedioic), adipic(hexanedioic), pimelic (heptanedioic), suberic (octanedioic), azelaic (nonanedioic), sebacic (decanedioic), undecanedioc and dodecanedioic acids, glycolic acid, glyoxylic acid, citric acid, furoic acid and phenylpyruvic acids.

44. A method as claimed in claims 40 to 43, wherein the PA polymer is pre-treated abiotically.

45. A method as claimed in claim 44, wherein the abiotic pre-treatment is selected from mechanical, physical and chemical treatments or a combination of two or more thereof.

46. Use of a composition as claimed in any of claims 1 to 33, for the degradation of a PA polymer.

47. Use of a composition as claimed in any of claims 1 to 33, for producing fragments, oligomers, isolated monomers or repeating units, monomer or repeating unit fragments and/or monomer or repeating unit derivatives of a PA polymer.

48. Use of a composition as claimed in any of claims 1 to 33, for the bioremediation of material contaminated with a PA polymer.

49. A KatG prodegradant comprising a KatG/EC 1.11.1.21 polyalkenase enzyme (and/or a microorganism expressing and/or secreting a KatG/EC 1.11.1.21 polyalkenase enzyme), for use as a biotic prodegradant plastic additive, optionally wherein the PAase is present in the form of an inactive proenzyme.

50. A composite KatG plastic comprising a plastic polymer in combination with the KatG prodegradant of claim 50.

51. An autodegradative PA polymer comprising a PA polymer in combination with the KatG prodegradant of claim 50.

52. A composite KatG plastic or autodegradative polymer of claim 51 or 52, wherein the KatG prodegradant is contained within or disposed on the surface of the polymer.

53. A plastic article comprising the KatG prodegradant, composite KatG plastic or autodegradative PA polymer of any one of claims 51 to 53.

54. A KatG EC 1.11.1.21 enzyme comprising one or more of the following amino acid substitutions: M2481, M248V, Y221F, Y221A, Y221L, W98A, W98F and/or R410N.

55. A composition as claimed herein wherein the KatG/EC1.11.1.21 enzyme is the enzyme of claim 53.
